(19) United States
(12) Patent Application Publication
(10) Pub. No.: US 2020/0214980 A1
(43) Pub. Date: Jul. 9, 2020

(54) NANOPARTICLE ARSENIC-PLATINUM COMPOSITIONS

(71) Applicant: Northwestern University, Evanston, IL (US)

(72) Inventors: Thomas V. O'Halloran, Chicago, IL (US); Haimei Chen, Evanston, IL (US); Andrew Mazar, Highland Park, IL (US)

(21) Appl. No.: 16/592,263

(22) Filed: Oct. 3, 2019

Related U.S. Application Data
(63) Continuation of application No. 14/246,678, filed on Apr. 7, 2014, now abandoned.

(51) Int. Cl. A61K 9/127; A61K 47/69; A61K 38/40; A61K 33/36; A61K 45/06; A61K 33/24; C07K 16/28; A61K 38/19; C07F 15/00; A61K 31/282; C07F 9/72

(52) U.S. Cl. CPC A61K 9/127; A61K 47/6913; A61K 38/40; A61K 33/36; A61K 45/06; A61K 33/24; C07K 2317/24; C07K 16/2887; A61K 38/193; C07F 15/0093; A61K 31/282; C07F 9/72; A61K 9/1271

(57) ABSTRACT

The present invention relates to nanoparticle encapsulated arsenic and platinum compositions and methods of use thereof. In particular, the present invention provides co-encapsulation of active forms of arsenic and platinum drugs into liposomes, and methods of using such compositions for the diagnosis and treatment of cancer.

The present application is a continuation of U.S. patent application Ser. No. 14/246,678, filed Apr. 7, 2014, which is a continuation of U.S. patent application Ser. No. 12/877,414, filed Sep. 8, 2010, now abandoned, which is a continuation-in-part of U.S. Pat. No. 8,246,983, issued Aug. 21, 2012,which claims priority to U.S. Provisional Application 60/713,672, filed Sep. 2, 2005; which claims priority to U.S. Provisional Application 61/240,925, filed Sep. 9, 2009, each of which are herein incorporated by reference in their entireties.

### STATEMENT REGARDING FEDERALLY SPONSORED RESEARCH OR DEVELOPMENT

This invention was made with government support under Grant Nos. U54 CA119341, GM054111, and RO1 GM38784 awarded by the National Institutes of Health. The government has certain rights in the invention.

### FIELD OF THE INVENTION

The present invention relates to nanoparticle encapsulated arsenic and platinum compositions and methods of use thereof In particular, the present invention provides co-encapsulation of active forms of arsenic and platinum drugs into liposomes, and methods of using such compositions for the diagnosis and treatment of cancer.

### BACKGROUND

Arsenic- and platinum-based drugs are highly potent but also toxic agents used in cancer therapy (Dilda & Hogg, Cancer Treat. Rev. 2007, 33, 542-564., Kelland, Nat. Rev. Cancer 2007, 7, 573-584., herein incorporated by reference in their entireties). Arsenic trioxide (TRISENOX, As₂O₃) is a front-line drug for treatment of acute promyelocytic leukemia (Wang & Chen, Blood 2008, 111, 2505-2515., herein incorporated by reference in its entirety), and is in clinical trials for treatment of other malignancies, including multiple myeloma (Berenson & Yeh, Clin. Lymphoma Myeloma 2006, 7, 192-198., herein incorporated in its entirety). However, clinical outcomes of As₂O₃ in solid tumors have been poor in many cases (Dilda & Hogg, Cancer Treat. Rev. 2007, 33, 542-564., Chen et al. Semin. Hematol. 2002, 39, 22-26., herein incorporated by reference in their entireties), mainly due to limited bioavailability of the drug in the tumor site. Clinical application to solid tumors is also impeded by toxicity including neutropenia, liver failure and cardiac toxicity (Dilda & Hogg, Cancer Treat. Rev. 2007, 33, 542-564., Evens et al. Leuk. Res. 2004, 28, 891-900., herein incorporated by reference in their entireties) at higher doses (Liu et al. Cancer Sci. 2006, 97, 675-681., herein incorporated by reference in its entirety). Cisplatin (cis-diamine dichloroplatinum(II), SEE FIG. 1) is commonly used in the treatment of a variety of solid tumors, including lung, ovarian, bladder, and testicular cancer (Kelland, Nat. Rev. Cancer 2007, 7, 573-584., herein incorporated by reference in its entirety). The active intracellular species appear to be the hydrolyzed monoaqua- and diaqua-cisplatin (aqua-cisPt, FIG. 1) (Wang & Lippard, Nat. Rev. Drug Discov. 2005, 4, 307-320., herein incorporated by reference in its entirety). Broader therapeutic applications of cisPt are limited by serious systemic toxicities, development of drug resistance, and rapid inactivation of the drug due to complexation with plasma and tissue proteins (Wang & Lippard, Nat. Rev. Drug Discov. 2005, 4, 307-320., Kelland, Nat. Rev. Cancer 2007, 7, 573-584., herein incorporated by reference in its entireties). These problems can be reduced by using a drug delivery system that prevents drug deactivation, extends the circulation time of drug in blood and increases its accumulation at tumor sites (Allen & Cullis, Science 2004, 303, 1818-1822., herein incorporated by reference in its entirety).

Lipid-based carriers have been successfully applied in clinics for improving the therapeutic efficacy of numerous drugs, such as liposomal doxorubicin (DOXIL) (Gabizon, Cancer Invest. 2001, 19, 424-436., herein incorporated by reference in its entirety), mainly via the enhanced permeability and retention (EPR) effects (Allen & Cullis, Science 2004, 303, 1818-1822., herein incorporated by reference in its entirety). Several liposomal formulations of cisPt have been prepared, including STEALTH SPI-077 (Peleg-Shulman et al. Biochim. Biophys. Acta 2001, 1510, 278-291., herein incorporated by reference in its entirety), and negative-lipid coated cisPt nanocapsules (Burger Koert et al. Nat Med 2002, 8, 81-84., herein incorporated by reference in its entirety); however, their clinical applications have been hindered by low encapsulation efficiencies (0.02 Pt-to-lipid molar ratio) which limits bioavailability (Harrington et al. Ann. Oncol. 2001, 12, 493-496., Bandak et al. Anti-Cancer Drugs 1999, 10, 911-920., herein incorporated by reference in their entireties), and poor serum stability (lifetime<1 hour) (Velinova et al. Biochim. Biophys. Acta 2004, 1663, 135-142., herein incorporated by reference in its entirety). Preparations of liposomal As₂O₃ have also faced challenges because the neutral As(OH)₃ species (which is predominant at pH<9.0) (Ni Dhubhghaill & Sadler, Struct. Bonding (Berlin) 1991, 78, 129-190., herein incorporated by reference in its entirety) diffuses readily across lipid membranes (Chen et al. J. Am. Chem. Soc. 2006, 128, 13348-13349., herein incorporated by reference in its entirety), thus making stable drug-encapsulation difficult (Kallinteri et al. J. Liposome Res. 2004, 14, 27-38., herein incorporated by reference in its entirety). Recently, the latter issues were overcome via development of an efficient system for loading high densities of As₂O₃ nanoparticulates into liposomes (0.5 drug-to-lipid molar ratio) with excellent retention (shelf life>6 months) and good serum stability (Chen et al. J. Am. Chem. Soc. 2006, 128, 13348-13349., Chen et al., Mol. Cancer Ther. 2009, WO/2007/028154, herein incorporated by reference in their entireties). This system employs transmembrane gradients of transition metal ions to produce As₂O₃ nanoparticles within liposomes. The nanoparticulate forms of a drug encapsulated in liposomes (nanobin) exhibit enhanced anticancer efficacy relative to the parent drug in both breast cancer and lymphoma xenograft, as well as reduced systemic toxicity.

### SUMMARY OF THE INVENTION

In some embodiments, the present invention provides compositions comprising liposomal nanoparticles, wherein the liposomal nanoparticle encapsulates therapeutically effective amounts of a platinum-containing compound and an arsenic-containing compound. In some embodiments, the arsenic-containing compound is selected from arsenic trioxide, arsenite, arsenious acid, arsonous acid, arsine, thioarsenious acid, arsenate, arsenic acid, arsenic acid, arsenic acid, methylarsinic acid, and dimthylarsinic acid. In some embodiments, the platinum-containing compound is selected from Cisplatin (cisPt), Monoaqua-cisPt, Aqua-cisPt, Carboplatin, Oxaliplatin, and platinum coordinating compounds. In some embodiments, the liposomal nanoparticle is stable under physiological conditions. In some embodiments, the liposome further comprises a targeting moiety. In some embodiments, the targeting moiety comprises a targeting ligand. In some embodiments, the targeting ligand is selected from folic acid, retinoic acid, a peptide, an estrogen analog, transferrin, and granulocyte-macrophage colony stimulating factor. In some embodiments, the targeting moiety comprises an antibody. In some embodiments, the antibody is selected from RITUXAN, HERCEPTIN, CAMPATH-1H, HM1.24, anti-HER2, Anti-CD38, HuM195, HP67.6, TRAIL mAb, transferin, ATN-291, and prolactin. In some embodiments, the present invention provides a method for treating cancer comprising administering the liposomal nanoparticles encapsulating therapeutically effective amounts of a platinum-containing compound and an arsenic-containing compound described herein to a subject suffering from cancer.

In some embodiments, the present invention provides a composition comprising particles having the molecular formula:

[(X₁X₂X₃X₄)Pt]ₙ[(Y₁Y₂Y₃Y₄)As]ₘ

or

[(X₁X₂X₃X₄)Pt]ₙ[(Y₁Y₂Y₃)As]ₘ

wherein X=O, OH, OH₂, N, NH₂, NH₃, S, SH, Cl, Br, F, P, Se, SeH, an amino carrier ligand, a leaving group, or an R group; wherein Y=O, OH, OH₂, N, NH₂, NH₃, S, SH, Cl, Br, F, P, Se, SeH, As, an amino carrier ligand, a leaving group, or an R group; wherein R comprises an alkyl group or an alkylidene group; wherein n is 10 or less; wherein m is 10 or less; wherein X is optionally bound to additional substituents; and wherein Y is optionally bound to additional substituents. In some embodiments, each X group comprises different substituents (e.g. OH, N, O, and P; or R, N, and OH, etc.). In some embodiments, two X groups comprise the same substituents (e.g. N, N, OH, and P; or O, O, and OH; etc.). In some embodiments, three X groups comprise the same substituents (e.g. NH₃, NH₃, NH₃, and O; or O, O, and O; etc.). In some embodiments, each X group comprise the same substituents (e.g. OH, OH, OH, and OH; etc.). In some embodiments, each Y group comprises different substituents (e.g. OH, N, O, and P; or R, N, and OH, etc.). In some embodiments, two Y groups comprise the same substituents (e.g. N, N, OH, and P; or O, O, and OH; etc.). In some embodiments, three Y groups comprise the same substituents (e.g. NH₃, NH₃, NH₃, and O; or O, O, and O; etc.). In some embodiments, each Y group comprise the same substituents (e.g. OH, OH, OH, and OH; etc.). In some embodiments, the particles comprise liposome-encapsulated nanoparticles. In some embodiments, the liposome-encapsulated nanoparticles are stable under physiological conditions. In some embodiments, the liposome-encapsulated nanoparticles further comprise a targeting moiety. In some embodiments, the targeting moiety comprises a targeting ligand. In some embodiments, the targeting ligand is selected from folic acid, retinoic acid, a peptide, an estrogen analog, transferrin, and granulocyte-macrophage colony stimulating factor. In some embodiments, the targeting moiety comprises an antibody. In some embodiments, the antibody is selected from RITUXAN, HERCEPTIN, CAMPATH-1H, HM1.24, anti-HER2, Anti-CD38, HuM195, HP67.6, TRAIL mAb, transferin, ATN-291, and prolactin. In some embodiments, the present invention provides a method for treating cancer comprising administering the liposome-encapsulated nanoparticles described herein to a subject suffering from cancer.

In some embodiments, the present invention provides a method for making a pharmaceutical preparation comprising: (a) providing: (i) a lipid composition, (ii) a platinum-containing compound, and (iii) an arsenic-containing compound, (b) combining the lipid composition and the platinum-containing compound under conditions such that the lipid compositions forms liposomes which encapsulate the platinum-containing compound, and (c) combining the liposomes and the arsenic-containing compound under conditions such that the arsenic-containing compound is encapsulated within the liposomes, wherein the platinum-containing compound is retained within the liposomes upon encapsulation of the arsenic-containing compound. In some embodiments, the method further comprises the step between steps (b) and (c) of purifying the liposomes away from unencapsulated platinum-containing compound. In some embodiments, the method further comprises the step following step (c) of purifying the liposomes away from unencapsulated arsenic-containing compound. In some embodiments, the liposomes are stable under physiological conditions. In some embodiments, the arsenic-containing compound is selected from arsenic trioxide, arsenite, arsenious acid, arsonous acid, arsine, thioarsenious acid, arsenate, arsenic acid, arsenic acid, arsenic acid, methylarsinic acid, and dimthylarsinic acid. In some embodiments, the platinum-containing compound is selected from Cisplatin (cisPt), Monoaqua-cisPt, Aqua-cisPt, Carboplatin, Oxaliplatin, and platinum coordinating compounds. In some embodiments, the present invention comprises a pharmaceutical composition produced by the methods herein. In some embodiments, the present invention provides a method of treating cancer comprising administering a pharmaceutical composition produced by the methods herein to a subject suffering from cancer.

In some embodiments, the present invention provides a method for making a pharmaceutical preparation comprising: (a) providing: (i) a lipid composition, (ii) a platinum-containing compound, and (iii) an arsenic-containing compound, (b) combining the lipid composition and the platinum-containing compound under conditions such that the lipid compositions forms liposomes which encapsulate the platinum-containing compound, and (c) combining the liposomes and the arsenic-containing compound under conditions such that the arsenic-containing compound is co-encapsulated with the platinum-containing compound within the liposomes. In some embodiments, the method further comprises the step between steps (b) and (c) of purifying the liposomes away from unencapsulated platinum-containing compound. In some embodiments, the method further comprises the step following step (c) of purifying the liposomes away from unencapsulated arsenic-containing compound. In some embodiments, the liposomes are stable under physiological conditions. In some embodiments, the arsenic-containing compound is selected from arsenic trioxide, arsenite, arsenious acid, arsonous acid, arsine, thioarsenious acid, arsenate, arsenic acid, arsenic acid, arsenic acid, methylarsinic acid, and dimthylarsinic acid. In some embodiments, the platinum-containing compound is selected from Cisplatin (cisPt), Monoaqua-cisPt, Aqua-cisPt, Carboplatin, Oxaliplatin, and platinum coordinating compounds. In some embodiments, the present invention comprises a pharmaceutical composition produced by the methods herein. In some embodiments, the present invention provides a method of treating cancer comprising administering a pharmaceutical composition produced by the methods herein to a subject suffering from cancer.

In some embodiments, the present invention provides a composition comprising a liposome, wherein the liposome encapsulates a metal and an amphiphilic drug. In some embodiments, the amphiphilic drug is an arsenic-containing drug (e.g., arsenite, arsenic trioxide, arsenic sulfide, arsenate, methylarsinic acid or dimthylarsinic acid). In some embodiments, the metal is Ni, Co, Cu, Zn, Mn, Fe, Pb, V, Ti, Cr, Pt, Rh, Ru, Mo, Hg, Ag, Gd, Cd or Pd. In preferred embodiments, the liposome is stable under physiological conditions but releases the drug at low pH. In certain embodiments, the liposome further comprises a second amphiphilic drug. In some embodiments, the liposome comprises a composition having the formula Mn(AsX3)m , wherein X is O, OH, S, SH, Se, or SeH, M is a metal ion, n is 1, 2, or 3 and m is 1, 2, or 3. In some embodiments, the liposome further comprises a targeting ligand. In preferred embodiments, the targeting ligand is an antibody (e.g., Rituxan, Campath-1H, HM1.24, HER2, Anti-CD38, HuM195, or HP67.6), folic acid, retinoic acid, a peptide, an estrogen analog, transferrin, or granulocyte-macrophage colony stimulating factor. A variety of other targeting ligands that find use with the present invention are known in the art.

The present invention further provides a method, comprising, providing a liposome; combining the liposome with a metal ion under conditions such that the metal ion is encapsulated in the liposome; and contacting the liposome comprising the encapsulated metal ion with an amphiphilic drug under conditions such that the drug is encapsulated in the liposome. In some embodiments, the amphiphilic drug is an arsenic-containing drug (e.g., arsenite, arsenic trioxide, arsenic sulfide, arsenate, methylarsinic acid or dimthylarsinic acid). In some embodiments, the metal is Ni, Co, Cu, Zn, Mn, Fe, Pb, V, Ti, Cr, Pt, Rh, Ru, Mo, Hg, Ag, Gd, Cd or Pd. In preferred embodiments, the liposome is stable under physiological conditions but releases the drug at low pH, temperature change or contact with a second liposome comprising a fluid liposome with a lower gel to crystal transition temperature than the liposome. In certain embodiments, the liposome further comprises a second amphiphilic drug. In some embodiments, the liposome comprises a composition having the formula Mn(AsX3)m , wherein X is O, OH, S, SH, Se, or SeH, M is a metal ion, n is 1, 2, or 3 and m is 1, 2, or 3. In some embodiments, the liposome further comprises a targeting ligand. In preferred embodiments, the targeting ligand is an antibody (e.g., Rituxan, Campath-1H, HM1.24, HER2, Anti-CD38, HuM195, or HP67.6), folic acid, retinoic acid, a peptide, an estrogen analog, transferrin, or granulocyte-macrophage colony stimulating factor.

The present invention additionally provides a method of treating or analyzing a cancer, comprising administering the liposomal composition comprising an amphiphilic drug described herein to a subject diagnosed with or suspected of having cancer (e.g., Lymphoma, Multiple Myeloma (MM), Acute Promyelocytic Leukemia (APL), Acute Myeloid Leukemia (AML), Chronic Lymphocytic Leukemia (CLL), breast cancer, ovarian cancer, pancreatic cancer, bladder cancer, lung cancer, liver cancer, brain cancer, neck cancer, colorectal cancer, etc.). In some embodiments, the cancer is analyzed following treatment to determine the effect of the compositions on the cancer.

Thus, the present invention describes a novel and widely applicable method of efficient and rapid loading of arsenic drugs at high density into liposomes. The method yields robust As-loaded liposomes or other lipid complexes that can retain the drug under physiological conditions. These arsenic loaded liposomes are stable in serum conditions but release their drug contents in lower pH environments, such as in the intracellular endosomes. The loading mechanism can be described as a nano-pump. For example, during one cycle, the external neutral arsenic compound, for example, As(OH)3, diffuses across the membrane to form insoluble metal arsenite complexes internally. Protons are released and associate with the basic acetate anions. The resulting weak acid (HAc) then diffuses out of the liposome in exchange for As(OH)3, leading to significant accumulation of arsenic inside liposomes. Both the formation of insoluble metal arsenite complexes and the efflux of acetic acid drive arsenic uptake.

The present invention also provides a novel way to transport the arsenic reactants into the liposome. This produces various salts of arsenous acids in nanoparticle-form. These are sequestered in a biocompatible vehicle to be delivered to cancer targets or other targets. The encapsulation methods of the present invention are applicable for other amphiphatic agents. Preferably, a therapeutic agent is one that is able to diffuse across lipid- or polymer- membranes at a reasonable rate and which is capable of coordinating with a metal encapsulated within the liposome in a prior step. Agents that are capable of coordination with a transition metal typically comprise of coordination sites such as hydroxyl, thiols, acetylenes, amines or other suitable groups capable of donating electrons to the transition metal thereby forming a complex with the metal.

The drug loading method is applicable for multi-drug co-encapsulation into one vesicle, provided that one or more therapeutic agents are first passively encapsulated inside liposomes and the second therapeutic agent is added to the external solution of said liposomes and is thus actively loaded. Two or more drugs, such as inorganic drugs of arsenic, cisplatin (cis-diaminedichloroplatinum) and its hydrolyzed products, and tetrathiomolybate and its hydrolyzed products, and organic drugs of retinoic acid and nucleoside analogues, 8-chloro- or 8-NH2-adenosine, et al. can be incorporated into liposomes by combining passive and active methods of loading.

### BRIEF DESCRIPTIPN OF DRAWINGS

The description provided herein is better understood when read in conjunction with the accompanying drawings which are included by way of example and not by way of limitation.

FIG. 1 shows molecular structures of exemplary arsenic- and platinum-based compounds.

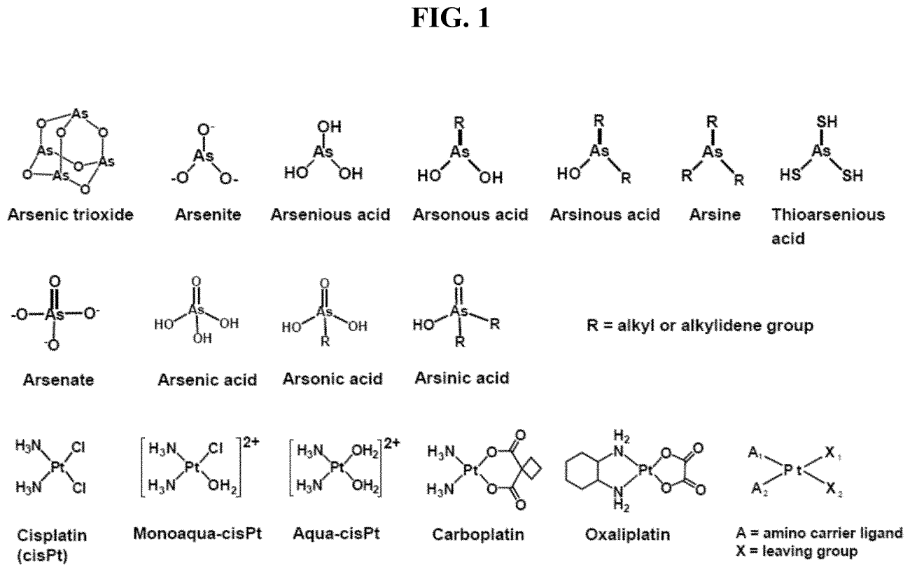

FIG. 2 shows an exemplary procedure of coencapsulating arsenic- and platinum-based drugs into liposomes. (a) Hydration of dried lipids in 300 mM aqua-cisPt acetate (pH 5.1). Self-assembled liposomes are then reduced to 100 nm by extrusion. (b) Gel exclusion for exchanging external buffer into 300 mM sucrose, 10 mM MES, pH 5.1. (c) Liposome suspension is added with As₂O₃ solution and kept at 50° C. and pH 6.6-6.9 for 11 h. (d) The influx of As(OH)₃ into liposomes to form complex(As, Pt), accompanied by the efflux of acetic acids (HAc). Removal of excess of external As(OH)₃ by gel exclusion with 300 mM sucrose, 10 mM MES, pH 7.4-8.0, followed by adjusting the pH of final liposome product back to 6.1-6.4.

FIG. 3 shows (a) the kinetics of arsenic loading into liposomes using 300 mM aqua-cisPt acetate solution (pH 5.1) as intraliposomal medium. (b) Arsenic-loading extent is dependent on the concentration of intraliposomal aqua-cisPt acetate. DPPC/DOPG/Chol=51.4/3.6/45 mol %; outer buffer: 120 mM or 300 mM sucrose, 10 mM MES, pH 6.7, 50° C. for 11 h, with an initial As-to-lipid molar ratio of 4.0.

FIG. 4 shows TEM images and EDX spectra of NB(Pt) (a, b and c) and NB(As, Pt) (d, e and f). Samples of a and d were stained by 2% uranyl acetate; b and e are unstained, showing discrete electron-dense inorganic cores within liposomes; the single-particle EDX spectra c and f correspond to b and e, respectively, revealing Pt (c), Pt and As (f) cores. Cu peaks arise from the EM grid. (g) Phase-corrected EXAFS Fourier transforms for NB(As, Pt). DPPC/DOPG/Chol=51.4/3.6/45 mol %.

FIG. 5 shows (a) XPS wide scan of complex(As, Pt)_{1.36} separated from NB(As, Pt). The C1s peak is from the carbon tape. XPS narrow region scans of Pt(4f) and As(3d) for complex(As, Pt)_{1.36} from NB(As, Pt) relative to those of aqua-cisPt acetate (b) and As₂O₃ (c).

FIG. 6 shows drug release of NB(As, Pt) as the function of time at 4° C., pH 6.1-6.4 (a) and at 37 ° C., pH 7.4 (b), and after 72 hat various pHs (c). DPPC/DOPG/Chol=51.5/3.6/45 mol %.

FIG. 7 shows a comparison of drug release of NB(As, Pt) at 37° C. in 80% FBS with various lipid compositions: DPPC/DOPG/Chol=51.4/3.6/45 (a), 86.4/3.6/10 (b), 96.4/3.6/0 (c), mol %. The faster drug release results in the higher cytotoxic effects (IC₅₀) on SU-DHL-4 (d, 72 h) and MDA-MB-231 cells (e, 96 h).

FIG. 8 shows (a) cytotoxic effects of NB(As, Pt), NB(Pt), aqua-cisPt and As₂O₃ on SU-DHL-4 cells after a 48 h incubation. For NB(As, Pt), NB(Pt), and aqua-cisPt, M=Pt; for As₂O₃, M=As. (b) Cytotoxicities (IC₅₀) of NB(As, Pt), As₂O₃, cisPt, and aqua-cisPt after incubation for 1.5 h, 24 h, 48 h, and 72 h. DPPC/DOPG/Chol=51.4/3.6/45 mol %.

FIG. 9 shows (a) cytotoxic effects of NB(As, Pt), NB(Pt), aqua-cisPt, and As₂O₃ on MDA-MB-231 cells after a 72 h incubation. For NB(As, Pt), NB(Pt) and aqua-cisPt, M=Pt; for As₂O₃, M=As. (b) Cytotoxicities (IC₅₀) of NB(As, Pt), As₂O₃, cisPt, and aqua-cisPt after incubation for 2 h, 48 h, 72 h, and 96 h. DPPC/DOPG/Chol=51.4/3.6/45 mol %.

FIG. 10 shows cytotoxic effects of NB(As, Pt), NB(Pt), cisPt and As₂O₃ on MM.1S (a) and IM-9 (b) cells after a 72 h incubation. For NB(As, Pt), NB(Pt), and cisPt, M=Pt; for As₂O₃, M=As. DPPC/DOPG/Chol=51.4/3.6/45 mol %.

FIG. 11 show preparation of folate-targeted arsenic and platinum liposomes by post-insertion of targeting ligands (Folate-PEG₃₃₅₀-DSPE) into NB(As, Pt). DPPC/DOPG/Chol=51.4/3.6/45 mol %

FIG. 12 shows comparison of cellular drug uptake and cytotoxicity of various drug formulations. Confocal micrograghs (merged with DIC images) showing cellular uptake of (a) f-NB(As, Pt), (b) f-NB(As, Pt)+2 mM FA, (c) NB(As, Pt) by KB cells, and of (d) f-NB(As, Pt) by MCF-7 cells after 3 h at 37° C. Liposomes were labeled with rhodamine (Rh). Scale bar: 20 μm. (e) KB and MCF-7 cellular arsenic and platinum uptake. Cytotoxic effects of f-NB(As, Pt) (upward pointing triangles), f-NB(As, Pt)+2 mM FA (downward-pointing triagles), NB(As, Pt) (circles), As₂O₃ (spares) and aqua-cisPt (diamonds) towards KB (f) and MCF-7 (g) cells. Cells exposed to drugs at 37° C. for 3 h, washed by PBS and further incubated up to 72 h in drug-free medium.

FIG. 13 shows structures of arsenic, platinum and molybdenum drugs used in some embodiments of the present invention.

FIG. 14 shows a schematic representation of one exemplary method of the present invention for loading arsenic into a liposome in response to a transmembrane ion gradient.

FIG. 15 shows that arsenous acids (H₃AsO₃) pass across liposome bilayers rapidly.

FIG. 16 shows arsenic loading efficiency dependent on the nature of anions of intraliposomal medium. The kinetics of arsenic loading into liposomes (DPPC/DOPG/Chol=65/5/30, wt %) with time using 300 mM Ni(O₂CCH₃)₂ (●), Ni(NO₃)₂ (▴), NiCl₂ (Δ), NiSO₄ (▪) or 142 mM Ni(O₂CH)₂ (⊙) as intraliposomal medium at pH 6.8. Outer-buffer: 300 mM (●, ▴, Δ, ▪) or 150 mM (⊙) NaCl, 20 mM HEPES and pH 7.2.

FIG. 17 shows the procedure of loading arsenic into liposomes by creating an inside to outside Ni(II) acetate (Ni(OAc)₂) gradient. (a) Dried lipids are hydrated in 300 mM Ni(OAc)₂ aqueous solution for 1.5 h at 50° C. to form 300 mM Ni(OAc)₂ encapsulated liposomes, which are thus downsized to 100 nm. (b) The external buffer of Ni(OAc)₂ is exchanged into 300 mM NaCl, 20 mM HEPES, pH 6.8 by using gel exclusion. (c) As₂O₃ or NaAsO₂ is added into liposomes at a certain As-to-Lipid molar ratio. (d) The influx of H₃AsO₃ into liposomes to form aggregation of Niₓ(AsO₃)_{y}, accompanied by the efflux of acetate acids (HAc) away from liposomes. (e) The excess of external H₃AsO₃ is removed by gel exclusion with the buffer of 300 mM NaCl, 20 mM HEPES and pH 4.0, followed by adjusting the pH of final liposome product back to 7.2.

FIG. 18 shows a comparison of intraliposomal concentrations of As³⁺and M²⁺(Ni²⁺, Co²⁺, Cu²⁺and Zn²⁺) under similar conditions at equilibrium.

FIG. 19 shows transmission electron micrography (TEM) of liposomes (DPPC/DOPG/Chol: 65/5/30, wt %) before (A) or after (B) arsenic-loading using 300 mM Ni(OAc)₂ as intraliposomal medium.

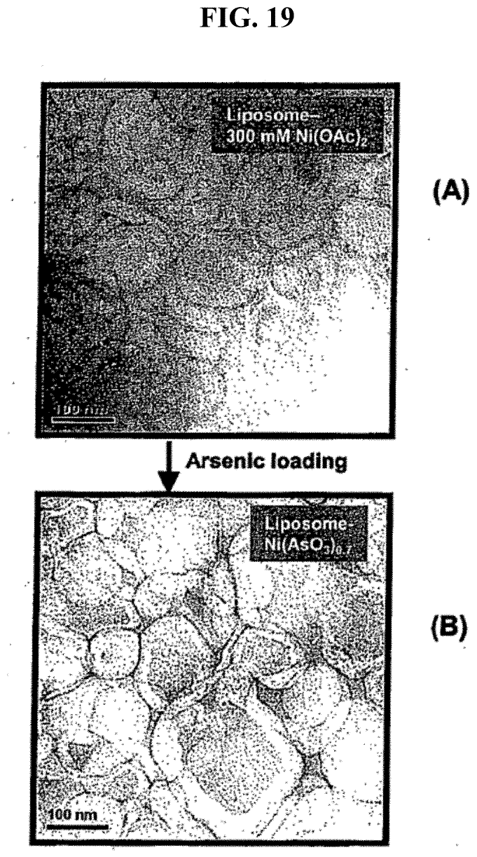

FIG. 20 shows temperature and pH triggered arsenic release from (A) Ni-arsenic-encapsulated and (B) Co-arsenic-encapsulated liposomes (DPPC/DOPG/Chol=65/5/30, wt %).

FIG. 21 shows arsenic release from Ni-arsenic-encapsulated liposomes (DOPC/DOPG/chol=65/5/30, wt %) at 4° C. (●) and 37° C. (▪).

FIG. 22 shows the extent of arsenic loading into liposomes (DPPC/DOPG/chol=65/5/30, wt %) increased with concentrations of intraliposomal Ni(O₂CCH₃)₂ or Cu(O₂CCH₃)₂ solutions.

FIG. 23 shows the kinetics of arsenic loading into liposomes (DPPC/DOPG/Chol=65/5/30, wt %) using 300 mM (A) Ni(O₂CCH₃)₂, pH 6.8, or (B) Co(O₂CCH3)₂, pH 7.2, as intraliposomal medium, with an initial As-to-Lipid molar ratio of 2.0 at 50° C.

FIG. 24 shows the cytotoxicity effect of unencapsulated ( [symbol] ) and encapsulated (Δ) and Rituxan-targeted encapsulated (⊙) arsenic drugs on SU-DHL-4 human lymphoma cells.

### DEFINITIONS

To facilitate an understanding of the present invention, a number of terms and phrases are defined below:

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “subject suspected of having cancer” refers to a subject that presents one or more symptoms indicative of a cancer or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received a preliminary diagnosis but for whom a confirmatory test has not been done or for whom the stage of cancer is not known. The term further includes people who once had cancer (e.g., an individual in remission). A “subject suspected of having cancer” is sometimes diagnosed with cancer and is sometimes found to not have cancer.

As used herein, the term “subject diagnosed with a cancer” refers to a subject who has been tested and found to have cancerous cells. The cancer may be diagnosed using any suitable method, including but not limited to, biopsy, x-ray, and blood test. A “preliminary diagnosis” is one based only on visual and antigen tests.

As used herein, the term “initial diagnosis” refers to a test result of initial cancer diagnosis that reveals the presence or absence of cancerous cells.

As used herein, the term “post surgical tumor tissue” refers to cancerous tissue that has been removed from a subject (e.g., during surgery).

As used herein, the term “subject at risk for cancer” refers to a subject with one or more risk factors for developing a specific cancer. Risk factors include, but are not limited to, gender, age, genetic predisposition, environmental expose, and previous incidents of cancer, preexisting non-cancer diseases, and lifestyle.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water and industrial samples. Such examples are not however to be construed as limiting the sample types applicable to the present invention.

As used herein, the terms “anticancer agent,” “conventional anticancer agent,” or “cancer therapeutic drug” refer to any therapeutic agents (e.g., chemotherapeutic compounds and/or molecular therapeutic compounds), radiation therapies, or surgical interventions, used in the treatment of cancer (e.g., in mammals).

As used herein, the terms “drug” and “chemotherapeutic agent” refer to pharmacologically active molecules that are used to diagnose, treat, or prevent diseases or pathological conditions in a physiological system (e.g., a subject, or in vivo, in vitro, or ex vivo cells, tissues, and organs). Drugs act by altering the physiology of a living organism, tissue, cell, or in vitro system to which the drug has been administered. It is intended that the terms “drug” and “chemotherapeutic agent” encompass anti-hyperproliferative and antineoplastic compounds as well as other biologically therapeutic compounds.

The term “derivative” of a compound, as used herein, refers to a chemically modified compound wherein the chemical modification takes place either at a functional group of the compound, aromatic ring, or carbon backbone. Such derivatives include esters of alcohol-containing compounds, esters of carboxy-containing compounds, amides of amine-containing compounds, amides of carboxy-containing compounds, imines of amino-containing compounds, acetals of aldehyde-containing compounds, ketals of carbonyl-containing compounds, and the like.

As used herein, the term “pharmaceutically acceptable salt” refers to any salt (e.g., obtained by reaction with an acid or a base) of a compound of the present invention that is physiologically tolerated in the target subject (e.g., a mammalian subject, and/or in vivo or ex vivo, cells, tissues, or organs). “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺(wherein W is a C₁₋₄ alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

A “therapeutically effective amount” is an amount sufficient to effect beneficial or desired results. An effective amount can be administered in one or more administrations.

As used herein, the term “administration” refers to the act of giving a drug, prodrug, or other agent, or therapeutic treatment (e.g., radiation therapy) to a physiological system (e.g., a subject or in vivo, in vitro, or ex vivo cells, tissues, and organs). Exemplary routes of administration to the human body can be through the eyes (opthalmic), mouth (oral), skin (transdermal), nose (nasal), lungs (inhalant), oral mucosa (buccal), ear, by injection (e.g., intravenously, subcutaneously, intratumorally, intraperitoneally, etc.) and the like.

“Coadministration” refers to administration of more than one chemical agent or therapeutic treatment to a physiological system (e.g., a subject or in vivo, in vitro, or ex vivo cells, tissues, and organs). “Coadministration” of the respective chemical agents and therapeutic treatments may be concurrent, or in any temporal order or physical combination.

The term “nanoparticle” as used herein means a particle having cross-sectional dimensions of less than about 1 micrometer. “Nanoparticles” as used herein may have cross sectional areas of less than about 750 nanometers, less than about 500 nanometers, less than about 250 nanometers, less than about 100 nanometers, or less than about 50 nanometers. As used herein, “nanoparticles” may refer to liposomal nanoparticles.

### DETAILED DESCRIPTION

In some embodiments, the present invention provides compositions and method for co-loading active forms of arsenic and platinum compounds (e.g. arsenic and platinum drugs) into liposomes. In some embodiments, the present invention provides liposomes co-loaded with active forms of arsenic and platinum compounds (e.g. arsenic and platinum drugs). In some embodiments, the present invention provides targeted delivery (e.g. within liposome, within nanoparticles, etc.) of arsenic and platinum compounds (e.g. into tumor cells). In some embodiments, the present invention provides compositions comprising liposomes and/or nanoparticles co-loaded with active arsenic and platinum compounds, methods of synthesis thereof, and methods of use thereof (e.g. methods of treating disease (e.g. cancer)) (U.S. App No. 2007/0065498, herein incorporated by reference in its entirety).

Accordingly, in some embodiments, the present invention provides compositions comprising a liposome, wherein the liposome encapsulates a platinum-containing compound (e.g. Cisplatin (cisPt), Monoaqua-cisPt, Aqua-cisPt, Carboplatin, Oxaliplatin, platinum coordinating compounds, etc.) and an arsenic-containing compound (e.g. arsenic trioxide, arsenite, arsenious acid, arsonous acid, arsine, thioarsenious acid, arsenate, arsenic acid, arsenic acid, arsenic acid, methylarsinic acid, dimthylarsinic acid, etc.). In some embodiments, the arsenic-containing compound is an arsenic-containing drug (e.g., arsenite, arsenic trioxide, arsenic sulfide, arsenate, methylarsinic acid or dimthylarsinic acid). In some embodiments, the platinum-containing compound is a platinum-containing drug.

In some embodiments, the present invention provides methods for co-loading arsenic and platinum drugs into liposomes. In some embodiments, the present invention creates transmembrane gradients of platinum containing compounds (e.g. aqua-cisplatin (aqua-cisPt)) to obtain the efficient and stable loading of a weak acid-H₃AsO₃ into liposomes by forming As^{III}-Pt^{II} nanoparticles inside. In some embodiments, anions of aqua-cisPt (e.g. acetate, formate, nitrate, lactate and hydroxyacetate) contribute to the drug loading and release processes. In some embodiments, the present invention provides forming liposomes from one or more lipid containing compositions (e.g. lipd film, phospholipids, lipid bilayer, etc.). In some embodiments, liposomes comprise one or more phospholipids (e.g. dioleoylphosphatidylethanolamine, dipalmitoylphosphatidylcholine (DPPC), dioleoylphosphatidylglycerol (DOPG), 1,2-Dipalmitoyl-sn-glycero-3-phosphoethanolamine-rhodamine (DPPE-Rh),phosphatidylcholine, sphingomyelin, lyso-phosphatidylcholine, phosphatidylglycerol, phosphatidic acid, phosphatdylethanolamine, phosphatidylserine, PEG-phospholipids, dimyristoylphosphatidylcholine, dilauroyl phosphatidylethanolamine, dihexadecylphosphatidylcholine, etc.). In some embodiments, liposomes comprise lipids (e.g. sterols, cholesterols, fatty acids, etc.). In some embodiments, liposomal nanoparticles are 10-100 μM in diameter (e.g. 10 μM, 20 μM, 30 μM, 40 μM, 50 μM, 60 μM, 70 μM, 80 μM, 90 μM, 100 μM, less than 90 μM, less than 80 μM, less than 70 μM, less than 60 μM, less than 50 μM, more than 10 μM, more than 20 μM, more than 30 μM, more than 40 μM, more than 50 μM, etc.) In some embodiments, the present invention provides forming liposomes from one or more lipid-containing compositions in the presence of one or more platinum-containing compounds. In some embodiments, liposomes are formed in the presence of one or more platinum-containing compounds under conditions (e.g. pH, temperature, pressure, catalysts, sonication, etc.) such that the resulting liposomes encapsulate the platinum-containing compounds. In some embodiments, liposomes are formed in the presence of one or more arsenic-containing compounds under conditions (e.g. pH, temperature, pressure, catalysts, sonication, etc.) such that the resulting liposomes encapsulate the arsenic-containing compounds. In some embodiments, liposomes are formed in the presence of platinum-containing and arsenic-containing compounds under conditions (e.g. pH, temperature, pressure, catalysts, sonication, etc.) such that the resulting liposomes encapsulate the platinum-containing and arsenic-containing compounds. In some embodiments, arsenic-containing compounds are added to liposomes encapsulating platinum-containing compounds, under conditions such that the liposomes encapsulate the arsenic-containing compounds while retaining the platinum-containing compounds. In some embodiments, platinum-containing compounds are added to liposomes encapsulating arsenic-containing compounds, under conditions such that the liposomes encapsulate the platinum-containing compounds while retaining the arsenic-containing compounds.

In some embodiments, liposomes, liposomal nanoparticles, and/or nanoparticles of the present invention are configured to stably encapsulate one or more arsenic-containing compounds and one or more platinum-containing compounds (e.g. stable under physiological conditions, stable under reaction conditions, stable under storage conditions, etc.). In some embodiments, liposomes, liposomal nanoparticles, and/or nanoparticles of the present invention are configured to release one or more arsenic-containing compounds and/or one or more platinum-containing compounds under release conditions (e.g. physiological conditions, specific pH range, specific temperature range, etc.). In some embodiments, liposomes, liposomal nanoparticles, and/or nanoparticles of the present invention release platinum-containing compounds and arsenic-containing compounds at a defined rate (e.g. to optimize therapeutic efficientcy).

In some embodiments, the present invention provides the synthesis of platinum- and arsenic-containing particles. In some embodiments, the present invention provides the synthesis of platinum- and arsenic-containing particles, which may be inside of liposomes, of the general formula:

[(X₁X₂X₃X₄)Pt]ₙ[(Y₁Y₂Y₃Y₄)As]ₘ

wherein X=O, OH, OH₂, N, NH2, NH3, S, SH, Cl, Br, F, P, Se, SeH, an amino carrier ligand, a leaving group, an R group, etc.;
wherein Y=O, OH, OH₂, N, NH2, NH3, S, SH, Cl, Br, F, P, Se, SeH, As, an amino carrier ligand, a leaving group, an R group, etc.;
wherein R comprises an alkyl group or an alkylidene group;
wherein n is 10 or less;
wherein m is 10 or less;
wherein X is optionally bound to additional substituents; and wherein Y is optionally bound to additional substituents.

In some embodiments, the present invention provides the synthesis of platinum- and arsenic-containing particles of the general formula:

[(X₁X₂X₃X₄)Pt]ₙ[(Y₁Y₂Y₃)As]ₘ

wherein X=O, OH, OH₂, N, NH₂, NH₃, S, SH, Cl, Br, F, P, Se, SeH, an amino carrier ligand, a leaving group, an R group, etc.;
wherein Y=O, OH, OH₂, N, NH₂, NH₃, S, SH, Cl, Br, F, P, Se, SeH, As, an amino carrier ligand, a leaving group, an R group, etc.;
wherein R comprises an alkyl group or an alkylidene group;
wherein n is 10 or less;
wherein m is 10 or less;
wherein X is optionally bound to additional substituents; and wherein Y is optionally bound to additional substituents.

In some embodiments, the present invention provides conditions (e.g. pH, buffer conditions, temperature, salt concentration, catalysts, sonication, changes thereof, etc.) which maximize liposomal-payload of platinum-containing compounds and/or arsenic-containing compounds (e.g. maximize the amount of the compounds encapsulated in the liposomes). In some embodiments, conditions (e.g. pH, buffer conditions, temperature, salt concentration, catalysts, sonication, changes thereof, etc.) maximize liposomal-retention of platinum-containing compounds and/or arsenic-containing compounds (e.g. maximize the amount of the compounds encapsulated in the liposomes). In some embodiments, the present invention provides conditions (e.g. pH, buffer conditions, temperature, salt concentration, catalysts, sonication, changes thereof, etc.) configured to provide release of platinum-containing compounds and/or arsenic-containing compounds from liposomes (e.g. controlled release). In some embodiments, nanoparticles of the present invention have low drug-release rates in serum. In some embodiments, release rates of the present invention ensure significant stability of liposomal nanoparticles upon intravenous administration. In some embodiments, the present invention provides methods of drug release triggered by temperature and/or pH (e.g. by employing liposomes with low cholesterol contents, by employing liposomes comprised of fluid lipids with lower gel-to-crystal transitional temperatures (Tₘ) and of the temperature-sensitive lipids, by employing liposomes with a pH-sensitive polymer coating, etc.).

In some embodiments, the present invention provides encapsulation methods that are applicable for additional amphipathic agents. In some, embodiments, the present invention comprises liposomes which encapsulate platinum-containing compounds, arsenic-containing compounds, and an additional agent (e.g. therapeutic agent, chemotherapeutic agent, amphipathic agents, etc.). In some embodiments, an additional agents comprises a therapeutic agent that is able to diffuse across lipid- or polymer- membranes at a reasonable rate and which is capable of coordinating with a metal (such as Pt(II)) encapsulated within the liposome. In some embodiments, the present invention provides encapsulation methods that are applicable for multi-drug co-encapsulation into one vesicle. In some embodiments, one or more first therapeutic agents are passively encapsulated inside liposomes. In some embodiments, one or more second therapeutic agents are added to the external solution of said liposomes and actively loaded. In some embodiments, two or more drugs, such as inorganic drugs of arsenic trioxide, arsenite, arsenious acid and its alkyl products (arsine), arsenic sulfide, arsenate, arsenic acid and its alkyl products, cisplatin and its hydrolyzed products, carboplatin and its hydrolyzed products, oxaliplatin and its hydrolyzed products, and organic agents of nucleoside analogues (such as 8-chloro- or 8-NH₂-adenosine), retinoic acid, ascorbic acid, L-buthionine-sulfoximine, docosahexaenoic acid, et al. can be incorporated into liposomes by combining passive and active methods of loading.

In some embodiments, nanoparticle and liposomal compositions of the present invention have a long shelf life (e.g. weeks, months, years, etc). In some embodiments, nanoparticle and liposomal compositions of the present invention meet the pharmaceutical requirements for clinical use. In some embodiments, nanoparticle and liposomal compositions of the present invention do not require “bedside” preparation.

In some embodiments, the present invention provides methods of preparation of arsenic and platinum-coencapsulated liposomes with a broad spectrum of types, sizes, and composition, including sterically-stabilized liposomes and ligand-targeted liposomes. In some embodiments, the encapsules can be any suitable type of vesicle (e.g. liposomes, polymer-caged liposomes, lipid emulsions, micelles, and nano- or micro-spheres).

In some embodiments, the present invention provides methods of coupling liposomal [(X₁X₂X₃)As]ₙ[(Y₁Y₂Y₃Y₄)Pt]ₘ nanoparticles and/or [(X₁X₂X₃X₄)As]ₙ[(Y₁Y₂Y₃Y₄)Pt]ₘ nanoparticles to targeting ligands, such as folate, and of evaluating cytotoxicity of targeted-liposomes on the human nasopharyngeal epidermal carcinoma KB cells. In some embodiments, such ligand-targeted liposomal [(X₁X₂X₃)As]ₙ[(Y₁Y₂Y₃Y₄)Pt]ₘ and/or [(X₁X₂X₃X₄)As]ₙ[(Y₁Y₂Y₃Y₄)Pt]ₘ have higher anticancer efficacy than the parent arsenic and platinum drugs. The targeting ligands applicable to this invention can be, for example, folic acid and its derivatives, retinoic acid, a peptide (such as hPL), estrogen analogs such as galactosamine, Arg-Gly-Asp tripeptide (RGD), Asn-Gly-Arg (NGR), Octreotide, Granulocyte-macrophage colony-stimulating factor (GM-CSF), and other similar or suitable ligands. In some embodiments, the present invention provides targeting antibodies and proteins, including RITUXAN, HERCEPTIN, CAMPATH-1H, HM1.24, anti-HER2, Anti-CD38, HuM195, HP67.6, TRAIL mAb, transferin, prolactin, and any other suitable antibodies (e.g. antibodies to cancer markers, antibodies to disease markers, etc.). In some embodiments, the present invention provides methods of coupling aresinc- and platinum-loaded liposomal nano-particles to antibodies, such as RITUXAN, and of evaluating cytotoxicity of conjugates on the human B-cell lymphoma SU-SHL-4. Such ligand-targeted liposomes are effective therapeutics and exhibit lower toxicity as compared with the parent drugs. The targeting antibodies applicable to this invention can be various types of antibodies, including, but not limited to, RITUXAN, CAMPATH-1H, HM1.24, anti-HER2, Anti-CD38, HuM195, HP67.6. Non-antibody ligands include, for example, including, but not limited to, folate, retinoic acid, estrogen analogs such as galactosamine, Arg-Gly-Asp tripeptide (RGD), Asn-Gly-Arg (NGR), Octreotide, Granulocyte-macrophage colony-stimulating factor (GM-CSF), and proteins, such as transferrin are also suitable for use with the present invention. The present invention further provides a method of preparing and using ligand-targeted aresinc- and platinum-loaded liposomal nano-praticles for treatment of various types of disease, cancers and tumors. In some embodiments, the present invention provides antibodies that target tumors that express one or more cancer and/or tumor markers. Any suitable antibody (e.g., monoclonal, polyclonal, or synthetic) may be utilized in the therapeutic methods disclosed herein. In preferred embodiments, the antibodies used for cancer therapy are humanized antibodies. Methods for humanizing antibodies are well known in the art (See e.g., U.S. Pat. Nos. 6,180,370, 5,585,089, 6,054,297, and 5,565,332; each of which is herein incorporated by reference).

In some embodiments, the present invention provides therapies for cancer and cancer-related illnesses (e.g. Acute Lymphoblastic Leukemia, Acute Myeloid Leukemia, Adrenocortical Carcinoma, AIDS-Related Cancers, AIDS-Related Lymphoma, Anal Cancer, Appendix Cancer, Astrocytoma, Atypical Teratoid/Rhabdoid Tumor, Basal Cell Carcinoma, Bile Duct Cancer, Bladder Cancer, bone cancer (e.g. Osteosarcoma or Malignant Fibrous Histiocytoma), Brain Stem Glioma, Brain Tumor (e.g. Adult, Childhood, Brain Stem Glioma, Atypical Teratoid/Rhabdoid Tumor, Embryonal Tumors, Cerebellar Astrocytoma, Cerebral Astrocytoma, Malignant Glioma, Craniopharyngioma, Ependymoblastoma, Ependymoma, Medulloblastoma, Medulloepithelioma, Pineal Parenchymal Tumors of Intermediate Differentiation, Supratentorial Primitive Neuroectodermal Tumors and Pineoblastoma, Visual Pathway and Hypothalamic Glioma, Brain and Spinal Cord Tumors), Breast Cancer, Bronchial Tumors, Burkitt Lymphoma, Carcinoid Tumor, Carcinoma, Atypical Teratoid/Rhabdoid Tumor, Embryonal Tumors, Central Nervous System Lymphoma, Cerebellar Astrocytoma, Cervical Cancer, Childhood Cancers, Chordoma, Chronic Lymphocytic Leukemia, Chronic Myelogenous Leukemia, Chronic Myeloproliferative Disorders, Colon Cancer, Colorectal Cancer, Craniopharyngioma, Cutaneous T-Cell Lymphoma, Embryonal Tumors, Endometrial Cancer, Ependymoblastoma, Ependymoma, Esophageal Cancer, Ewing Family of Tumors, Extracranial Germ Cell Tumor, Extragonadal Germ Cell Tumor, Extrahepatic Bile Duct Cancer, Eye Cancer (e.g. Intraocular Melanoma, Retinoblastoma, etc.), Gallbladder Cancer, Gastric (Stomach) Cancer, Gastrointestinal Carcinoid Tumor, Gastrointestinal Stromal Tumor (GIST), Germ Cell Tumor (e.g. Extracranial, Extragonadal, Ovarian, etc.), Gestational Trophoblastic Tumor, Glioma (e.g., Adult, Childhood, Brain Stem, Cerebral Astrocytoma, Visual Pathway and Hypothalamic, etc.), Hairy Cell Leukemia, Head and Neck Cancer, Hepatocellular (Liver) Cancer, Hodgkin Lymphoma, Hypopharyngeal Cancer, Hypothalamic and Visual Pathway Glioma, Intraocular Melanoma, Islet Cell Tumors (Endocrine Pancreas), Kaposi Sarcoma, Kidney (Renal Cell) Cancer, Laryngeal Cancer, Leukemia (e.g. Acute, Lymphoblastic, Adult, Childhood, Acute Myeloid, Chronic Lymphocytic, Chronic Myelogenous, Hairy Cell, etc.), Lip and Oral Cavity Cancer, Liver Cancer, Lung Cancer (e.g. Non-Small Cell, Small Cell, etc.), Lymphoma (e.g. AIDS-Related, Burkitt, Cutaneous T-Cell, Mycosis Fungoides, Sézary Syndrome, Hodgkin, Adult, Childhood, Non-Hodgkin, Primary Central Nervous System, etc.), Macroglobulinemia, Malignant Fibrous Histiocytoma of Bone and Osteosarcoma, Medulloblastoma, Medulloepithelioma, Melanoma, Merkel Cell Carcinoma, Mesothelioma, Metastatic Squamous Neck Cancer, Mouth Cancer, Multiple Endocrine Neoplasia Syndrome, Multiple Myeloma/Plasma Cell Neoplasm, Mycosis Fungoides, Myelodysplastic Syndromes, Myelodysplastic/Myeloproliferative Diseases, Myelogenous Leukemia (e.g. Chronic, Acute, etc.), Myeloid Leukemia, Myeloma, Myeloproliferative Disorders, Nasal Cavity and Paranasal Sinus Cancer, Nasopharyngeal Cancer, Neuroblastoma, Oral Cancer, Oropharyngeal Cancer, Osteosarcoma and Malignant Fibrous Histiocytoma of Bone, Ovarian Cancer (e.g. Childhood, Ovarian Epithelial Cancer, Ovarian Germ Cell Tumor, Ovarian Low Malignant Potential Tumor, etc.), Pancreatic Cancer, Islet Cell Tumors, Papillomatosis, Paranasal Sinus and Nasal Cavity Cancer, Parathyroid Cancer, Penile Cancer, Pharyngeal Cancer, Pheochromocytoma, Pineal Parenchymal Tumors of Intermediate Differentiation, Pineoblastoma and Supratentorial Primitive Neuroectodermal Tumors, Pituitary Tumor, Plasma Cell Neoplasm/Multiple Myeloma, Pleuropulmonary Blastoma, Pregnancy and Breast Cancer, Primary Central Nervous System Lymphoma, Prostate Cancer, Rectal Cancer, Renal Cell (Kidney) Cancer, Renal Pelvis and Ureter, Respiratory Tract Carcinoma Involving the NUT Gene on Chromosome 15, Retinoblastoma, Rhabdomyosarcoma, Salivary Gland Cancer, Sarcoma, (e.g. Ewing Family of Tumors, Kaposi, Soft Tissue, Adult, childhood, Uterine, etc.), Sézary Syndrome, Skin Cancer (e.g. Nonmelanoma, Childhood, Melanoma, Carcinoma, Merkel Cell, etc.)Small Intestine Cancer, Soft Tissue Sarcoma, Squamous Cell Carcinoma, Squamous Neck Cancer with Occult Primary, Stomach (Gastric) Cancer, Supratentorial Primitive Neuroectodermal Tumors, T-Cell Lymphoma, Testicular Cancer, Throat Cancer, Thymoma and Thymic Carcinoma, Thyroid Cancer, Transitional Cell Cancer of the Renal Pelvis and Ureter, Trophoblastic Tumor, Unknown Primary Site, Unusual Cancers of Childhood Ureter and Renal Pelvis, Urethral Cancer, Uterine Cancer (e.g. Endometrial, Uterine Sarcoma, etc.), Vaginal Cancer, Visual Pathway and Hypothalamic Glioma, Vulvar Cancer, Waldenström Macroglobulinemia, Wilms Tumor, etc.). In some embodiments, the present invention provides a method of preparing liposomal nanoparticles for treatment of various types of tumors, especially hematological tumors, such as Lymphoma, Multiple Myeloma (MM), Acute Promyelocytic Leukemia (APL), Acute Myeloid Leukemia (AML), Chronic Lymphocytic Leukemia (CLL), and solid tumors, such as breast, ovarian, pancreatic, bladder, lung, liver, brain, neck, colorectal and nasopharyngeal cancers.

In some embodiments, the present invention provides pharmaceutical compositions used for the treatment of cancer. In some embodiments of the present invention, pharmaceutical compositions are used for the treatment of cancer by the reduction of tumor load. Within such methods, the pharmaceutical compositions described herein are administered to a patient, typically a warm-blooded animal (e.g. a human). A patient may or may not be afflicted with cancer. Accordingly, the above pharmaceutical compositions may be used to prevent the development of a cancer, treat a patient afflicted with a cancer, or prevent a reoccurrence of cancer. Pharmaceutical compositions and vaccines may be administered either prior to or following surgical removal of primary tumors and/or treatment, such as administration of radiotherapy or conventional chemotherapeutic drugs. As discussed herein, administration of the pharmaceutical compositions may be by any suitable method, including administration by intravenous, intraperitoneal, intramuscular, subcutaneous, intranasal, intradermal, anal, vaginal, topical and oral routes.

In some embodiments, the present invention provides therapies that kill cancer cells, induce apoptosis in cancer cells, stop or slow the spread of cancer, stop or reduce cancer metastasis, stop or reduce tumor formation, reduce tumor load, minimize the effects of cancer, support the ability of the body to fight cancer, and/or serve as an antagonist to cancer, cancer cells, or cancer-related diseases. In some embodiments, the present invention provides compositions, systems, methods, regents, and/or kits that provide cancer therapies.

In some embodiments, the compositions of the present invention are provided in combination with existing therapies. In other embodiments, two or more compounds of the present invention are provided in combination. In some embodiments, the compounds of the present invention are provided in combination with known cancer chemotherapy agents. In some embodiments, known chemotherapy agents are co-encapsulated in liposomal nanoparticles of the present invention. In some embodiments, chemotherapeutics which are not co-encapsulated, are co-administered with nanoparticles of the present invention. The present invention is not limited to a particular chemotherapy agent.

Various classes of antineoplastic (e.g., anticancer) agents are contemplated for use in certain embodiments of the present invention. Anticancer agents suitable for use with the present invention include, but are not limited to, agents that induce apoptosis, agents that inhibit adenosine deaminase function, inhibit pyrimidine biosynthesis, inhibit purine ring biosynthesis, inhibit nucleotide interconversions, inhibit ribonucleotide reductase, inhibit thymidine monophosphate (TMP) synthesis, inhibit dihydrofolate reduction, inhibit DNA synthesis, form adducts with DNA, damage DNA, inhibit DNA repair, intercalate with DNA, deaminate asparagines, inhibit RNA synthesis, inhibit protein synthesis or stability, inhibit microtubule synthesis or function, and the like.

In some embodiments, exemplary anticancer agents suitable for use in compositions and methods of the present invention include, but are not limited to: 1) alkaloids, including microtubule inhibitors (e.g., vincristine, vinblastine, and vindesine, etc.), microtubule stabilizers (e.g., paclitaxel (TAXOL), and docetaxel, etc.), and chromatin function inhibitors, including topoisomerase inhibitors, such as epipodophyllotoxins (e.g., etoposide (VP-16), and teniposide (VM-26), etc.), and agents that target topoisomerase I (e.g., camptothecin and isirinotecan (CPT-11), etc.); 2) covalent DNA-binding agents (alkylating agents), including nitrogen mustards (e.g., mechlorethamine, chlorambucil, cyclophosphamide, ifosphamide, and busulfan (MYLERAN), etc.), nitrosoureas (e.g., carmustine, lomustine, and semustine, etc.), and other alkylating agents (e.g., dacarbazine, hydroxymethylmelamine, thiotepa, and mitomycin, etc.); 3) noncovalent DNA-binding agents (antitumor antibiotics), including nucleic acid inhibitors (e.g., dactinomycin (actinomycin D), etc.), anthracyclines (e.g., daunorubicin (daunomycin, and cerubidine), doxorubicin (adriamycin), and idarubicin (idamycin), etc.), anthracenediones (e.g., anthracycline analogues, such as mitoxantrone, etc.), bleomycins (BLENOXANE), etc., and plicamycin (mithramycin), etc.; 4) antimetabolites, including antifolates (e.g., methotrexate, FOLEX, and MEXATE, etc.), purine antimetabolites (e.g., 6-mercaptopurine (6-MP, PURINETHOL), 6-thioguanine (6-TG), azathioprine, acyclovir, ganciclovir, chlorodeoxyadenosine, 2-chlorodeoxyadenosine (CdA), and 2′-deoxycoformycin (pentostatin), etc.), pyrimidine antagonists (e.g., fluoropyrimidines (e.g., 5-fluorouracil (ADRUCIL), 5-fluorodeoxyuridine (FdUrd) (floxuridine)) etc.), and cytosine arabinosides (e.g., CYTOSAR (ara-C) and fludarabine, etc.); 5) enzymes, including L-asparaginase, and hydroxyurea, etc.; 6) hormones, including glucocorticoids, antiestrogens (e.g., tamoxifen, etc.), nonsteroidal antiandrogens (e.g., flutamide, etc.), and aromatase inhibitors (e.g., anastrozole (ARIMIDEX), etc.); 7) platinum compounds (e.g., cisplatin and carboplatin, etc.); 8) monoclonal antibodies conjugated with anticancer drugs, toxins, and/or radionuclides, etc.; 9) biological response modifiers (e.g., interferons (e.g., IFN-α, etc.) and interleukins (e.g., IL-2, etc.), etc.); 10) adoptive immunotherapy; 11) hematopoietic growth factors; 12) agents that induce tumor cell differentiation (e.g., all-trans-retinoic acid, etc.); 13) gene therapy techniques; 14) antisense therapy techniques; 15) tumor vaccines; 16) therapies directed against tumor metastases (e.g., batimastat, etc.); 17) angiogenesis inhibitors; 18) proteosome inhibitors (e.g., VELCADE); 19) inhibitors of acetylation and/or methylation (e.g., HDAC inhibitors); 20) modulators of NF kappa B; 21) inhibitors of cell cycle regulation (e.g., CDK inhibitors); 22) modulators of p53 protein function; and 23) radiation.

Any oncolytic agent that is routinely used in a cancer therapy context finds use in the compositions and methods of the present invention. For example, the U.S. Food and Drug Administration maintains a formulary of oncolytic agents approved for use in the United States. International counterpart agencies to the U.S.F.D.A. maintain similar formularies. Table 1 provides a list of exemplary antineoplastic agents approved for use in the U.S. Those skilled in the art will appreciate that the “product labels” required on all U.S. approved chemotherapeutics describe approved indications, dosing information, toxicity data, and the like, for the exemplary agents.

TABLE 1

| | | |
|---|---|---|
| Aldesleukin | PROLEUKIN | Chiron Corp., |
| (des-alanyl-1, serine-125 human | | Emeryville, CA |
| interleukin-2) | | |
| Alemtuzumab | CAMPATH | Millennium and |
| (IgG1κ anti CD52 antibody) | | ILEX Partners, LP, |
| | | Cambridge, MA |
| Alitretinoin | PANRETIN | Ligand |
| (9-cis-retinoic acid) | | Pharmaceuticals, Inc., |
| | | San Diego CA |
| Allopurinol | ZYLOPRIM | GlaxoSmithKline, |
| (1,5-dihydro-4 H-pyrazolo[3,4- | | Research Triangle |
| d]pyrimidin-4-one monosodium salt) | | Park, NC |
| Altretamine | HEXALEN | US Bioscience, West |
| (N,N,N′,N′,N″,N″,-hexamethyl-1,3,5- | | Conshohocken, PA |
| triazine-2,4,6-triamine) | | |
| Amifostine | ETHYOL | US Bioscience |
| (ethanethiol, 2-[(3-aminopropyl)amino]-, | | |
| dihydrogen phosphate (ester)) | | |
| Anastrozole | ARIMIDEX | AstraZeneca |
| (1,3-Benzenediacetonitrile, a,a,a′,a′- | | Pharmaceuticals, LP, |
| tetramethyl-5-(1H-1,2,4-triazol-1- | | Wilmington, DE |
| ylmethyl)) | | |
| Arsenic trioxide | TRISENOX | Cell Therapeutic, |
| | | Inc., Seattle, WA |
| Asparaginase | ELSPAR | Merck & Co., Inc., |
| (L-asparagine amidohydrolase, type EC-2) | | Whitehouse Station, |
| | | NJ |
| BCG Live | TICE BCG | Organon Teknika, |
| (lyophilized preparation of an attenuated | | Corp., Durham, NC |
| strain of Mycobacterium bovis (Bacillus | | |
| Calmette-Gukin [BCG], substrain | | |
| Montreal) | | |
| bexarotene capsules | TARGRETIN | Ligand |
| (4-[1-(5,6,7,8-tetrahydro-3,5,5,8,8- | | Pharmaceuticals |
| pentamethyl-2-napthalenyl) ethenyl] | | |
| benzoic acid) | | |
| bexarotene gel | TARGRETIN | Ligand |
| | | Pharmaceuticals |
| Bleomycin | BLENOXANE | Bristol-Myers Squibb |
| (cytotoxic glycopeptide antibiotics | | Co., NY, NY |
| produced by Streptomyces verticillus; | | |
| bleomycin A₂ and bleomycin B₂) | | |
| Capecitabine | XELODA | Roche |
| (5′-deoxy-5-fluoro-N- | | |
| [(pentyloxy)carbonyl]-cytidine) | | |
| Carboplatin | PARAPLATIN | Bristol-Myers Squibb |
| (platinum, diammine [1,1- | | |
| cyclobutanedicarboxylato(2-)-0,0′]-,(SP-4- | | |
| 2)) | | |
| Carmustine | BCNU, BICNU | Bristol-Myers Squibb |
| (1,3-bis(2-chloroethyl)-1-nitrosourea) | | |
| Carmustine with Polifeprosan 20 Implant | GLIADEL WAFER | Guilford |
| | | Pharmaceuticals, Inc., |
| | | Baltimore, MD |
| Celecoxib | CELEBREX | Searle |
| (as 4-[5-(4-methylphenyl)-3- | | Pharmaceuticals, |
| (trifluoromethyl)-1H-pyrazol-1-yl] | | England |
| benzenesulfonamide) | | |
| Chlorambucil | LEUKERAN | GlaxoSmithKline |
| (4-[bis(2chlorethyl)amino]benzenebutanoic | | |
| acid) | | |
| Cisplatin | PLATINOL | Bristol-Myers Squibb |
| (PtCl₂H₆N₂) | | |
| Cladribine | LEUSTATIN, 2-CDA | R.W. Johnson |
| (2-chloro-2′-deoxy-b-D-adenosine) | | Pharmaceutical |
| | | Research Institute, |
| | | Raritan, NJ |
| Cyclophosphamide | CYTOXAN, NEOSAR | Bristol-Myers Squibb |
| (2-[bis(2-chloroethyl)amino] tetrahydro- | | |
| 2H-13,2-oxazaphosphorine 2-oxide | | |
| monohydrate) | | |
| Cytarabine | CYTOSAR-U | Pharmacia & Upjohn |
| (1-b-D-Arabinofuranosylcytosine, | | Company |
| C₉H₁₃N₃O₅) | | |
| cytarabine liposomal | DEPOCYT | Skye |
| | | Pharmaceuticals, Inc., |
| | | San Diego, CA |
| Dacarbazine | DTIC-DOME | Bayer AG, |
| (5-(3,3-dimethyl-1-triazeno)-imidazole-4- | | Leverkusen, |
| carboxamide (DTIC)) | | Germany |
| Dactinomycin, actinomycin D | COSMEGEN | Merck |
| (actinomycin produced by Streptomyces | | |
| parvullus, C₆₂H₈₆N₁₂O₁₆) | | |
| Darbepoetin alfa | ARANESP | Amgen, Inc., |
| (recombinant peptide) | | Thousand Oaks, CA |
| daunorubicin liposomal | DANUOXOME | Nexstar |
| ((8S-cis)-8-acetyl-10-[(3-amino-2,3,6- | | Pharmaceuticals, Inc., |
| trideoxy-á-L-lyxo-hexopyranosyl)oxy]- | | Boulder, CO |
| 7,8,9,10-tetrahydro-6,8,11-trihydroxy-1- | | |
| methoxy-5,12-naphthacenedione | | |
| hydrochloride) | | |
| Daunorubicin HCl, daunomycin | CERUBIDINE | Wyeth Ayerst, |
| ((1 S,3 S)-3-Acetyl-1,2,3,4,6,11- | | Madison, NJ |
| hexahydro-3,5,12-trihydroxy-10-methoxy- | | |
| 6,11-dioxo-1-naphthacenyl 3-amino-2,3,6- | | |
| trideoxy-(alpha)-L-lyxo-hexopyranoside | | |
| hydrochloride) | | |
| Denileukin diftitox | ONTAK | Seragen, Inc., |
| (recombinant peptide) | | Hopkinton, MA |
| Dexrazoxane | ZINECARD | Pharmacia & Upjohn |
| ((S)-4,4′-(1-methyl-1,2-ethanediyl)bis-2,6- | | Company |
| piperazinedione) | | |
| Docetaxel | TAXOTERE | Aventis |
| ((2R,3S)-N-carboxy-3-phenylisoserine, N- | | Pharmaceuticals, Inc., |
| tert-butyl ester, 13-ester with 5b-20-epoxy- | | Bridgewater, NJ |
| 12a,4,7b,10b,13a-hexahydroxytax-11-en- | | |
| 9-one 4-acetate 2-benzoate, trihydrate) | | |
| Doxorubicin HCl | ADRIAMYCIN, | Pharmacia & Upjohn |
| (8S,10S)-10-[(3-amino-2,3,6-trideoxy-a-L- | RUBEX | Company |
| lyxo-hexopyranosyl)oxy]-8-glycolyl- | | |
| 7,8,9,10-tetrahydro-6,8,11-trihydroxy-1- | | |
| methoxy-5,12-naphthacenedione | | |
| hydrochloride) | | |
| doxorubicin | ADRIAMYCIN PFS | Pharmacia & Upjohn |
| | INTRAVENOUS | Company |
| | INJECTION | |
| doxorubicin liposomal | DOXIL | Sequus |
| | | Pharmaceuticals, Inc., |
| | | Menlo park, CA |
| dromostanolone propionate | DROMOSTANOLONE | Eli Lilly & Company, |
| (17b-Hydroxy-2a-methyl-5a-androstan-3- | | Indianapolis, IN |
| one propionate) | | |
| dromostanolone propionate | MASTERONE | Syntex, Corp., Palo |
| | INJECTION | Alto, CA |
| Elliott's B Solution | ELLIOTT'S B | Orphan Medical, Inc |
| | SOLUTION | |
| Epirubicin | ELLENCE | Pharmacia & Upjohn |
| ((8S-cis)-10-[(3-amino-2,3,6-trideoxy-a-L- | | Company |
| arabino-hexopyranosyl)oxy]-7,8,9,10- | | |
| tetrahydro-6,8,11-trihydroxy-8- | | |
| (hydroxyacetyl)-1-methoxy-5,12- | | |
| naphthacenedione hydrochloride) | | |
| Epoetin alfa | EPOGEN | Amgen, Inc |
| (recombinant peptide) | | |
| Estramustine | EMCYT | Pharmacia & Upjohn |
| (estra-1,3,5(10)-triene-3,17-diol(17(beta))-, | | Company |
| 3-[bis(2-chloroethyl)carbamate] 17- | | |
| (dihydrogen phosphate), disodium salt, | | |
| monohydrate, or estradiol 3-[bis(2- | | |
| chloroethyl)carbamate] 17-(dihydrogen | | |
| phosphate), disodium salt, monohydrate) | | |
| Etoposide phosphate | ETOPOPHOS | Bristol-Myers Squibb |
| (4′-Demethylepipodophyllotoxin 9-[4,6-O- | | |
| (R)-ethylidene-(beta)-D-glucopyranoside], | | |
| 4′-(dihydrogen phosphate)) | | |
| etoposide, VP-16 | VEPESID | Bristol-Myers Squibb |
| (4′-demethylepipodophyllotoxin 9-[4,6-0- | | |
| (R)-ethylidene-(beta)-D-glucopyranoside]) | | |
| Exemestane | AROMASIN | Pharmacia & Upjohn |
| (6-methylenandrosta-1,4-diene-3,17-dione) | | Company |
| Filgrastim | NEUPOGEN | Amgen, Inc |
| (r-metHuG-CSF) | | |
| floxuridine (intraarterial) | FUDR | Roche |
| (2′-deoxy-5-fluorouridine) | | |
| Fludarabine | FLUDARA | Berlex Laboratories, |
| (fluorinated nucleotide analog of the | | Inc., Cedar Knolls, |
| antiviral agent vidarabine, 9-b-D- | | NJ |
| arabinofuranosyladenine (ara-A)) | | |
| Fluorouracil, 5-FU | ADRUCIL | ICN Pharmaceuticals, |
| (5-fluoro-2,4(1H,3H)-pyrimidinedione) | | Inc., Humacao, |
| | | Puerto Rico |
| Fulvestrant | FASLODEX | IPR Pharmaceuticals, |
| (7-alpha-[9-(4,4,5,5,5-penta | | Guayama, Puerto |
| fluoropentylsulphinyl) nonyl]estra-1,3,5- | | Rico |
| (10)-triene-3,17-beta-diol) | | |
| Gemcitabine | GEMZAR | Eli Lilly |
| (2′-deoxy-2′,2′-difluorocytidine | | |
| monohydrochloride (b-isomer)) | | |
| Gemtuzumab Ozogamicin | MYLOTARG | Wyeth Ayerst |
| (anti-CD33 hP67.6) | | |
| Goserelin acetate | ZOLADEX IMPLANT | AstraZeneca |
| (acetate salt of [D- | | Pharmaceuticals |
| Ser(But)⁶,Azgly¹⁰]LHRH; pyro-Glu-His- | | |
| Trp-Ser-Tyr-D-Ser(But)-Leu-Arg-Pro- | | |
| Azgly-NH2 acetate | | |
| [C₅₉H₈₄N₁₈O₁₄•(C₂H₄O₂)ₓ | | |
| Hydroxyurea | HYDREA | Bristol-Myers Squibb |
| Ibritumomab Tiuxetan | ZEVALIN | Biogen IDEC, Inc., |
| (immunoconjugate resulting from a | | Cambridge MA |
| thiourea covalent bond between the | | |
| monoclonal antibody Ibritumomab and the | | |
| linker-chelator tiuxetan [N-[2- | | |
| bis(carboxymethyl)amino]-3-(p- | | |
| isothiocyanatophenyl)-propyl]-[N-[2- | | |
| bis(carboxymethyl)amino]-2-(methyl)- | | |
| ethyl]glycine) | | |
| Idarubicin | IDAMYCIN | Pharmacia & Upjohn |
| (5,12-Naphthacenedione, 9-acetyl-7-[(3- | | Company |
| amino-2,3,6-trideoxy-(alpha)-L-lyxo- | | |
| hexopyranosyl)oxy]-7,8,9,10-tetrahydro- | | |
| 6,9,11-trihydroxyhydrochloride, (7S-cis)) | | |
| Ifosfamide | IFEX | Bristol-Myers Squibb |
| (3-(2-chloroethyl)-2-[(2- | | |
| chloroethyl)amino]tetrahydro-2H-1,3,2- | | |
| oxazaphosphorine 2-oxide) | | |
| Imatinib Mesilate | GLEEVEC | Novartis AG, Basel, |
| (4-[(4-Methyl-1-piperazinyl)methyl]-N-[4- | | Switzerland |
| methyl-3-[[4-(3-pyridinyl)-2- | | |
| pyrimidinyl]amino]-phenyl]benzamide | | |
| methanesulfonate) | | |
| Interferon alfa-2a | ROFERON-A | Hoffmann-La Roche, |
| (recombinant peptide) | | Inc., Nutley, NJ |
| Interferon alfa-2b | INTRON A | Schering AG, Berlin, |
| (recombinant peptide) | (LYOPHILIZED | Germany |
| | BETASERON) | |
| Irinotecan HCl | CAMPTOSAR | Pharmacia & Upjohn |
| ((4S)-4,11-diethyl-4-hydroxy-9-[(4-piperi- | | Company |
| dinopiperidino)carbonyloxy]-1H-pyrano[3′, | | |
| 4′:6,7] indolizino[1,2-b] quinoline- | | |
| 3,14(4H, 12H) dione hydrochloride | | |
| trihydrate) | | |
| Letrozole | FEMARA | Novartis |
| (4,4′-(1H-1,2,4-Triazol-1-ylmethylene) | | |
| dibenzonitrile) | | |
| Leucovorin | WELLCOVORIN, | Immunex, Corp., |
| (L-Glutamic acid, N[4[[(2amino-5-formyl- | LEUCOVORIN | Seattle, WA |
| 1,4,5,6,7,8 hexahydro4oxo6- | | |
| pteridinyl)methyl]amino]benzoyl], calcium | | |
| salt (1:1)) | | |
| Levamisole HCl | ERGAMISOL | Janssen Research |
| ((−)-(S)-2,3,5,6-tetrahydro-6- | | Foundation, |
| phenylimidazo [2,1-b] thiazole | | Titusville, NJ |
| monohydrochloride C₁₁H₁₂N₂S•HCl) | | |
| Lomustine | CEENU | Bristol-Myers Squibb |
| (1-(2-chloro-ethyl)-3-cyclohexyl-1- | | |
| nitrosourea) | | |
| Meclorethamine, nitrogen mustard | MUSTARGEN | Merck |
| (2-chloro-N-(2-chloroethyl)-N- | | |
| methylethanamine hydrochloride) | | |
| Megestrol acetate | MEGACE | Bristol-Myers Squibb |
| 17α(acetyloxy)-6-methylpregna-4,6- | | |
| diene-3,20-dione | | |
| Melphalan, L-PAM | ALKERAN | GlaxoSmithKline |
| (4-[bis(2-chloroethyl) amino]-L- | | |
| phenylalanine) | | |
| Mercaptopurine, 6-MP | PURINETHOL | GlaxoSmithKline |
| (1,7-dihydro-6 H-purine-6-thione | | |
| monohydrate) | | |
| Mesna | MESNEX | Asta Medica |
| (sodium 2-mercaptoethane sulfonate) | | |
| Methotrexate | METHOTREXATE | Lederle Laboratories |
| (N-[4-[[(2,4-diamino-6- | | |
| pteridinyl)methyl]methylamino]benzoyl]- | | |
| L-glutamic acid) | | |
| Methoxsalen | UVADEX | Therakos, Inc., Way |
| (9-methoxy-7H-furo[3,2-g][1]-benzopyran- | | Exton, Pa |
| 7-one) | | |
| Mitomycin C | MUTAMYCIN | Bristol-Myers Squibb |
| mitomycin C | MITOZYTREX | SuperGen, Inc., |
| | | Dublin, CA |
| Mitotane | LYSODREN | Bristol-Myers Squibb |
| (1,1-dichloro-2-(o-chlorophenyl)-2-(p- | | |
| chlorophenyl) ethane) | | |
| Mitoxantrone | NOVANTRONE | Immunex |
| (1,4-dihydroxy-5,8-bis[[2-[(2- | | Corporation |
| hydroxyethyl)amino]ethyl]amino]-9,10- | | |
| anthracenedione dihydrochloride) | | |
| Nandrolone phenpropionate | DURABOLIN-50 | Organon, Inc., West |
| | | Orange, NJ |
| Nofetumomab | VERLUMA | Boehringer Ingelheim |
| | | Pharma KG, |
| | | Germany |
| Oprelvekin | NEUMEGA | Genetics Institute, |
| (IL-11) | | Inc., Alexandria, VA |
| Oxaliplatin | ELOXATIN | Sanofi Synthelabo, |
| (cis-[(1R,2R)-1,2-cyclohexanediamine- | | Inc., NY, NY |
| N,N'] [oxalato(2-)-O,O'] platinum) | | |
| Paclitaxel | TAXOL | Bristol-Myers Squibb |
| (5β,20-Epoxy-1,2a,4,7β,10β,13a- | | |
| hexahydroxytax-11-en-9-one 4,10-diacetate | | |
| 2-benzoate 13-ester with (2R,3 S)-N- | | |
| benzoyl-3-phenylisoserine) | | |
| Pamidronate | AREDIA | Novartis |
| (phosphonic acid (3-amino-1- | | |
| hydroxypropylidene) bis-, disodium salt, | | |
| pentahydrate, (APD)) | | |
| Pegademase | ADAGEN | Enzon |
| ((monomethoxypolyethylene glycol | (PEGADEMASE | Pharmaceuticals, Inc., |
| succinimidyl) 11-17-adenosine | BOVINE) | Bridgewater, NJ |
| deaminase) | | |
| Pegaspargase | ONCASPAR | Enzon |
| (monomethoxypolyethylene glycol | | |
| succinimidyl L-asparaginase) | | |
| Pegfilgrastim | NEULASTA | Amgen, Inc |
| (covalent conjugate of recombinant | | |
| methionyl human G-CSF (Filgrastim) and | | |
| monomethoxypolyethylene glycol) | | |
| Pentostatin | NIPENT | Parke-Davis |
| | | Pharmaceutical Co., |
| | | Rockville, MD |
| Pipobroman | VERCYTE | Abbott Laboratories, |
| | | Abbott Park, IL |
| Plicamycin, Mithramycin | MITHRACIN | Pfizer, Inc., NY, NY |
| (antibiotic produced by Streptomyces | | |
| plicatus) | | |
| Porfimer sodium | PHOTOFRIN | QLT |
| | | Phototherapeutics, |
| | | Inc., Vancouver, |
| | | Canada |
| Procarbazine | MATULANE | Sigma Tau |
| (N-isopropyl-μ-(2-methylhydrazino)-p- | | Pharmaceuticals, Inc., |
| toluamide monohydrochloride) | | Gaithersburg, MD |
| Quinacrine | ATABRINE | Abbott Labs |
| (6-chloro-9-(1-methyl-4-diethyl-amine) | | |
| butylamino-2-methoxyacridine) | | |
| Rasburicase | ELITEK | Sanofi-Synthelabo, |
| (recombinant peptide) | | Inc., |
| Rituximab | RITUXAN | Genentech, Inc., |
| (recombinant anti-CD20 antibody) | | South San Francisco, |
| | | CA |
| Sargramostim | PROKINE | Immunex Corp |
| (recombinant peptide) | | |
| Streptozocin | ZANOSAR | Pharmacia & Upjohn |
| (streptozocin 2-deoxy-2- | | Company |
| [[(methylnitrosoamino)carbonyl]amino]- | | |
| a(and b)-D-glucopyranose and 220 mg | | |
| citric acid anhydrous) | | |
| Talc | SCLEROSOL | Bryan, Corp., |
| (Mg₃Si₄O₁₀(OH)₂) | | Woburn, MA |
| Tamoxifen | NOLVADEX | AstraZeneca |
| ((Z)2-[4-(1,2-diphenyl-1-butenyl) | | Pharmaceuticals |
| phenoxy]-N,N-dimethylethanamine 2- | | |
| hydroxy-1,2,3-propanetricarboxylate (1:1)) | | |
| Temozolomide | TEMODAR | Schering |
| (3,4-dihydro-3-methyl-4-oxoimidazo[5,1- | | |
| d]-as-tetrazine-8-carboxamide) | | |
| Teniposide, VM-26 | VUMON | Bristol-Myers Squibb |
| (4′-demethylepipodophyllotoxin 9-[4,6-0- | | |
| (R)-2-thenylidene-(beta)-D- | | |
| glucopyranoside]) | | |
| Testolactone | TESLAC | Bristol-Myers Squibb |
| (13-hydroxy-3-oxo-13,17-secoandrosta-1,4- | | |
| dien-17-oic acid [dgr]-lactone) | | |
| Thioguanine, 6-TG | THIOGUANINE | GlaxoSmithKline |
| (2-amino-1,7-dihydro-6 H-purine-6- | | |
| thione) | | |
| Thiotepa | THIOPLEX | Immunex |
| (Aziridine, 1,1′,1″- | | Corporation |
| phosphinothioylidynetris-, or Tris (1- | | |
| aziridinyl) phosphine sulfide) | | |
| Topotecan HCl | HYCAMTIN | GlaxoSmithKline |
| ((S)-10-[(dimethylamino) methyl]-4-ethyl- | | |
| 4,9-dihydroxy-1H-pyrano[3′,4:′6,7] | | |
| indolizino [1,2-b] quinoline-3,14- | | |
| (4H,12H)-dione monohydrochloride) | | |
| Toremifene | FARESTON | Roberts |
| (2-(p-[(Z)-4-chloro-1,2-diphenyl-1- | | Pharmaceutical |
| butenyl]-phenoxy)-N,N- | | Corp., Eatontown, NJ |
| dimethylethylamine citrate (1:1)) | | |
| Tositumomab, I 131 Tositumomab | BEXXAR | Corixa Corp., Seattle, |
| (recombinant murine immunotherapeutic | | WA |
| monoclonal IgG₂ₐ lambda anti-CD20 | | |
| antibody (I 131 is a | | |
| radioimmunotherapeutic antibody)) | | |
| Trastuzumab | HERCEPTIN | Genentech, Inc |
| (recombinant monoclonal IgG₁ kappa anti- | | |
| HER2 antibody) | | |
| Tretinoin, ATRA | VESANOID | Roche |
| (all-trans retinoic acid) | | |
| Uracil Mustard | URACIL MUSTARD | Roberts Labs |
| | CAPSULES | |
| Valrubicin, N-trifluoroacetyladriamycin- | VALSTAR | Anthra --> Medeva |
| 14-valerate | | |
| ((2S-cis)-2-[1,2,3,4,6,11-hexahydro- | | |
| 2,5,12-trihydroxy-7 methoxy-6,11-dioxo- | | |
| [[4 2,3,6-trideoxy-3-[(trifluoroacetyl)- | | |
| amino-α-L-lyxo-hexopyranosyl]oxyl]-2- | | |
| naphthacenyl]-2-oxoethyl pentanoate) | | |
| Vinblastine, Leurocristine | VELBAN | Eli Lilly |
| (C₄₆H₅₆N₄O₁₀•H₂SO₄) | | |
| Vincristine | ONCOVIN | Eli Lilly |
| (C₄₆H₅₆N₄O₁₀•H₂SO₄) | | |
| Vinorelbine | NAVELBINE | GlaxoSmithKline |
| (3′,4′-didehydro-4′-deoxy-C′- | | |
| norvincaleukoblastine [R-(R*,R*)-2,3- | | |
| dihydroxybutanedioate (1:2)(salt)]) | | |
| Zoledronate, Zoledronic acid | ZOMETA | Novartis |
| ((1-Hydroxy-2-imidazol-1-yl- | | |
| phosphonoethyl) phosphonic acid | | |
| monohydrate) | | |

In some embodiments, the present invention provides pharmaceutical compositions of arsenic- platinum-loaded liposomal nano-particles, which may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Compositions and formulations for oral administration include powders or granules, suspensions or solutions in water or non-aqueous media, capsules, sachets or tablets. Thickeners, flavoring agents, diluents, emulsifiers, dispersing aids or binders may be desirable. Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients. Pharmaceutical compositions of the present invention include, but are not limited to, solutions, emulsions, and additional liposome-containing formulations. These compositions may be generated from a variety of components that include, but are not limited to, preformed liquids, self-emulsifying solids and self-emulsifying semisolids. The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product. The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Suspensions may further contain substances that increase the viscosity of the suspension including, for example, sodium carboxymethylcellulose, sorbitol and/or dextran. The suspension may also contain stabilizers. In one embodiment of the present invention the pharmaceutical compositions may be formulated and used as foams. Pharmaceutical foams include formulations such as, but not limited to, emulsions, microemulsions, creams, jellies and liposomes.

### Arsenic in Medicine

Arsenic was first used by Greek and Chinese healers more than 2,000 years ago to treat various diseases from syphilis to cancers. Arsenic-containing drugs played a central role in the development of modern pharmacology. In the late eighteenth century, Fowler's solution (a solution containing 1% potassium arsenite) was originally used to treat periodic fever, and later, a large variety of diseases including chronic myelogenous leukemia (CML) (Haller, J. S. Pharm. Hist. (1975) 17: 87-100). In 1910, Salvarsan (Arsphenamine), an organic arsenic-based drug, was disclosed to be effective in treating tuberculosis and syphilis (Ehrlich, P., Bertheim, A. U.S. Pat. No. 986,148 (1911)). Other organic arsenicals, such as Malarsoprol, are still used today to treat trypanosomiasis (an advanced sleeping sickness) (Dhubhghaill, O. M. N. et al. Structure and Bonding (1991) 78: 129-190).

In traditional Chinese medicine, arsenous acid or arsenic trioxide paste has been used to treat tooth marrow diseases, psoriasis, syphilis and rheumatosis. In the 1970's, arsenic trioxide was applied to treat acute promyelocytic leukemia (APL) in China (Sun, H. D. et al. Chin. J. Integrat. Trad. Clin. West. Med. (1992) 12: 170-171; Mervis, J. Science (1996) 273: 578; Chen, G.-Q. Blood (1996) 88: 1052-1061). Arsenic trioxide (TRISONEX) is now in phase III clinical trials for various leukemias including Acute Promyelocytic Leukemia (APL) and in phase I/II for relapsed/refractory multiple myeloma (MM) in China, Japan, Europe and the United States (Soignet, S. L. et al. N. Engl. J. Med. (1998) 339: 1341-1348; Jia, P. et al. Chin. Med. J. (2001) 114:19-24). Owing to a synergistic effect with retinoic acid, arsenic trioxide is often combined with retinoic acid for improved treatment of APL and MM.

Mineral forms of tetra-arsenic tetrasulfide (As₄S₄) and diarsenic trisulfide (As₂S₃), have been major components in other traditional medicines in China for more than 1500 years, such as realgar and orpiment. Recently, both As₄S₄ and As₂S₃ have been used in clinical trials in China for treatment of APL (Lu, D. et al. International Journal of Hematology (2002) 76: 316-318). The salts of arsenous acid, such as sodium arsenite (NaAsO₂), potassium arsenite (KAsO₂), calcium arsenite (Ca(HAsO₃)), copper arsenite (CuHAsO_{3,} Scheele's green), copper acetoarsenite (3Cu(AsO₂)₂.Cu(O₂CCH₃)_{2,} Paris green), and lead arsenite (Pb(HAsO₃)), and the salts of arsenic acid, such as calcium arsenate (Ca₃(AsO₄)_{2,} and lead arsenate (Pb₃(AsO₄)₂) are poisonous. They have been used as anticancer agents (sodium arsenite and potassium arsenite) and in viticulture as insecticides, weed killers, germicide and rodenticides, in preserving hides and in the manufacturer of soap and antiseptics (The Merck Index, 10^{th}, 1983; Columbia Encyclopedia, 6^{th}, 2004).

Despite its excellent therapy, arsenic compounds have a variety of widely appreciated toxic effects, including reduced viability of reticulo-endothelial cells (Roboz, G. J. et al. Blood (2000) 96: 1525-1530). Give this toxicity arsenic drugs must be given at low concentrations, which are ineffective in the treatment of many cancers. There is a need for methods for reducing the toxic side effects of arsenic while retaining its valuable therapeutic effect.

### Liposomes as Drug Carriers

Liposomes are microscopic lipid bilayer vesicles and have been widely used as carriers for a variety of agents such as drugs, cosmetics, diagnostic reagents, and genetic materials (New, R. Liposomes—a practical approach. Oxford University Press. 1990). Liposomes can encapsulate water-soluble agents in their aqueous cavities, or carry lipid-soluble agents within the membrane itself. Encapsulation of pharmaceuticals in liposomes can reduce drug side effects, improve pharmacokinetics of delivery to a target site, and improve the therapeutic index of a drug. Loading of drugs into liposomes is an important step in the development of drug delivery methods. Achieving maximum amount of drug accumulation inside liposomes, improving stability, reducing leakage, and realization of biocompatible-triggered release of drugs are major long-term goals. The loading methods vary depending on both physical and chemical properties of the drugs. In general, lipid-soluble drugs are easier to load because they easily incorporate into the lipid bilayer during liposome formation. Water-soluble drugs are also readily loaded because they interact with the polar head group of phospholipids facing the interior of liposomes and are therefore sequestered inside the liposomes. Amphiphatic compounds, on the other hand, are the most difficult to retain inside liposomes, as they can rapidly permeate through lipid bilayers.

The simplest method of drug loading is a passive entrapment of drugs in liposomes by hydration of the dry lipid film in an aqueous drug solution (Mayer, L. D. et al. Chem. Phys. Lipids (1986) 40: 333-345). The loading efficiency depends on the permeability of the drug across the membrane or the ease of the drug to escape from liposomes. This method is suitable for water-soluble drugs but not lipid-soluble ones.

For amphiphatic drugs, such as Doxorubicin (DXR), the previously reported-encapsulation method is loading of the drug into liposomes in response to a pH gradient where the internal pH of the liposome is made lower than the external medium pH and drugs consequently diffuse into liposomes in their neutral forms and are entrapped inside as positively charged forms (Mayer, L. D. et al. Biochim. Biophys. Acta (1986) 857: 123-126; (1990) 1025: 143-151). This method appears to be reasonably efficient for loading, if not for the fact that it requires internal acidification and external concentration of strong base (KOH), both of which cause lipid hydrolysis. Also, the resulting liposome-drug vesicles are unstable. Stable entrapment of DXR has been later reported where ammonium sulfate was used as the intraliposomal medium and DXR consequently entered and formed an aggregated form with sulfate and was encapsulated inside liposomes (Haran, G. et al. Biochim. Biophys. Acta (1993) 1151: 201-215). This method has enabled the clinical use of DXR-loaded sterically-stabilized liposomes. It is today called DOXIL (doxorubicin HCl liposome injection). DOXIL has been approved for the treatment of AIDS-related Kaposi's sarcoma (the U.S. Food and Drug Administration, 1995), refractory ovarian cancer (the U.S. Food and Drug Administration, 1999), metastatic breast cancer in combination with cyclophosphamide (Europe, 2000), and refractory breast cancer (Europe and Canada, 2003) (Allen, T. M. et al. Science (2004) 303: 1818-1822). Cisplatin (cis-diamminedichloroplatinum) is an anticancer drug used worldwide in the treatment of epithelial malignancies such as lung, head and neck, ovarian and testicular cancer. The approach of preparing less toxic, liposomal formulations has been studied that use a passive method of encapsulating cisplatin in liposomes by hydration of the dry lipid film in the cisplatin aqueous solution (Yatvin, M. B. et al. Cancer Res. (1981) 41: 1602-1607; Steerenberg, P. A. et al. Cancer Chemother. Pharmacol. (1988) 21: 299-307). Due to both the low water solubility and low lipophilicity of cisplatin, this method provides very low encapsulation efficiencies with a very low drug-to-lipid ratio which limits the bioavailability of cisplatin in the tumor and results in low cytoxicity (Bandak, S. et al. Anticancer drugs (1999) 10: 911-920; Newman, M. S. et al. Cancer Chemother. Pharmacol. (1999) 43: 1-7). Recently, a new method was developed by combining negatively charged phospholipids, such as 50% phosphatidylserine (PS), into the neutral phosphatidylcholine (PC). The negative head groups of PS lipids appear to interact with the positively charged aqualized-species of [(NH₃)₂Pt(H₂O)₂]²⁺ and allow for efficient and stable aggregates of {(NH₃)₂Pt}²⁺ within liposomes, leading to high cytotoxicity (Burger, K. N. J. et al. Nature Medicine (2002) 8: 81-84).

There is high demand for novel arsenic-based drugs that exhibit higher activities but lower toxic side effects than the solution of the mineral compounds. This can be realized by means of a lipid coating. Arsenic trioxide is an amphiphatic agent (soluble both in aqueous and hydrophobic phases), which makes liposomal formulation difficult. Previous attempts to prepare liposomal arsenic trioxide by hydration of lipid components in the concentrated aqueous solution of arsenic trioxide (Kallinteri, P. et al. J. Liposome Res. (2004) 14: 27-38) were met with limited success; the resulting Liposome-arsenic vesicles were unstable and suffered from substantial leakage of the drug within 24 hours. This significantly impaired the application of this method. The present invention provides an unprecedented approach for loading arsenic drugs into liposomes and delivery of arsenic into specific tumor cells allowing for useful pharmaceutical preparations comprising liposomes containing arsenic drugs. The present invention creates a novel system that takes advantage of transmembrane gradients of transitional metal ion salts to obtain the efficient and stable loading of a weak acid-H₃AsO₃ into liposomes by forming nano-particles inside (miniralization). The formation of insoluble metal-arsenite complexes and the efflux of acetic acids (HAc) are the two driving forces for the efficient accumulation of arsenic inside liposomes. Both metal cation and anion (e.g., acetate, famate, lactate and hydroxyacetate) have important roles in drug loading and release.

In some embodiments, the present invention provides methods for loading arsenic into liposomes, comprising: preparing liposomes comprising an encapsulated metal ion and adding an agent such as arsenite, arsenic trioxide, arsenic sulfide, arsenate, methylarsinic acid and dimethylarsinic acid and other arsenic analogues.

The present invention further provides methods of synthesis of several new compositions of matter. In some embodiments, they are liposomes comprising Mₙ(AsX₃)ₘ particles, where X=O, OH, S, SH, Se, SeH; M=metal ion; n=1, 2, 3; m=1, 2, 3. Methods include selecting a metal cation or an anion for encapsulation in a liposome to achieve desired retention of an encapsulated agent. The efficiency and stability of loading and release of drugs can be modified and controlled by employing different cations and anions. Screening for activity can be conducted to select optimized conditions as desired.

The metal ions for use in this invention include, but are not limited to, transitional metals of the group 1B, 2B, 3B, 4B, 5B, 6B, 7B and 8B elements (groups 3-12), and the basic metals from groups of IIIA and IVA and VA. Preferred metals may be selected from one or more of Ni, Co, Cu, Zn, Mn, Fe, Pb, V, Ti, Cr, Pt, Rh, Ru, Mo, Hg, Ag, Gd, Cd and Pd. The metal ions may include their radical reactive isotopes, such as ⁷¹As, ⁷²As, ⁷³As, ⁷⁴As, ⁷⁶As, ⁷⁷As, ⁵⁷Ni, ⁶⁵Ni, ⁶⁶Ni, ⁵⁵Co, ⁶¹Cu, ⁶²Cu, ⁶⁴Cu, ⁶⁶Cu, ⁶⁷Cu, ⁷²Zn, ⁵¹Mn, ^{52m}Mn, ⁹⁹Mo, ^{99m}Tc, ²⁰³Pb, ⁶³Ga, ⁶⁶Ga, ⁶⁷Ga, ¹¹¹In, ⁹⁷Ru, ⁵²Fe, ⁵¹Cr, ¹⁸⁶Re, ¹⁸⁸Re, ⁹⁰Y, ¹⁶⁹Er, ^{117m}Sn, ¹²¹Sn, ¹²⁷Te, ¹⁴²Pr, ¹⁴³Pr, ¹⁹⁸Au, ¹⁹⁹Au, ¹⁴⁹Tb, ¹⁶¹Tb, ¹⁰⁹Pd, ¹⁶⁵Dy, ¹⁴³Pm, ¹⁵¹Pm, ¹⁵⁷Gd, ¹⁶⁶Ho, ¹⁷²Tm, ¹⁶⁹Yb, ¹⁷⁵Yb, ¹⁷⁷Lu, ¹⁰⁵Rh, ¹¹¹Ag, ⁸⁹Zr, ^{82m}Rb, ¹¹⁸Sb, ^{193m}Pt, ^{195m}Pt. This present invention is not limited to the encapsulation of arsenic compounds. The encapsulation methods of the present invention further are applicable to the encapsulation of other bioactive agents for the therapy or diagnosis of disease.

The present invention provides encapsulation methods that are applicable for other amphiphatic agents. Preferably, a therapeutic agent is one that is able to diffuse across lipid- or polymer- membranes at a reasonable rate and which is capable of coordinating with a metal encapsulated within the liposome in a prior step. Agents that are capable of coordination with a transition metal typically comprise of coordination sites such as hydroxyl, thiols, acetylenes, amines or other suitable groups capable of donating electrons to the transition metal thereby forming a complex with the metal.

The present invention provides encapsulation methods that are applicable for multi-drug co-encapsulation into one vesicle, provided that one or more therapeutic agents are first passively encapsulated inside liposomes and the second therapeutic agent is added to the external solution of said liposomes and is thus actively loaded. Two or more drugs, such as inorganic drugs of arsenic, cisplatin (cis-diaminedichloroplatinum) and its hydrolyzed products, and tetrathiomolybate and its hydrolyzed products, and organic drugs of retinoic acid and nucleoside analogues, 8-chloro- or 8-NH₂-adenosine, etc. can be incorporated into liposomes by combining passive and active methods of loading.

The novel liposomal Mₙ(AsX₃)ₘ nano-particles of the present invention have a long shelf life (e.g., greater than a day, week, month, 6 months, year, etc.). This meets the pharmaceutical requirements for clinical use. No “bedside” preparation of liposomal arsenic drugs is required immediately before patient treatment and the formulation is ready for injection. The novel Mₙ(AsX₃)ₘ nano-particles of the present invention have a specialized feature: they will dissolve in low pH environments, like those found within compartments of cancerous cells. The arsenic release from liposomal Mₙ(AsX₃)ₘ nano-particles is triggered by lowering pH values. The accurate controlled release of arsenic can be realized by making use of different degree of acidic sensitivity of different Mₙ(AsX₃)ₘ complexes.

The present invention also provides methods of arsenic release, either triggered by temperature, pH or by employing liposomes comprised of the fluid lipids with lower gel-to-crystal transitional temperatures (Tₘ), such as dioleoylphosphatidylcholine (DOPC) (Tₘ=−20° C.), dioleoylphosphatidylglycerol (DOPG) (Tₘ=−18° C.), palmitoyl-oleoyl-phosphatidylcholine POPC (Tₘ=−2° C.), dilauroyl-phosphatidylcholine (DLPC) (Tₘ=−1° C.), dimyristoyl-phosphatidylcholine (DMPC) (Tₘ=23° C.), egg-phosphatidylcoline egg-PC (Tₘ=37° C.).

The present invention further provides a method of preparation of arsenic-encapsulated liposomes with a broad spectrum of types, sizes, and composition, including sterically-stabilized liposomes, immunoliposomes, and sterically-stabilized immunoliposomes. The encapsules can be all types of vesicles, such as liposomes, lipid emulsions, micelles, and nano- or micro-spheres.

The present invention also provides methods of coupling liposomal Mₙ(AsX₃)ₘ nano-particles to antibodies, such as Rituxan, and of evaluating cytotoxicity of conjugates on the human B-cell lymphoma SU-SHL-4. Such ligand-targeted liposomal Mₙ(AsX₃)ₘ are effective therapeutics and exhibit lower toxicity as compared with the parent arsenic drugs.

The targeting antibodies applicable to this invention can be various types of antibodies, including, but not limited to, Rituxan, Campath-1H, HM1.24, HER2, Anti-CD38, HuM195, HP67.6. Non-antibody ligands include, for example, including, but not limited to, folate, retinoic acid, estrogen analogs such as galactosamine, Arg-Gly-Asp tripeptide (RGD), Asn-Gly-Arg (NGR), Octreotide, Granulocyte-macrophage colony-stimulating factor (GM-CSF), and proteins, such as transferrin are also suitable for use with the present invention.

The present invention further provides a method of preparing and using ligand-targeted liposomal Mₙ(AsX₃)ₘ for treatment of various types of tumors, including, but not limited to, hematological tumors, such as Lymphoma, Multiple Myeloma (MM), Acute Promyelocytic Leukemia (APL), Acute Myeloid Leukemia (AML), Chronic Lymphocytic Leukemia (CLL), and solid tumors, such as breast, ovarian, pancreate, bladder, lung, liver, brain, neck, colorectal cancers, etc.

### Lipid-Drug Complexes

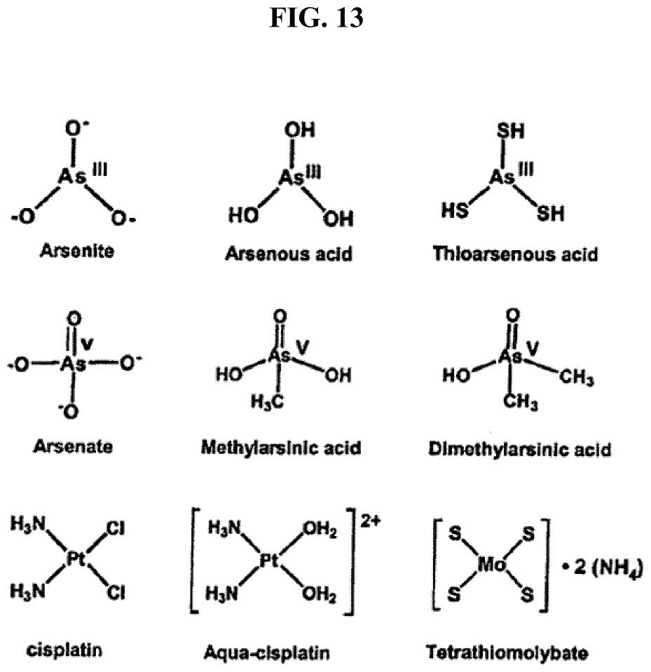

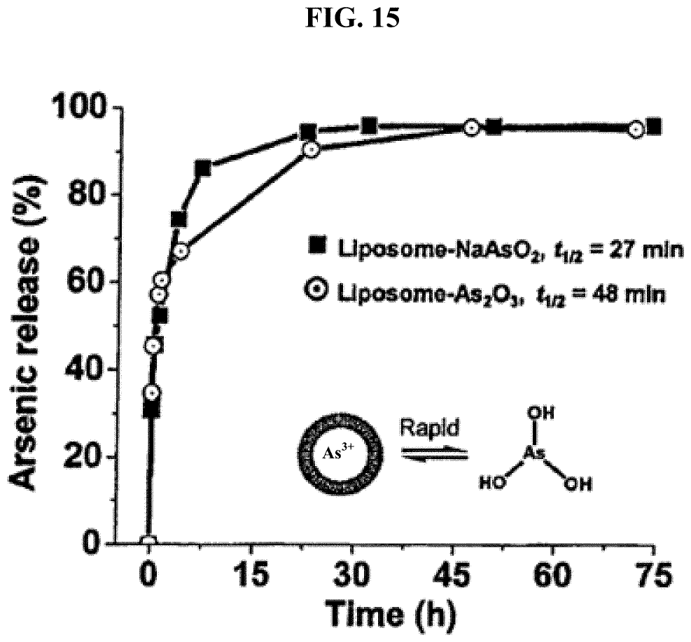

In neutral or acidic solutions, arsenic(III) species (FIG. 13) are primarily present as neutral H₃AsO₃ due to the pKₐ of 9.3 for H₂AsO₃²⁻ (Loehr, T. M. et al. Inorg. Chem. (1968) 7: 1708-1714). H₃AsO₃ is soluble both in aqueous and hydrophobic phases and readily diffuses across the lipid membrane (Example 9 and FIG. 15). The passively encapsulated H₃AsO₃ (150 mM) leaks out after 24 h at 4° C. with half-time <50 min (FIG. 15). It is difficult to realize the stable retention and controlled release of arsenic using passive methods in the encapsulation of arsenic(III) drug under physiological conditions.

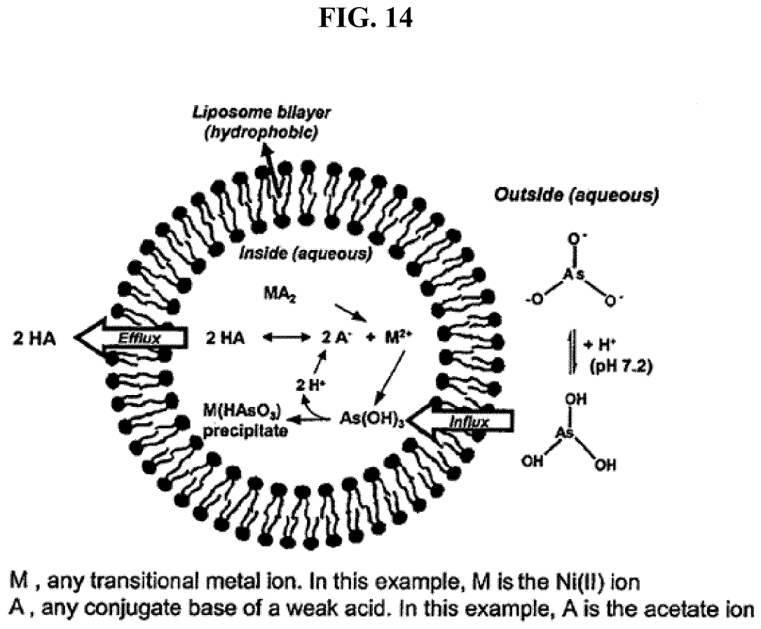

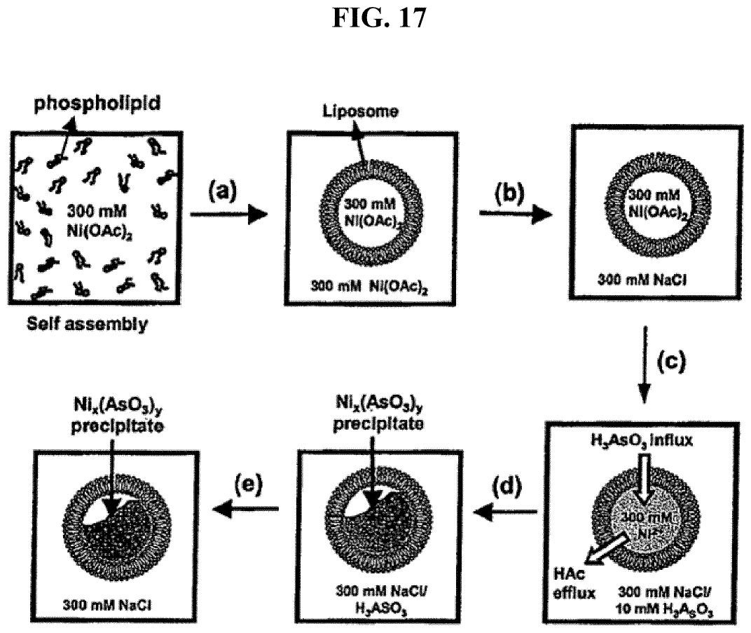

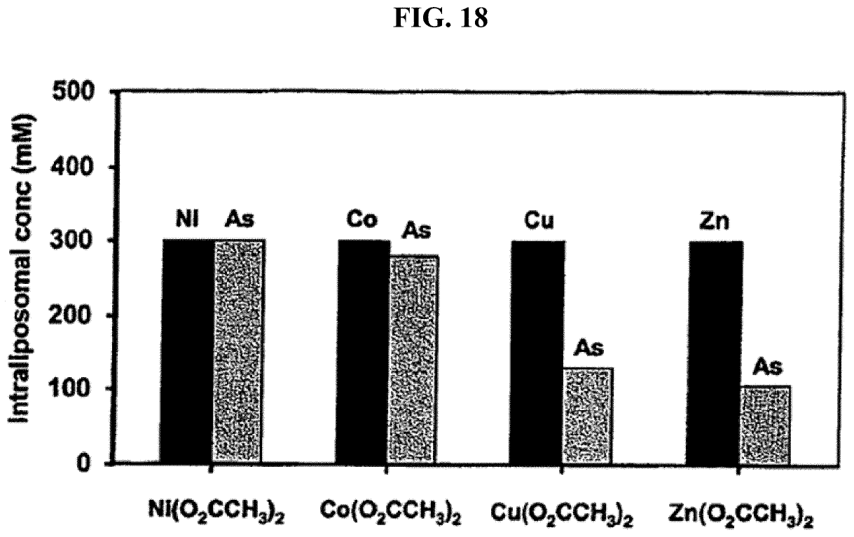

Based on the findings herein that arsenite could form both aqueous and hydrophobic insoluble complexes with transitional metal ions, such as Ni²⁺, Co²⁺, Cu²⁺, Zn²⁺, Mn²⁺, Fe²⁺, and Pb²⁺ at neutral pH, and that such complexes are acid-sensitive and will re-dissolve when lowering pH values, an efficient system for encapsulating arsenic (III) drugs into liposomes has been created (see e.g., Example 10 and FIGS. 14, 16 and 17). An embodiment of this method involves first passively encapsulating a certain concentrated metal salt, such as 300 mM Ni(O₂CCH₃)_{2,} inside the liposomes, and removing the extraliposomal metal salt to create a gradient between the internal and external aqueous phase of liposomes (see the procedure in FIG. 17). This is followed by the addition of NaAsO₂ or As₂O₃ at pH 7.2, resulting in the active loading of arsenic(III) into liposomes with a half-life <5 min at 50° C. and with a final arsenic accumulation up to 300 mM within one 100-nm liposome vesicle (FIG. 18). This indicates that a single 100-nm liposome can carry greater than 90,000 arsenic molecules.

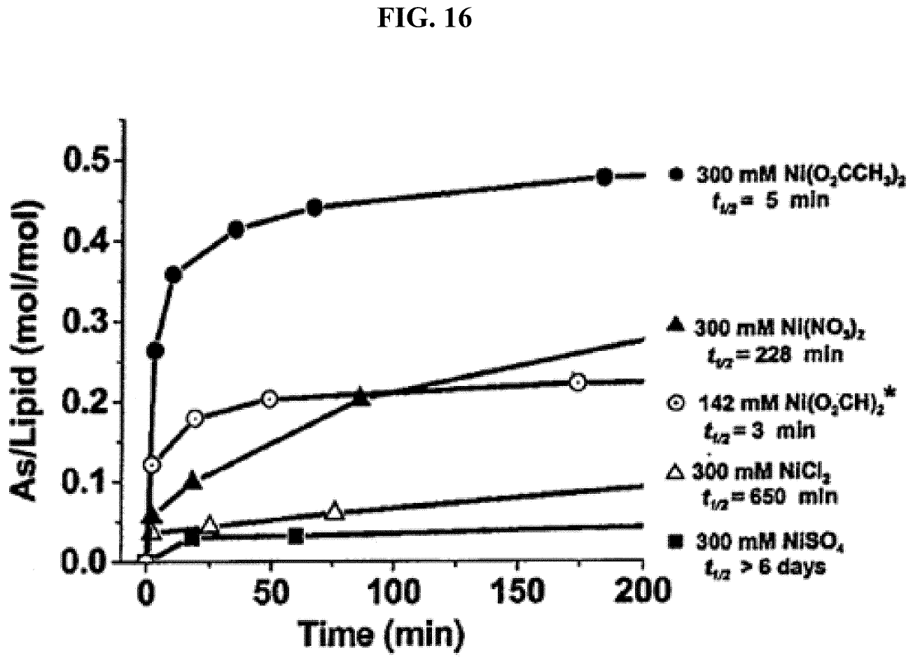

During a loading cycle, the external arsenite ions are protonated (at pH 7.2) to the neutral As(OH)₃ which diffuses across the lipid membrane to the internal liposome. By binding to Ni²⁺ to form the insoluble nickel(II) arsenite complexes inside, such as Ni(HAsO₃), As(OH)₃ releases two protons which bind to two acetate anions. The resulting acetic acids (HA) diffuse across the membrane to the external liposome in exchange for the influx of As(OH)₃, leading to significant accumulation of arsenic inside liposomes. Both the formation of insoluble nickel(II) arsenite complexes and the efflux of acetic acid are the driving forces for the arsenic uptake (FIG. 16). For this novel system, the Ni²⁺ part can be any other transitional metal ions, such as Co²⁺, Cu²⁺, Zn²⁺, Mn²⁺, Fe²⁺, and Pb²⁺, which are able to form insoluble complexes with arsenite (under similar experimental conditions, using 300 mM sodium acetate as intraliposomal medium resulted in little arsenic uptake). The salt of sodium arsenite is water-soluble. The Ac⁻ part can be any other anions ready to accept protons to form neutral compounds with lower molecular weight which can diffuse across the membrane rapidly, such as formate, lactate and hydroxyacetate (FIG. 16). The nature of the anion has significant influence on the efficiency of arsenic uptake as shown in Example 3 and FIG. 16. Under similar experimental conditions, the uptake rates are in the following order: 300 mM Ni(O₂CCH₃)₂≈142 mM Ni(O₂CH)₂>>300 mM Ni(NO₃)₂>300 mM NiCl₂>>300 mM NiSO₄. There is little arsenic uptake in the case of 300 mM NiSO₄. Compared with the pKₐ values of acetic acid (4.75), formic acid (3.75), HNO₃ (−2), HCl (−7), and H₂SO₄ (−10), and considering the lower molecular weight and versatile properties for acetic acid, formic acid, nitric acid, and hydrochloric acid but not for sulfuric acid, arsenic loading efficiency appears to be facilitated by the ability of anions to accept protons for forming the neutral compounds which are ready to efflux from liposomes (FIG. 14).

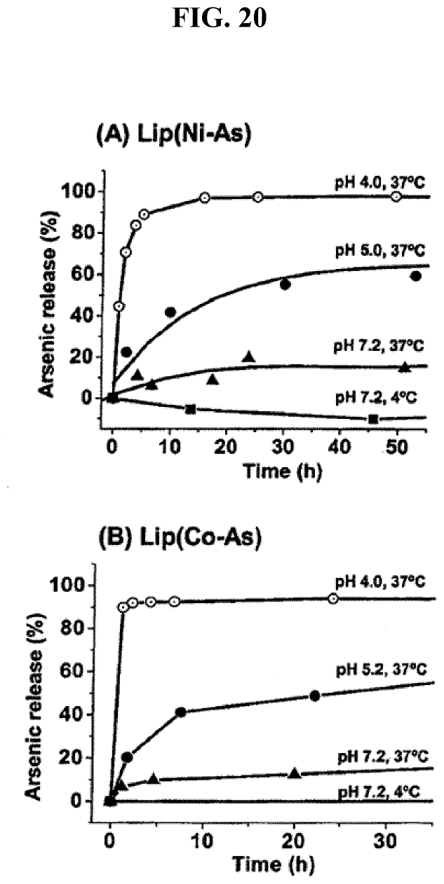

The active loading of arsenic using other acetate salts of M²⁺, such as Co²⁺, Cu²⁺, or Zn²⁺ has shown similar behaviors to that of Ni(O₂CCH₃)₂ (Examples 11-13 and FIGS. 18, 22 and 23), and achieved the rapid equilibrium with half time <10 min. The final extent of arsenic uptake is somewhat less in the cases of Cu²⁺ and Zn²⁺ with the As-to-lipid molar ratio of 0.4 and 0.2, respectively, when compared with the 0.5 and 0.6 As-to-liposome molar ratios for Ni² and Co²⁺ respectively (FIG. 18). Low uptake might be due to a less stable metal-arsenite complex, a different pH optimum for complex formation, and/ or membrane permeability of the complex. The novel liposomal Mₓ(AsO₃)_{y} nano-particles show long shelf life (>6 months at 4° C. for Lip(Ni—As) and Lip(Co—As), Example 14 and FIG. 20). This meets the pharmaceutical requirements for clinical use. No “bedside” preparation of liposomal arsenic drugs is required immediately before patient treatment and the formulation is ready for injection. Due to the acid sensitivity of Mₓ(AsO₃)_{y} complexes, the arsenic release is triggered by lowering pH values (Example 15 and FIG. 20). Lip(Co—As) particles are more acid-sensitive than Lip(Ni—As), which is consistent with the observation that the Coₓ(AsO₃)_{y} complex is almost completely dissolved when pH <5.5 while Niₓ(AsO₃)_{y} is completely dissolved when pH <4.0. The accurate controlled release of arsenic can be realized by making use of different degree of acidic sensitivity of arsenite complexes with different transitional metals.

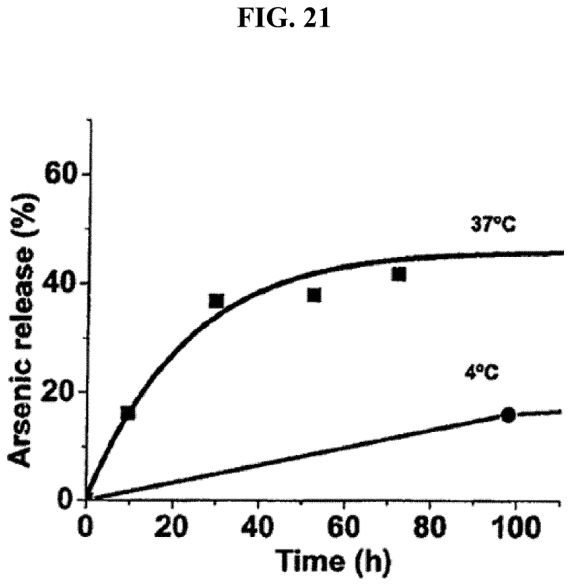

This drug loading system is also applicable to other types of liposomes. The more fluid lipids, such as dioleoylphosphatidylcholine (DOPC, with the gel-to-crystal transitional temperatures (Tₘ) of −20° C.) can be employed. Rapid and efficient uptake was achieved for liposomes with DOPC/dioleoylphosphatidylglycerol (DOPG)/Cholesterol (Chol) (65/5/30, wt %) as described in Example 16. FIG. 21 shows the stability for Niₓ(AsO₃)_{y} inside liposomes at pH 7.2 with 16% release after 100 h storage at 4° C. The release was ten times faster when the temperature was raised to 37° C. Efficient loading was also achieved for the liposomes functionalized by PEG-2000 and Maleimide, with a 0.33 As-to-lipid molar ratio (Example 17). This loading system permits the preparation of arsenic-encapsulated liposomes with a broad spectrum of types, sizes, and composition, including sterically-stabilized liposomes, immunoliposomes, and sterically-stabilized immunolipsomes.

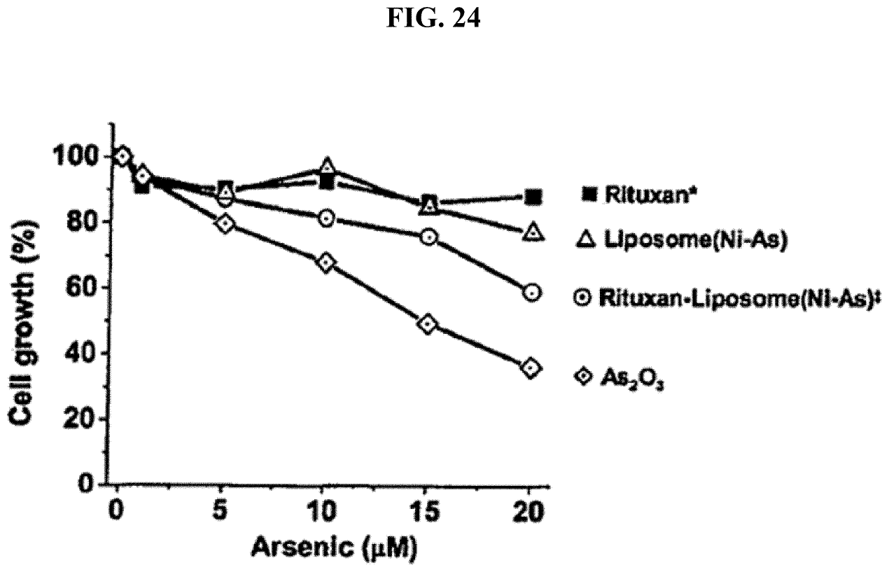

Monoclonal antibodies (mAb), such as anti-CD20 Rituxan, can be coupled to the Liposome(Ni—As) to provide the products of mAb-Lipsome(Ni—As) (Example 17). The cytotoxicities of Liposome(Ni—As) and Rituxan-Liposome(Ni—As) were tested on the human lymphoma cell line of SU-DHL-4 where CD20 antigens are expressed on the surface (Example 18). This was compared to the cytotoxicities of free As₂O₃ and Rituxan. It was found that when the cells were exposed to those drugs for a long period (three days at 37° C.) that most of encapsulated arsenic species were released from Liposome(Ni—As) (IC₅₀=1.92 uM) or Rituxan-Liposome(Ni—As) (IC₅₀=1.52 uM), and exhibited the killing ability as effective as the free As₂O₃ (IC₅₀=1.45 uM). When the cells were exposed to those drugs for a shorter time (20 min at 37° C.), the arsenic species were still sequestered inside liposomes, to be delivered to the tumor cell by the recognition of Rituxan to the CD20 on the cell surface. This is followed by release of arsenic for eradicating the tumor (FIG. 24).

### Treatment of Disease

The liposome encapsulated drugs of the present invention find use in the treatment of a variety of disease states. Exemplary diseases include, but are not limited to cancer (e.g., leukemia), autoimmune disease (e.g., psoriasis and rheumatoid arthritis), tuberculosis, and syphilis.

### Combination Therapy

In some embodiments, the compositions of the present invention are provided in combination with existing therapies. In other embodiments, two or more compounds of the present invention are provided in combination. In some embodiments, the compounds of the present invention are provided in combination with known cancer chemotherapy agents. The present invention is not limited to a particular chemotherapy agent.

Various classes of antineoplastic (e.g., anticancer) agents are contemplated for use in certain embodiments of the present invention. Anticancer agents suitable for use with the present invention include, but are not limited to, agents that induce apoptosis, agents that inhibit adenosine deaminase function, inhibit pyrimidine biosynthesis, inhibit purine ring biosynthesis, inhibit nucleotide interconversions, inhibit ribonucleotide reductase, inhibit thymidine monophosphate (TMP) synthesis, inhibit dihydrofolate reduction, inhibit DNA synthesis, form adducts with DNA, damage DNA, inhibit DNA repair, intercalate with DNA, deaminate asparagines, inhibit RNA synthesis, inhibit protein synthesis or stability, inhibit microtubule synthesis or function, and the like.

### Pharmaceutical Compositions

The present invention further provides pharmaceutical compositions (e.g., comprising the liposome encapsulated compounds described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids, etc.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Certain embodiments of the invention provide pharmaceutical compositions containing (a) one or more liposome encapsulated compounds of the present invention and (b) one or more other chemotherapeutic agents. Examples of such chemotherapeutic agents are described above. Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Other chemotherapeutic agents are also within the scope of this invention. Two or more combined compounds may be used together or sequentially.

### Anti-uPA Antibodies as Targeting Antibodies

In certain embodiments, an anti-uPA (anti-urokinase plasminogen activator) antibody is used as a targeting antibody for the liposomal nanoparticles described herein. In particular embodiments, the anti-uPA antibody targets the kringle region of uPA. In some embodiments, the anti-uPA antibody is the ATN-291 antibody described in U.S. Pat. Pub. No. 2005/0232924 (herein incorporated by reference). The ATN-291 antibody targets the urokinase plasminogen activator. The urokinase plasminogen activator (uPA) is a protein that is involved in the remodeling of tumor matrix and tumor cell invasion and is therefore thought to play an important role in tumor progression. uPA is over-expressed by most solid tumors evaluated to date and has been proposed a s a therapeutic target for the treatment of cancer. uPA binds to the surface of tumor cells via a specific receptor (uPAR) and under cetain conditions, the uPA-uPAR complex can be internalized (endocytosed). Monoclonal antibody ATN-291 targets the kringle domain of uPA and induces the internalization of uPA. The kringle domain of uPA is not involved in the binding of uPA to uPAR and thus ATN-291 can bind to uPA regardless of whether it is bound to uPAR or not. In certain embodiments, anti-uPA-nanoparticles of the present invention are employed to internalize into cells the contents of the nanoparticles (e.g., platinum and/or arsenic compounds). The uPA-nanoparticle conjugates therefore provide a more specific way of delivering cytotoxic agents to a tumor cell while spearing normal tissue, which does not express uPA.

### EXPERIMENTAL

### Example 1

### Compositions and Methods

Materials. Dipalmitoylphosphatidylcholine (DPPC), dioleoylphosphatidylglycerol (DOPG) and 1,2-Dipalmitoyl-sn-glycero-3-phosphoethanolamine-N-(Lissamine rhodamine B sulfonyl) (ammonium salt) (DPPE-Rh) were purchased from Avanti Polar Lipids (Alabaster, Ala., USA). 1,2-Distearoyl-sn-glycero-3-phosphoethanolamine-N-[folate(polyethylene glycol)-3350] (DSPE-PEG₃₃₅₀-Folate) was synthesized according to the literature (Gabizon et al. Bioconjug. Chem. 1999, 10, 289-298., herein incorporated by reference in its entirety). Cholesterol (Chol), arsenic trioxide (As₂O₃), sodium arsenite (NaAsO₂), cisplatin (cisPt), silver acetate (Ag(OAc)), folic acid (FA), paraformaldehyde, 2-[4-(2-hydroxyethyl)-1-piperazine]ethanesulfonic acid (HEPES), 2-[N-Morpholino]ethanesulfonic acid (MES), Bicine, sucrose, sodium dodecyl sulfate (SDS), phenazine methosulfate (PMS), human insulin solution, and Sephadex G50 were obtained from Sigma-Aldrich (St. Louis, Mo., USA). Sodium nitrate was from Mallinckrodt (Kentucky, USA). 3-(4,5-dimethylthiazol-2-yl)-5-(3-carboxymethoxyphenyl)-2-(4-sulfophenyl)-2H-tetrazolium (MTS) was from Promega (Madison, Wis., USA). RPMI-1640, folate-deficient RPMI-1640, fetal bovine serum (FBS) were from INVITROGEN-GIBCO (Carlsbad, CL, USA). Charcoal dextran—stripped fetal bovine serum (cds-FBS) was from Atlanta Biologicals, Inc. (Lawrenceville, Ga., USA). Eagle's Minimum Essential Medium (EMEM) was from the American Type Culture Collection (ATCC) (Manassas, Va., USA). L-Glutamine, penicillin-streptomycin, and phosphate-buffered saline (PBS) were from MEDIATECH (Herndon, Va., USA). Amphotericin B from Biologas (Montgomery, Ill., USA).

Preparation of lipid film. Lipid mixtures of DPPC/DOPG/Chol with various molar ratios (51.4/3.6/45, 86.4/3.6/10, and 96.4/3.6/0) and of DSPC/DSPE-PEG₂₀₀₀/Chol (51/4/45%) were dissolved in chloroform. For rhodamine (Rh)-labeled liposomes, 0.5% DPPE-Rh was included. The chloroform was then removed by rotary vacuum evaporation to form lipid film on the vial, which was subsequently placed under a high vacuum overnight to remove any residual solvent.

Preparation of aqua-cisplatin acetate. The acetate solution of aqua-cisPt (300 mM [cis-(NH₃)₂Pt(OH₂)₂](OAc)₂) was prepared as previously reported (Appleton et al. Inorg. Chem. 1984, 23, 3514-3521.). Briefly, 360 mg cisPt (cis-(NH₃)₂PtCl₂) was mixed with 370 mg Ag(OAc) in 4 mL MQ-H₂O at 50° C. in the dark for 4-5 h. The mixture was then kept at 20° C. overnight before filtered through a 0.2 μm syringe filter (cellulose acetate membrane, Nalgene) to remove the white AgCl precipitate. The pale yellow solution obtained (pH 5.1) was sealed and kept at 4° C. in the dark and used within two weeks.

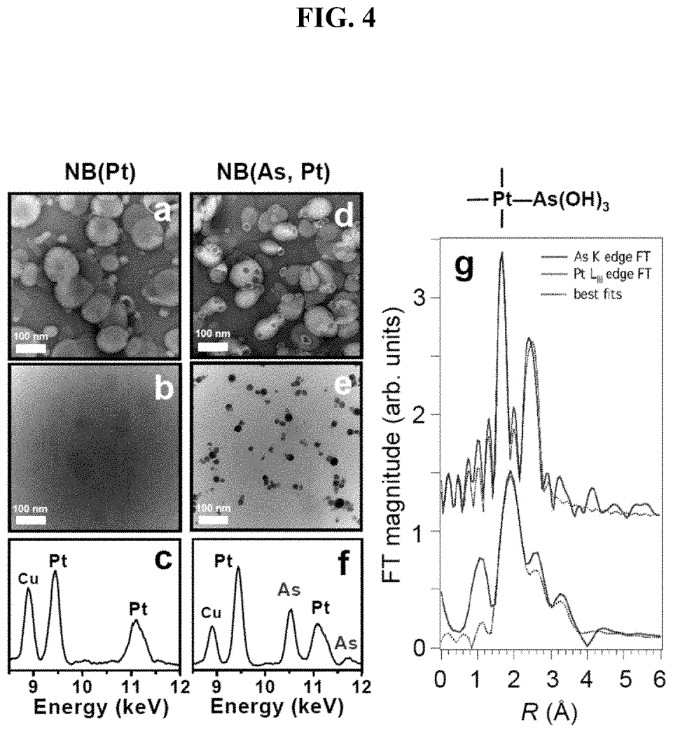

Transmission electron microscopy (TEM). Liposome samples were stained with 2% uranyl acetate on 400-mesh copper grids (carbon-coated and formvar-covered, Ted Pella, Inc., USA), and air-dried overnight before TEM analysis at 200 kV, magnification 40,000×(Hitachi HF2000, Hitachi High-Technologies, Japan). For visualization of the inorganic cores within the liposomes, some liposome samples were left unstained to avoid the influence of the electron density of uranyl acetate (Kallinteri et al. J. Liposome Res. 2004, 14, 27-38., Douglas & Young, Nature 1998, 393, 152-155., herein incorporated by reference in their entireties). The unstained samples were analyzed for arsenic and platinum components within the liposomal cores by energy-dispersive X-ray analysis (EDX) (SEE FIG. 4A-F).

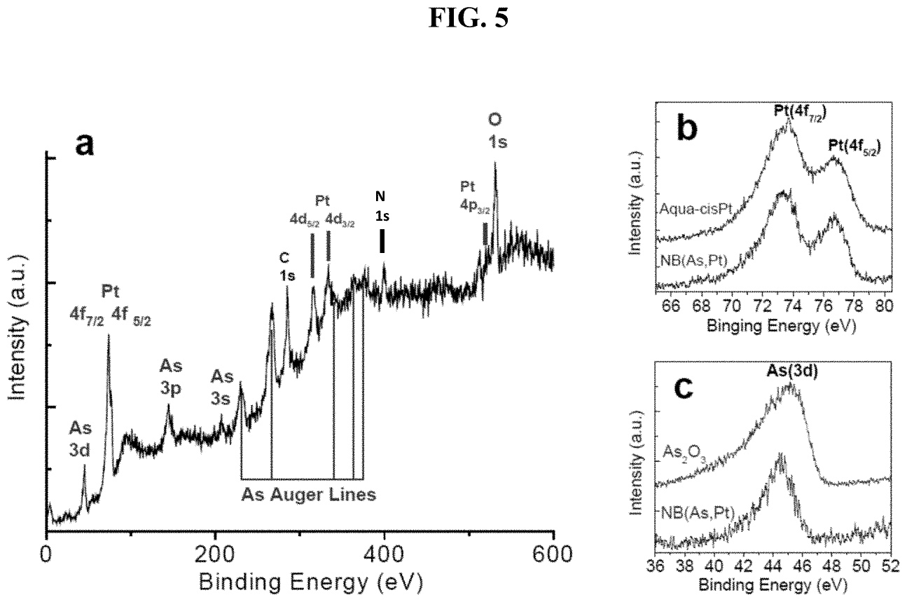

X-ray photoelectron spectroscopy (XPS). NB(As, Pt) sample was centrifuged (2100 g, 30 min, 20° C.) to collect liposome nanoparticles. The NB(As, Pt) pellet was washed ×3 by MQ-H₂O and then freeze-dried under high vacuum. The dried mixture was washed ×5 by chloroform for complete removal of the lipids and further dried under high vacuum and P₂O₅. The obtained complex(As, Pt)_{01.36} (As/Pt=1.36 molar ratio, pale-yellow, air-stable) was analyzed by X-ray photoelectron spectroscopy (XPS) under the Omicron ESCA Probe (Omicron Nanotechnology, Taunusstein, Germany) (SEE FIG. 5), and its results were compared with those of As₂O₃, NaAsO₂, cisPt, and aqua-cisPt acetate. All samples (powder) were embedded into adhesive carbon tapes and mounted in the analysis chamber. The spectra were acquired with X-ray illumination (beam energy 14 eV) under high vacuum (1.0×10⁻⁹ mbar). The surface charge was neutralized with the electron gun. The data were analyzed by the ESI software (Version 2.4, Omicon Nano Technology Ltd., Germany), using the Cls peak (284.8 eV) as calibration reference (Moulder et al. Handbook of X-ray Photoelectron Spectroscopy Physical Electronics, Inc., Minnesota, USA, 1995.).

X-ray absorption spectroscopy (XAS). Aqueous NB(As, Pt) samples were loaded into Lucite cuvettes with 40 μm Kapton windows and rapidly frozen in liquid nitrogen. As K-edge, Pt L_{II} and Pt L_{III} XAS data were collected at Stanford Synchrotron Radiation Laboratory (SSRL). X-ray absorption near-edge structure (XANES) data were normalized by fitting data to the McMaster absorption coefficients below and above the edge using a single background polynomial and scale factor (McMaster et al., 1969, (Commerce, U.S.D.o., Ed.)., Weng et al. J. Synchrotron Radiat. 2005, 12, 506-510., herein incorporated by reference in their entireties). The EXAFS background correction for both As K-edge and Pt L_{II} and L_{III}-edge was performed by fitting a three-region cubic spline for all samples. The data were then converted to k-space using E₀=11887 eV for As, and E₀=13292.3 eV for Pt. Fourier transforms were calculated using k³ weighted data over a ranges of 3.5-15.1 Å⁻¹ (for the As XAS data) and 2.6-10.5 Å⁻¹ (for the Pt XAS data) The program Feff version 7.02 (Zabinsky et al. Phys. Rev. B: Condens. Matter 1995, 52, 2995-3009., herein incorporated by reference in its entirety) was used to calculate amplitude and phase functions, Aₛ(k)exp(−2Ras/λ) and ϕₐₛ(k) for As—O and Pt—O/N interactions at 2.0 Å, and Pt—As and As—As interactions at 3.0 Å. Data were analyzed in k-space using the program EXAFSPAK (George & Pickering, 1993, Stanford University, Palo Alto, Calif., herein incorporated by reference in its entirety). For all data, Sₛ was fixed at 0.9 based on fits to the EXAFS data for structurally characterized model complexes (SEE FIG. 4G) (McClure et al. J. Inorg. Biochem. 2003, 94, 78-85., Clark-Baldwin et al. J. Am. Chem. Soc. 1998, 120, 8401-8409., herein incorporated by reference in their entireties).

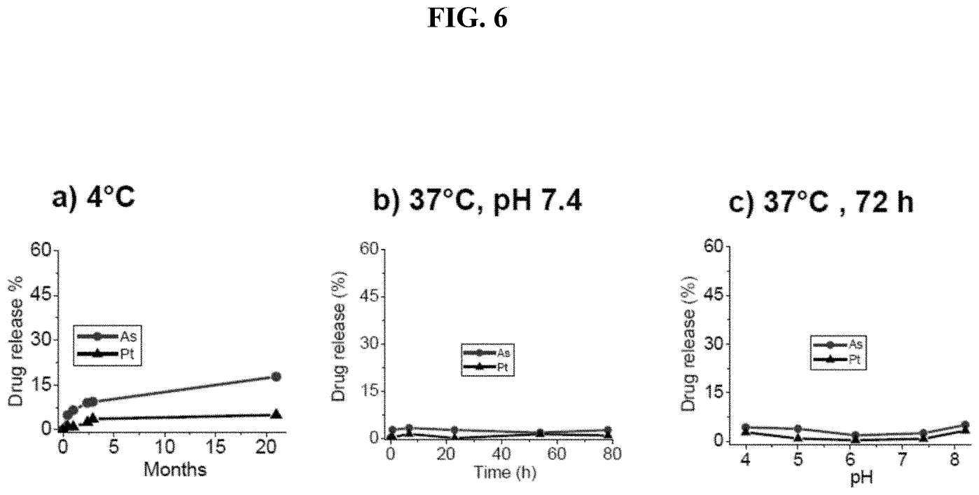

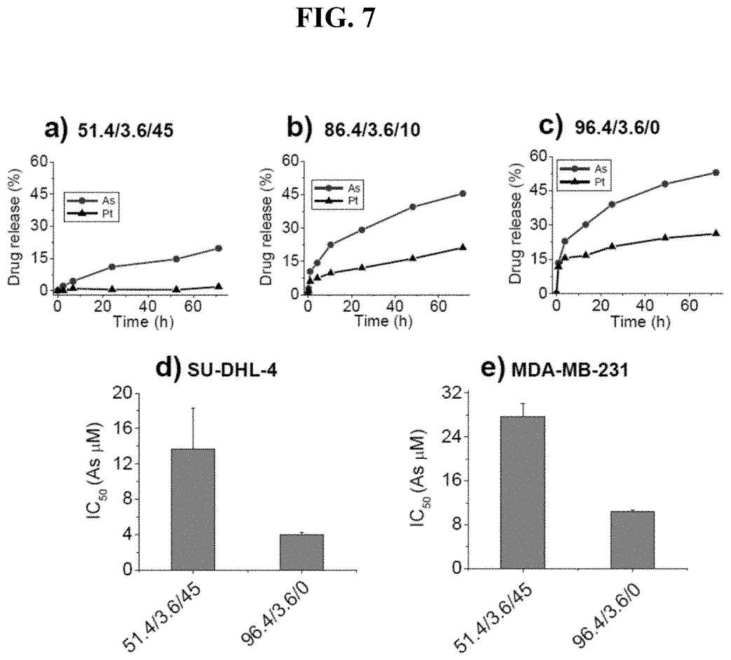

Drug release assay. NB(As, Pt) samples were kept at 4° C. or 37° C. at different pHs with 1 mM lipid. An extraliposomal buffer of 300 mM sucrose (or NaNO₃) and 10 mM MES was used for maintaining the pH at 5.0 and 6.1; for pH 7.4 and 8.2, 20 mM Bicine was additionally added; and for pH 4.0, 20 mM acetic acid was additionally added. The pHs of the dispersions were re-adjusted to the indicated values with HNO₃ or NaOH solution. For serum samples, NB(As, Pt) were mixed with FBS in a volume/volume ratio of 2:8 (80% serum) with 1 mM lipid and kept at 37° C. At various time points, aliquots were passed over a Sephadex G-50 column to remove arsenic and platinum species which had leaked from liposomes. The excluded volume fractions containing liposomes were digested with concentrated HNO₃ (trace metal grade, Fisher Scientific) before ICP-OES analysis for determination of the drug-to-lipid molar ratios. The drug release percentage was calculated as [(rₒ−rᵢ)/rₒ]×100%, where rₒ is the initial drug-to-lipid molar ratio and rᵢ the drug-to-lipid molar ratio at a specific time point (Chen et al. J. Am. Chem. Soc. 2006, 128, 13348-13349.) (SEE FIGS. 6 and 7).

Cell culture. SU-DHL-4 (human lymphoma B cells) was from the Deutsche Sammlung von Mikroorganismen and Zellkulturen GmbH (DSMZ, Braunschweig, Germany). MM.1S (human multiple myeloma B cells) was previously established in the laboratory of Prof. Steven. T. Rosen (Robert H. Lurie Comprehensive Cancer Center, Northwestern University). IM-9 (human lymphoblast B cells), MDA-MB-231 (human breast adenocarcinoma cells), OVCAR-3 (human ovary adenocarcinoma cells), KB (human nasopharyngeal epidermal carcinoma cells, FR⁺), and MCF-7 (human breast carcinoma cells, FR⁻) (Sonvico et al. J. Drug Del. Sci. Tech. 2005, 15, 407-410., Chen et al. Mol. Cancer Ther. 2009, in press., herein incorporated by reference in their entireties) were purchased from ATCC (Manassas, Va., USA). All Cells were maintained at 37° C. in an incubator with 5% CO₂ and harvested in the exponential phase of growth. SU-DHL-4, IM-9, and MM.1S cells were cultured in RPMI-1640 supplemented with 10% FBS, 2 mM glutamine, 100 units/mL penicillin-streptomycin, and 2.5 μg/mL Amphotericin B. MDA-MB-231 cells were cultured in DMEM/F12 supplemented with 5% cds-FBS, 2 mM glutamine, 100 units/mL penicillin-streptomycin, and 0.1% human insulin solution. OVCAR-3 cells were cultured in RPMI-1640 supplemented with 20% FBS, 100 units/mL penicillin-streptomycin, and 0.1% human insulin solution. MCF-7 cells was cultured in EMEM supplemented with 10% FBS, 2 mM glutamine, 100 units/mL penicillin-streptomycin and 2.5 μg/mL Amphotericin B. KB cells were cultured in EMEM supplemented with 10% FBS and 50 units/mL penicillin-streptomycin. For folate-targeting experiments, KB cells were transferred into folate-deficient RPMI-1640 medium for more than one week before each experiment (Lee et al. J. Biol. Chem. 1994, 269, 3198-3204., herein incorporated by reference in its entirety).

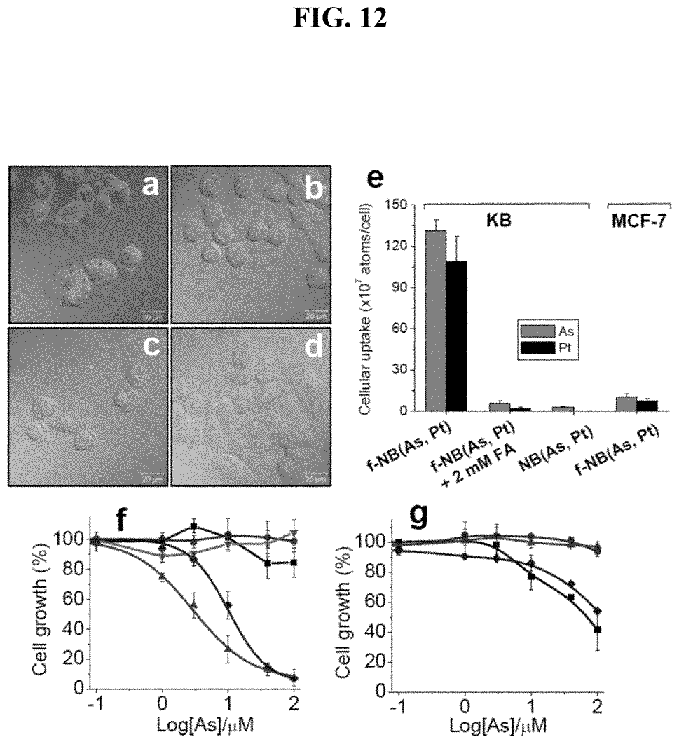

Confocal microscopy for visualization of cellular uptake. Cells were first plated, 24-48 h before each experiment, on 22-mm coverslips inside 6-well plates. Cells were then exposed to rhodamine (Rh)-labeled liposomes at 37° C. for 3 h at a lipid concentration of 40 μM and an arsenic concentration of 24 μM. After drug-containing medium removal, cells were washed with PBS×4 and fixed with PBS-buffered 4% paraformaldehyde at 20° C. for 5 min, then washed with PBS×1. Next, the coverslips were mounted on slides coated with PBS. Microscopic visualization of cells was performed using a Zeiss confocal laser scaning microscope (Carl Zeiss LSM 510, Jena, Germany). For rhodamine (Rh), maximum excitation was obtained from the 543-nm line of a He-Ne laser, and fluorescence emission intensities >570 nm were observed using a long-pass barrier filter LP-570. A water immersion objective, C-Apochromat 63×1.2 W corr. (Zeiss), was used. Cells were also imaged by light microscopy using differential interference contrast (DIC). The data are shown in FIG. 12A-D.

### Example 2

### Coencapsulation of Arsenic and Platinum Drugs within Liposomes

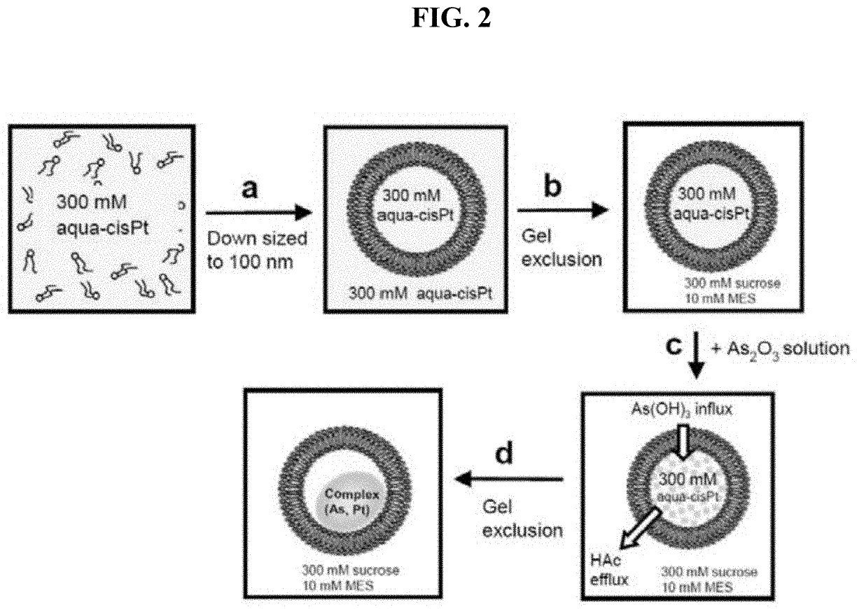

Preparation of aqua-cisPt gradients. The dried lipid film (DPPC/DOPG/Chol=51.4/3.6/45 mol %) was hydrated in 300 mM aqua-cisPt acetate (SEE FIG. 2A) to form multilamellar vesicles, which were further subjected to 7 freeze-and-thaw cycles (freezing in a ethanol/dry-ice bath and thawing in a water bath at 50° C.) (MacDonald et al. Biochim Biophys Acta 1994, 1191, 362-370., herein incorporated b reference in its entirety). The liposomes were then extruded with a manual mini-extruder (Avanti Lipids, Ala., USA), 10 times through two stacked polycarbonate filters of 0.1 μm pore size at ca. 40° C. Extruded liposomes in the aqua-cisPt acetate were then fractionated on Sephadex G-50 columns (1 mL sample volumes were placed on columns with at least a 20 mL column bed) equilibrated with the buffer of 300 mM sucrose (or NaNO₃), 10 mM MES, pH 5.1 (SEE FIG. 2B).

Arsenic loading. Typically, for 30 mg of DPPC/DOPG/Chol, 51.4/3.6/45 mol %, after removal of extraliposomal platinum species using the Sephadex G-50 column (FIG. 2b), 180 μL of arsenic trioxide solution (As 300 mM) was added to these aqua-cisPt acetate-containing liposomes at an initial As/lipid molar ratio of ˜4 and a lipid concentration of ˜5 mM, and the pH of mixture was adjusted to 6.6-6.9 (FIG. 2c). Samples were incubated at 50° C. with frequent vortexing. At various time points, 100 μL aliquots were passed through Sephadex G-50 with the same buffer at pH 7.4-8.0 to remove unencapsulated arsenic and platinum species. The concentrations of lipids (P) and of encapsulated As and Pt in the excluded fractions were determined with an inductively coupled plasma optical emission spectrometer (ICP-OES) (Vista MPX, USA) (Chen et al. J. Am. Chem. Soc. 2006, 128, 13348-13349., herein incorporated by reference in its entirety). The molar ratios of As/lipid, Pt/lipid and As/Pt were calculated and used to assess extent of loading at each time point.

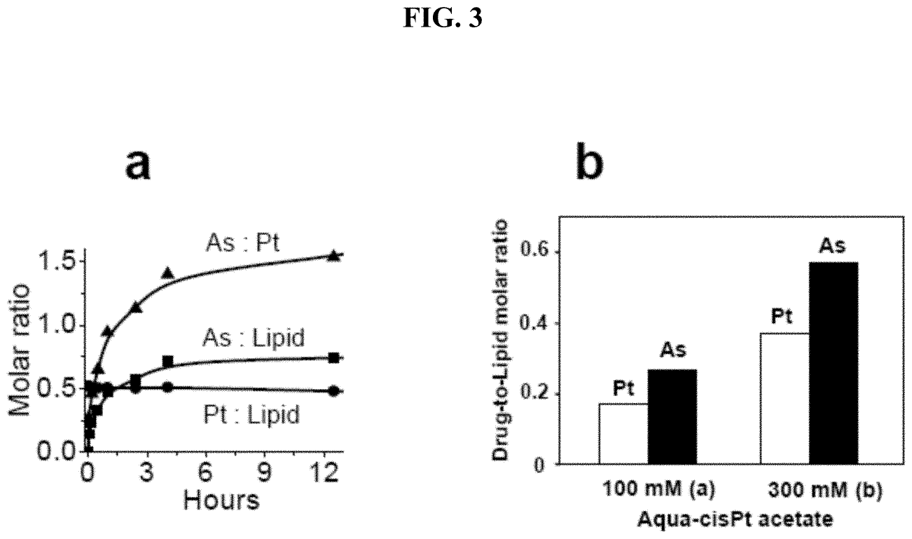

The dependence of As/lipid, Pt/lipid, and As/Pt molar ratios as a function of incubation time during arsenic loading into liposomes at 50° C. in response to the transmembrane gradient of aqua-cisPt acetate was monitiored (SEE FIG. 3A). The As/lipid molar ratio rapidly increased within the first 3 h indicating rapid arsenic loading, and achieved equilibrium after 11 h at molar ratios of As/lipid=0.66 and As/Pt=1.4 and a half-time of 40 min.

The extent of arsenic loading into liposomes increased with increasing concentration of intraliposomal aqua-cisPt acetate (SEE FIG. 3B). When 100 mM aqua-cisPt acetate was used as the intraliposomal media, a value for As/lipid (mol)=0.27 was obtained after 11 h, compared with the As/lipid (mol)=0.66 in the case of 300 mM aqua-cisPt acetate.

Intraliposomal drug concentration. The kinetics of arsenic loading into 100-nm-liposomes using 300 mM aqua-cisPt acetate as the intraliposomal medium revealed little reduction in the Pt/lipid molar ratio during the loading period of 11 h at 50° C. (SEE FIG. 3A), indicating that the platinum species are efficiently retained inside the liposomes and that little leakage occurred (<10%). The molar ratio of Pt/lipid 0.48±0.06 at 11 h can be assumed to correspond to 300 mM platinum species within one single liposome of NB(As, Pt). The molar ratio of As/Pt=1.3±0.1 indicates intraliposomal As=390±30 mM. These values are close to those expected for an encapsulated volume of 1.5 L/mol phospholipid (Haran et al. Barenholz, Biochim Biophys Acta 1993, 1151, 201-215.). The mean sizes of NB(As, Pt) were determined as 112±9 nm by dynamic light scattering on a Zetasizer Nano ZS (Malvern Instruments, Malvern, UK). The average intraliposomal core of NB(As, Pt) can be assumed as a sphere with a diameter of ˜100 nm and a volume of 5.23×10⁻¹⁹ L, considering that the thickness of a lipid bilayer is 4-6 nm (Lewis & Engelman, J. Mol. Biol. 1983, 166, 211-217., Burger Koert et al. Nat Med 2002, 8, 81-84., herein incorporated by reference in its entirety). Thus, there are ˜12×10⁴ As atoms and ˜9×10⁴ Pt atoms per liposome in NB(As, Pt).

### Example 3

### Preparation of Sterically Stabilized Arsenic and Platinum Liposomes

100 mg of dried lipid film of DSPC/DSPE-PEG₂₀₀₀/DPPE-Rh/Chol 50.5/4/0.5/45 mol % was hydrated in 2.2 mL of 300 mM aqua-cisPt acetate at 55-60° C. for 1 h. This was subjected to 6 freeze-and-thaw cycles and then extruded 10 times through two stacked polycarbonate filters of 0.1 μM pore size at 40° C. After removal of extraliposomal platinum with Sephadex G-50 using a buffer of 150 mM NaNO₃, 10 mM MES, pH 5.1, 340 μL of 300 mM As₂O₃ solution was added to these platinum-encapsulated liposomes (−6 mL), and the pH of mixture was adjusted to 7.2. This was incubated at 55° C. for 10 h with gently stirring. The mixture was then cooled down and passed through Sephadex G-50 with 300 mM sucrose, 20 mM HEPEs, pH 7.4 to remove unencapsulated arsenic and platinum species. The concentrations of lipids (P) and of encapsulated As and Pt in the excluded fractions (˜6.5 mL) were determined with an inductively coupled plasma optical emission spectrometer (ICP-OES) (Vista MPX, USA) (Chen et al. J. Am. Chem. Soc. 2006, 128, 13348-13349, herein incorporated by reference in its entirety). The molar ratios of As/lipid (0.54), Pt/lipid (0.39) and As/Pt (1.38) were calculated and used to assess extent of loading for these PEGlated liposomes NB(As, Pt). The loading extent indicates the PEGlation on liposomes didn't impair the efficiency of drug loading.

### Example 4

### Preparation of Folate-Targeted Arsenic and Platinum Liposomes

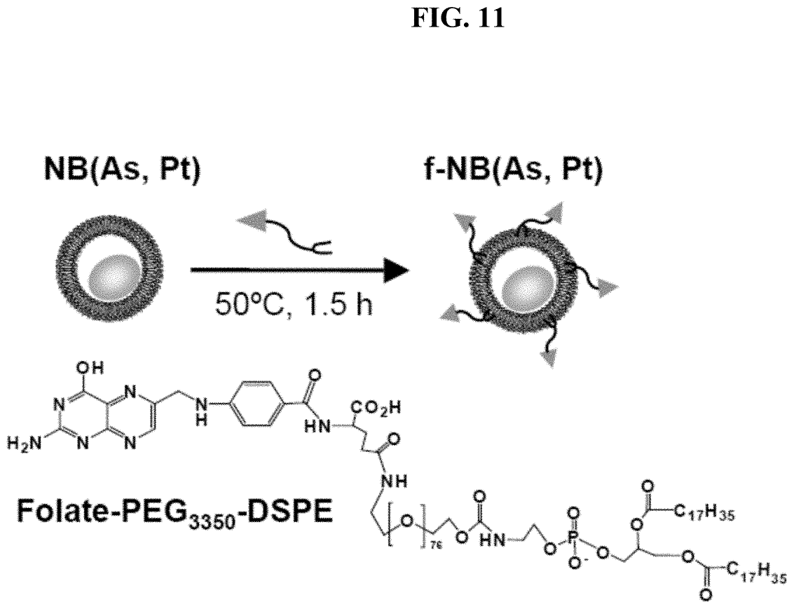

The folate-targeting ligand, DSPE-PEG₃₃₅₀-Folate, was incorporated into the lipid bilayer of pre-formed NB(As, Pt) (DPPC/DOPG/Chol=51.4/3.6/45 mol %) using the “post-insertion” technique (FIG. 11) (Allen et al. Cell. Mol. Biol. Lett. 2002, 7, 889-894., herein incorporated by reference in its entirety). Typically, 20 μL of DSPE-PEG₃₃₅₀-Folate (5 mg/mL chloroform solution) was placed in a glass tube and the chloroform was removed by rotary evaporation. The resultant lipid film was subsequently placed under a high vacuum overnight to remove any residual solvent. To this dry lipid film was added a suspension of pre-formed NB(As, Pt) (mean size 112±9 nm) at a molar ratio of folate/lipid=0.7%, in 300 mM sucrose, 10 mM MES, pH 6.4. The mixture was kept at 50° C. for 1.5 h with stirring and then passed through a Sepharose CL-4B column to remove any leaked drugs and unincorporated DSPE-PEG₃₃₅₀-Folate. The obtained folate-targeted liposomes (f-NB(As, Pt)) were analyzed by ICP-OES for arsenic, platinum and lipid concentrations, yielding molar ratios of 0.59±0.01 for As/lipid and 0.48±0.02 for Pt/lipid. The mean liposome diameters were determined as 129±4 nm by dynamic light scattering on a Zetasizer Nano ZS (Malvern Instruments, Malvern, UK), 17 nm larger than those of NB(As, Pt). The folate content of f-NB(As, Pt) preparations was determined by lysing the liposomes with 5% SDS and measuring the UV absorbance at 285 nm (Saul et al. Controlled Release 2003, 92, 49-67., herein incorporated by reference in its entirety). All samples in 5% SDS were prepared in a 96-well half-area microplate (Greiner Bio-one GmbH, Germany), 150 μL per well. To ensure every well had the same background, standard solutions (0-0.4 μM free folic acid (FA)) were additionally mixed with NB(As, Pt) at the same lipid level as in f-NB(As, Pt). The plate was then kept in the dark for 24-48 h and centrifuged at 500 g, 5-10 min (to eliminate bubbles) before reading the absorbance at 285 nm. The FA concentration of f-NB(As, Pt) was derived from the standard curve, giving the molar ratio of 0.56 (±0.15)% folate/lipid.

### Example 5

### Cytotoxicity Assay

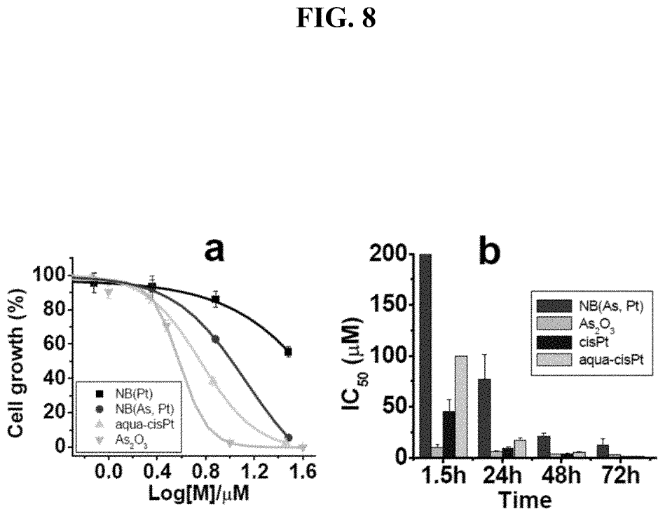

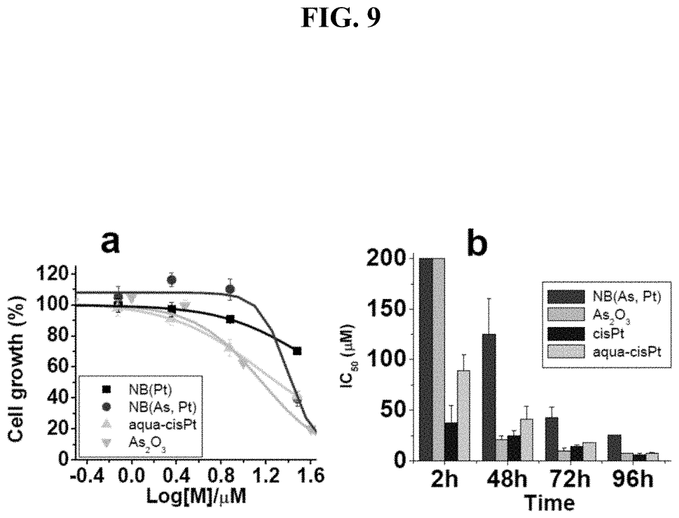

The in vitro cytotoxicities of NB(As, Pt), NB(Pt), As₂O₃, aqua-cisPt acetate and cisPt were determined using the MTS cell proliferation assay as described previously (Sekhon et al. Assay Drug Dev. Technol. 2008, 6, 711-721., herein incorporated by reference in its entirety). The MTS assay is one of most high throughput and economical assays for detecting viability of cancer cells. Briefly, SU-DHL-4 and IM-9 (40,000 cells/mL), and MM.1S (250,000 cells/mL) were treated with drugs and plated in quadruplicate (100 uL/well) onto 96-well plates. For MDA-MB-231 and OVCAR-3, 20,000 cells/mL were plated in quadruplicate (100 uL/well) onto 96-well plates, incubated overnight and then treated with drugs. After incubation for the indicated period at 37° C., the MTS/PMS solution (20 μL/well) was added to each well and the plates were further incubated for 4 h at 37° C. before reading the absorbance at 490 nm. Cell growth rates were expressed as a function of drug concentration on a logarithmic scale. The IC₅₀ values (the drug concentration required for 50% inhibition of cell growth) were determined by fitting to a sigmoidal dose-response curve using Origin 6.0 software (Microcal Software, Inc., Northampton, USA). In the case of the 1.5 h (SEE FIG. 8B) or 2 h (SEE FIG. 9B) time periods, the cells treated with drugs were first incubated for 1.5 h or 2 h at 37° C., then washed twice with PBS to remove un-associated drugs or liposomes, and further incubated up to 48 h (FIG. 8b) or 72 h (FIG. 9b) in drug-free medium, followed by the MTS assay. The data are shown in FIGS. 8-10 and Table 1.

SU-DHL-4 cells, after a 48-h incubation, NB(As, Pt) (IC_{50 20.6} μM As or 15.6□ μM Pt) exhibited attenuated cytotoxicity relative to the free drugs As₂O₃ (3.7 μM As), aqua-cisPt (5.5 μM Pt), and cisPt (3.5 μM Pt) (SEE FIG. 8A). Notably, NB(As, Pt) was three times more cytotoxic than NB(Pt) (42.4 μM Pt), indicating the bioavailability of both arsenic and platinum species from NB(As, Pt). At long incubation times (>48 hrs, i.e.), the cytotoxicities of NB(Pt, As) increased greatly, approaching those of As₂O₃, aqua-cisPt and cisPt (SEE FIG. 8B), consistent with gradual release of the drugs at 37° C. in serum (SEE FIG. 7A).

MDA-MB-231 cells, after a 72 h incubation, NB(As, Pt) (IC₅₀ 35.0 μM As or 26.6 μM Pt) exhibited attenuated cytotoxicity relative to the free drugs As₂O₃ (10.0 μM As), aqua-cisPt (17.9 μM Pt) and cisPt (14.0 μM Pt) (SEE FIG. 9A). Notably, NB(As, Pt) was 7 times more cytotoxic than NB(Pt) (>200 μM Pt), indicating the bioavailability of both arsenic and platinum species from NB(As, Pt). At long incubation times (>72 hrs, i.e.), the cytotoxicities of NB(As, Pt) increased greatly, approaching those of As₂O₃, aqua-cisPt and cisPt (SEE FIG. 9B), consistent with gradual release of the drugs at 37° C. in serum (SEE FIG. 7A).

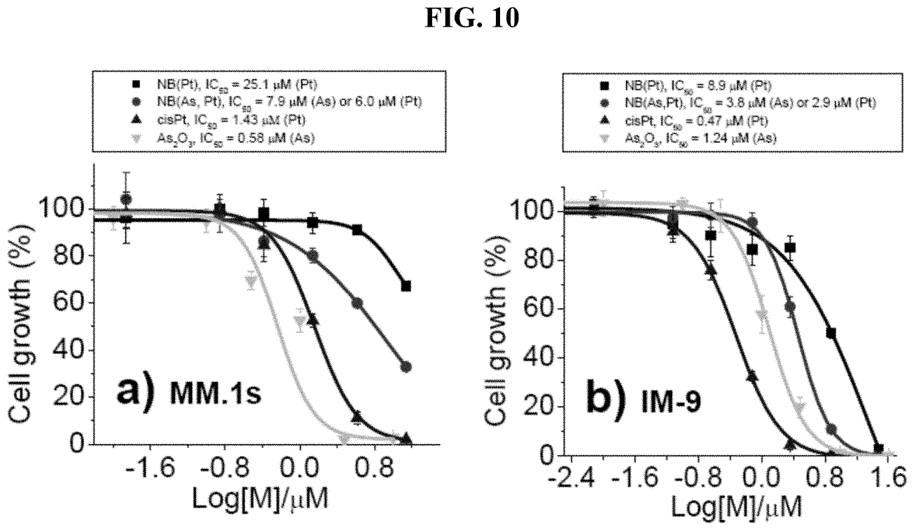

Similar anticancer activities of NB(As, Pt) were observed for other lymphoma (IM-9), multiple myeloma (MM.1S), and ovary cancer (OVCAR-3) cells (SEE FIG. 10 and Table 2), with NB(As, Pt) being 3-7 times more cytotoxic than NB(Pt).

TABLE 2
Comparison of cytotoxicities (IC₅₀) of aqua-cisPt and cisPt towards human tumor cells

| | IC₅₀ (μM)^{[b]} | | | | | |
|---|---|---|---|---|---|---|
| | | | NB(Pt)^{[c]} | Aqua-cisPt | CisPt | As₂O₃ |
| | NB(As, Pt)^{[c]} | | (Pt) | (Pt) | (Pt) | (As) |
| SU-DHL-4 | 20.6 ± 3.6 | (As) | | | | 3.7 ± 0.2 |
| | 15.6 ± 2.8 | (Pt) | 42.4 ± 15.0 | 5.5 ± 0.4 | 3.5 ± 0.8 | |
| IM-9 | 5.0 ± 0.6 | (As) | | | | 2.1 ± 0.03 |
| | 3.8 ± 0.4 | (Pt) | 10.7 ± 1.9 | 1.0 ± 0.01 | 0.6 ± 0.2 | |
| MM.1S | 7.9 | (As) | | | | 0.58 |
| | 6.0 | (Pt) | 25.1 | — | 1.43 | |
| MDA-MB-231 | 35.0 ± 7.4 | (As) | | | | 10.0 ± 2.4 |
| | 26.6 ± 5.6 | (Pt) | >200 | 17.9 ± 0.1 | 14.0 ± 1.8 | |
| OVCAR-3 | 8.8 ± 1.6 | (As) | | | | 2.7 ± 1.1 |
| | 6.6 ± 1.2 | (Pt) | 18.4 ± 0.6 | 2.6 ± 0.8 | 2.0 ± 0.01 | |

^{[a]}48 h-drug treatment for SU-DHL-4 and 1M-9, and 72 h-drug treatment for MM.1S, MDA-MB-231 and OVCAR-3 cells.
^{[b]}IC₅₀ (±SD) values are based on 2-5 independent experiments.

### Example 6

### Folate-Mediated Anticancer Efficacy

The in vitro cytotoxicities of folate-targeted liposomes against KB and MCF-7 cells were determined by Guave ViaCount assay (Stearns et al. Cancer Res. 2006, 66, 673-681., Donaldson et al. Cell Death Differ 2009, 16, 125-138., herein incorporated by reference in their entireties) (The MTS assay is not applicable in this case because the folate moiety interferes with the reactions of MTS/PMS agents and seriously impairs accuracy of cell viability (Chen et al. Mol. Cancer Ther. 2009, in press.)). Cells were first plated in 48-well plates at a density of 30,000-60,000 cells/mL, 0.2 mL per well. After 24 h at 37° C., the media were replaced with drug solutions. Cells were thus incubated with drugs continuously for 72 h, or exposed to drugs for 3 h at 37° C., then washed with PBS×2 and further incubated up to 72 h in drug-free medium. The same Pt concentrations of aqua-cisPt were used as in f-NB(As, Pt). Cell viability was determined by Guave ViaCount (Stearns et al. Cancer Res. 2006, 66, 673-681., Donaldson et al. Cell Death Differ 2009, 16, 125-138., herein incorporated by reference in their entireties), using a Guava EasyCyte Mini flow cytometer (Guava Technologies, Hayward, Calif.). Cells, after released from plates by trypsinization (using 0.05% trypsin/0.02% EDTA), were stained with the Guava Viacount agent, which contains two fluorescent dyes, one cell permeable and one impermeable. This allows viable and dead cell numbers to be accurately determined by Guava Viacount software. Cell growth rates were expressed as a function of drug concentration on a logarithmic scale. The ICso values (the drug concentration required for 50% inhibition of cell growth) were determined by fitting to a sigmoidal dose-response curve using Origin 6.0 (Microcal Software, Inc., Northampton, USA) (SEE FIGS. 12F-G and Table 3).

TABLE 3
Cytotoxicity (IC₅₀) of folate-targeted arsenic and platinum liposomes to human tumor cells^{[a]}

| | KB (FR⁺), IC₅₀ (μM) | | | | MCF-7 (FR⁻), IC₅₀ (μM) | | |
|---|---|---|---|---|---|---|---|
| Formulations | 3 h | | 72 h | | 3 h | 72 h | |
| As₂O₃ | >200 | | 5.2 ± 1.0 | | 63.7 ± 22.3 | 6.9 ± 1.7 | |
| NB(As, Pt) | >200 | | 20.1 ± 0.03 | (As) | >200 | 40.8 ± 1.3 | (As) |
| | | | 15.8 ± 0.02 | (Pt) | | 32.1 ± 1.1 | (Pt) |
| f-NB(As, Pt) | 3.3 ± 1.4 | (As) | 1.2 ± 0.01 | (As) | >200 | 34.8 ± 4.5 | (As) |
| | 2.6 ± 1.1 | (Pt) | 1.0 ± 0.01 | (Pt) | | 27.4 ± 3.5 | (Pt) |
| f-NB(As, Pt) + | >200 | | 13.1 ± 1.0 | (As) | — | — | |
| 2 mM FA | | | 10.3 ± 0.7 | (Pt) | | | |
| Aqua-cisPt | 9.7 ± 2.5 | | 1.3 ± 0.2 | | 88.9 ± 14.4 | 12.2 ± 0.4 | |

^{[a]}Cells were incubated with drugs continuously for 72 h, or exposed to drugs for 3 h, then washed and further incubated up to 72 h at 37° C. in drug-free medium before IC₅₀ measurement. IC₅₀ values (±SD) are based on As or Pt concentration (μM), from 2-3 independent experiments.

### Example 7

### Quantitative Analysis of Drug Uptake

Cells were plated, 24 h before each experiment, in 6-well plates with 500,000 cells per well. Cells were then exposed to 10 μM arsenic as free drug or within liposomes for 3 h at 37° C. The same Pt concentration (8 μM) of aqua-cisPt were used as in f-NB(As, Pt). After washing with PBS to remove non-associated drugs, cells were released from tissue culture plates with 0.05% trypsin/0.02% EDTA (Invitrogen), followed by 3×PBS washing (centrifugation, 500 g, 5 min, 4° C.). Two control samples were taken for cell number determination with the Guava ViaCount Assay, using a Guava EasyCyte Mini flow cytometer (Guava Technologies, Hayward, Calif.). Cell pellets from each well were digested with 100 μL concentrated nitric acid (trace metal grade, Fisher Scientific) for measurement of arsenic (As) and platinum (Pt) concentrations by inductively coupled plasma mass spectroscopy (ICP-MS, X Series II, Thermo Electron, UK). Cell-associated drug was expressed as As and Pt atoms per cell (SEE FIG.12E and Table 4.

TABLE 4
Comparison of cellular uptake of various drug formulations to tumor cells

| | Cellular uptake (x10⁷ atoms/cell)^{[a]} | | | |
|---|---|---|---|---|
| | KB (FR⁺) | | MCF-7 (FR⁻) | |
| Formulations | As | Pt | As | Pt |
| As₂O₃ | 17.1 ± 1.0 | | 9.4 ± 2.3 | |
| NB(As, Pt) | 3.2 ± 0.4 | 0.26 ± 0.06 | 1.7 ± 0.3 | 0.18 ± 0.06 |
| f-NB(As, Pt) | 131.3 ± 8.1 | 108.9 ± 18.3 | 10.2 ± 2.3 | 7.3 ± 1.7 |
| f-NB(As, Pt) + | 5.8 ± 1.7 | 1.8 ± 1.4 | 2.8 ± 1.5 | 0.7 ± 0.7 |
| 2 mM FA | | | | |
| Aqua-Pt | | 0.4 ± 0.1 | | 0.24 ± 0.07 |

^{[a]}Cellular uptake was measured by ICP-MS after cells were exposed to drugs for 3 h at 37° C. The mean values and standard deviations (±SD) are based on three independent experiments.

### Example 8

### Materials and Methods

### Materials

Lipids of Dipalmitoylphosphatidylcholine (DPPC), Dioleoylphosphatidylcholine (DOPC), Dioleoylphosphatidylglycerol (DOPG), Dipalmitoylphosphatidylethanolamine-poly(ethylene-glycol)(2000) (DPPE-PEG2000), Distearoylphosphatidylethanolamine-PEG2000-Maleimide (DSPE-PEG2000-Mal) were purchased from Avanti Polar Lipids (Alabaster, Ala., USA). Cholesterol (Chol), nickel(II) acetate, cobalt(II) acetate, copper(II) acetate, zinc(II) acetate, HEPES and NaCl were obtained from Sigma (Milwaukee, Wisc., USA). Nickel(II) sulfate was from Mallinckrodt (Kentucky, USA). Sephadex G50 and Sepharose CL-4B were from Sigma. The chimeric murine/human anti-CD20 Rituxan was a generous gift from Dr. Steven Rosen (Robert H. Lurie Comprehensive Cancer Center, Northwestern University).

### Methods

### Preparation of Liposomes

All liposomes were made from either DPPC/DOPG/Chol (65:5:30, wt %) or DOPC/DOPG/Chol (65:5:30, wt %) and were prepared by extrusion methods (unless stated otherwise). Lipids, at the indicated ratios, were dissolved in chloroform. The chloroform was removed from the mixtures by gentle vacuum evaporation and subsequently, the lipids films were placed under a high vacuum for at least 4 h to remove any residual solvent. The dried samples were hydrated in the indicated solution to form multilamellar vesicles (MLVs), which were further subjected to ten freeze-and-thaw cycles (freezing in ethanol/dry-ice bath, −70° C. and thawing in water bath, 50° C.). The resulting MLVs was extruded 10 times through stacked polycarbonate filters of 0.4 and 0.1 or 0.8 um pore size at 50-60° C. using a manual mini-extruder (Avanti Lipids, Ala., USA). This gave a mean liposome size between 80 and 180 nm as determined by light scattering.

### Preparation of Ion Gradients

The downsized liposomes prepared in the indicated solution were fractionated on Sephadex G-50 columns (1 mL sample volumes were placed on columns with at least a 20 mL column bed) equilibrated with various buffers. The buffers used for the external liposome included 150 mM or 300 mM NaCl and 20 mM HEPES, 300 mM sucrose and 20 mM HEPES at the indicated pH.

### Methods for Quantification of Drug Loading

A concentrated solution of sodium arsenite (400 mM, pH 7.4) or arsenic trioxide (150 mM, pH 12.5) was added to the liposome dispersion (typically, 5 mM lipids) after the creation of an ion gradient. At various time points, aliquots were removed and passed through a Sephadex G-50 column to separate the unencapsulated drug from the encapsulated drug. The concentrations of lipids (P), encapsulated As and M (Ni, Co, Cu, or Zn) in the excluded fractions were determined by an Inductively Coupled Plasma Optical Emission Spectrometer (ICP-OES). The molar ratios of As/Lipid, M/lipid and As/M were calculated and used to assess loading efficiency.

### Determination of Intraliposomal Concentration

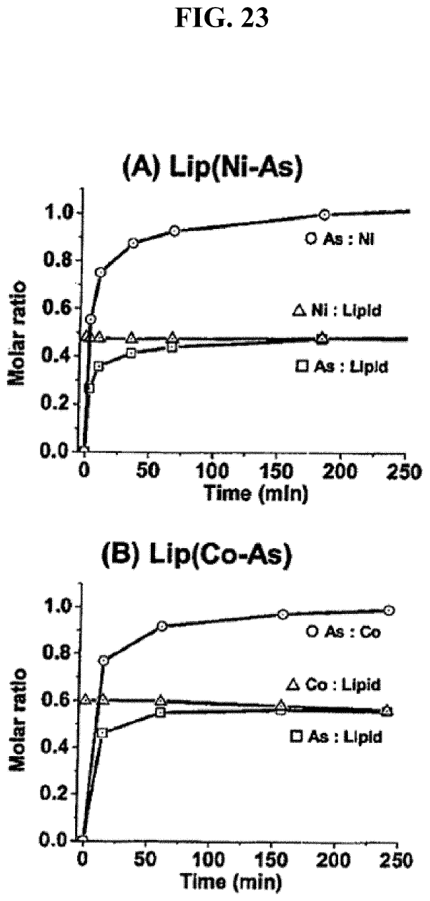

Based on the kinetics of arsenic loading into 100-nm-liposomes using nickel(II) acetate (FIG. 23A), cobalt(II) acetate (FIG. 23B), copper(II) acetate, or zinc(II) acetate as intraliposomal medium, the metal ions (M²⁺) inside liposomes are greatly retained with little leakage (<10%) within the loading period of 5 h at 50° C. The M-to-Lipid molar ratio of Lip(M—As) products can be assumed to correspond to the initial metal ion concentration. This, combined with the As-to-lipid molar ratio, is used to calculate the arsenic concentration inside the liposome. Typically, in the preparation of Lip(Ni—As) drug using 300 mM Ni(O₂CCH₃)₂ as intraliposomal medium, the Ni-to-Lipid molar ratio was found to be 0.5 at 2.5 h, which corresponds to the 300 mM Ni²⁺ within one single liposome, and the As-to-Lipid molar ratio was 0.5, indicating there is 300 mM As³⁺ within the same vesicle. A similar method was used to calculate the intraliposomal concentration of As³⁺ and M²⁺ for other Lip(M—As) drugs where 300 mM cobalt(II) acetate, copper(II) acetate or zinc(II) acetate was used as intraliposomal medium. The results are compared in FIG. 18.

### Drug Release Assay

The in vitro arsenic release assay was carried out with liposome lipid concentrations of 0.9-2.6 mM. Samples were kept at 4° C. or 37° C. at the indicated pH. The extraliposomal buffer of 300 mM NaCl or 300 mM sucrose and 20 mM HEPES was used for pH 7.0-7.4; for pH 5.0-5.5, 30 mM MES was additionally added; and for pH 4.0, 40 mM acetic acid was additionally added. At the indicated time-points, aliquots were placed into a Sephadex G-50 column to remove arsenic drug, which leaked out from liposomes. The drug-to-lipid molar ratio in the excluded liposome fractions was determined as above. The drug release percentage (%) was calculated as [(rₒ−rᵢ/rₒ]×100%, rₒ, initial As-to-Lipid molar ratio, rᵢ, the remained As-to-Lipid molar ratio at a specific time point.

### Transmission Electron Microscopy

Liposome dispersions were imaged by transmission electron microscopy (TEM) at low dose. For negative stained TEM, the liposome samples were stained using 4% uranyl acetate and air-dried for 3 h before TEM loading. The TEM column vacumn is 1.0×10⁻⁶ Pa.

### Thiolation of Rituxan

Rituxan was washed 4 times using a buffer of 150 mM NaCl, 20 mM HEPES, pH 7.1 (degassed under N₂) in Microcon-10. The concentration was determined by Bio-Rad Protein Assay. Purified Rituxan (10 mg/mL) was thus incubated with 2-iminothiolane in O₂-free buffer of 150 mM NaCl, 20 mM HEPES, pH 8.0 at a ratio of 20:1 mol/mol for 1 h at room temperature. This was followed by washing 4 times using 150 mM NaCl, 20 mM HEPES, pH 7.1 (degassed under N₂) in Amicon Ultra-4. The concentration of thiolated Rituxan was determined by Bio-Rad Protein Assay.

### Determination of Rituxan/Liposome Ratio: CBQCA Assay

The amount of Rituxan coupled to the liposomes was determined by using a CBQCA (3-(4-carboxybenzoyl)quinoline-2-carboxalde-hyde) assay, where an increase in fluorescence is observed when CBQCA agent binds to a free amino group on the protein (You, W. W. et al. Anal. Biochem. (1997) 244: 277-282). Briefly, 5 mg of CBQCA was dissolved in 410 □L of Dimethylsulfoxide (DMSO). Aliquots (5-15 uL) of Rituxan-liposomal-arsenic were mixed with 10 uL of CBQCA solution and 5 uL of 20 mM KCN in the presence of 100 mM sodium borate buffer at pH 9.3 with a final volume of 150 uL. The reactions were carried out in a 96-well microplate. After 2 h incubation at room temperature with gentle shaking and protected from light, the relative fluorescence was determined on a Synergy HT Multi-detection Microplate Reader (EM 528 nm, EX 485 nm). Antibody concentration was determined from a standard curve of the known concentrations of free Rituxan with the presence of similar amount of lipids.

### Cell Culture Experiments

The human lymphoma B cell line of SU-DHL-4 (CD20-positive) was obtained from the American Type Culture Collection (Rockville, Md., USA). Cells were cultured in RPMI 1640 (Invitrogen Corporation) supplemented with 10% fetal bovine serum, 2 mM glutamine, 100 units/mL penicillim, 100 ug/mL streptomycin, and 2.5 ug/mL fungizone. Cells were maintained at 37° C. in an incubator with 5% CO₂ and harvested in the exponential phase of growth.

### Example 9

### Arsenic Acids (H3AsO3) Pass Across Liposomal Membrane too Rapidly for Drug Delivery Application

The following experiment demonstrates why standard liposome loading methods will not work for arsenic drugs.

30 mg of dried lipid film of DPPC/DOPG/Chol (65/5/30, wt %) was hydrated in 0.9 mL of 150 mM sodium arsenite, or 75 mM As2O3 at pH 7.5 (pH was adjusted by concentrated HCl and 5 M NaOH) for 1.5 hat 50° C. For these two solutions, the major arsenic species is H₃AsO₃, (FIG. 13). This was subjected to 10 freeze-and-thaw cycles and then extruded 10 times through stacked polycarbonate filters of 0.4 and 0.1 um pore size at 50° C. After removal of the extra liposomal arsenic species with Sephadex G-50 using a buffer of 150 mM NaCl, 20 mM HEPES, pH 7.0, the dispersion of sodium arsenite or As₂O₃-encapsulated liposomes (2.0 mL) was kept on a 4° C. ice bath. At various time points, 125-200 uL aliquots were passed through Sephadex G-50 to remove arsenic species that leaked out from liposomes. At each time point, the extent of encapsulated drug was determined as As/Lipid molar ratio as described in Example 8. The arsenic release % against the time is plotted in FIG. 15, showing that the encapsulated arsenic species very readily release both in the cases of sodium arsenite and arsenic trioxide, with half-times <50 min at 4° C. and >90% leakage after 24 h. This half-life is too short for appropriate pharmacokenetics and seriously limits the shelf-life of drugs.

### Example 10

### Arsenic Loading Using Metal Ion Gradients

15-20 mg of dried lipid film of DPPC/DOPG/Chol (65/5/30, wt %) was hydrated in 0.5-0.6 mL of 300 mM Ni(O₂CCH₃)₂, Ni(NO₃)₂, NiCl₂ and NiSO₄ and 142 mM Ni(O₂CH)₂ at pH 6.8 (the pH of Ni(II) salts were adjusted by concentrated HCl or NaOH solution when necessary) for 1.5 h at 50° C., respectively. This was subjected to 10 freeze-and-thaw cycles and then extruded 10 times through stacked polycarbonate filters of 0.4 um and 0.1 um pore size at 50-60° C. After removal of extraliposomal nickel salts with Sephadex G-50 using a buffer of 300 mM (150 mM for the case of Ni(O₂CH)₂) NaCl and 20 mM HEPES, pH 6.8, 60-90 uL of 150 mM arsenic trioxide was added to these nickel-encapsulated liposomes (1.5-1.8 mL) at a lipid concentration of 5 mM, and the pH of mixture was adjusted to 7.2. This was incubated at 50° C. with frequent vortexing. At various time points, 80-130 uL aliquots were passed through Sephadex G-50 with the same buffer at pH 4.0 to remove unencapsulated arsenic and nickel species. The extent of encapsulated drug at each time point was determined as As/Lipid molar ratio as described in Example 8. FIG. 16 shows the kinetics of arsenic loading into liposomes using various salts of nickel(II) as an intraliposomal medium.

For the cases of 300 mM Ni(O₂CCH₃)₂ and 142 mM Ni(O₂CH)₂, efficient arsenic loading with As-to-lipid molar ratios of 0.5 and 0.22, respectively, was achieved after 60 min with a half time of <5 min. For 300 mM NiSO₄, there was little uptake of arsenic even after one week. For 300 mM NiCl₂, the arsenic uptake was very slow with the half time of 650 min and achieved a final As-to-lipid ratio of 0.35 after 24 h; for 300 mM Ni(NO₃)₂, the uptake appeared to be three times faster than that of NiCl₂ with a half-time of 228 min, and achieved a final As-to-lipid molar ratio of 0.5 after 10 h.

### Example 11

### Arsenic Loading Using Copper Ion Gradients

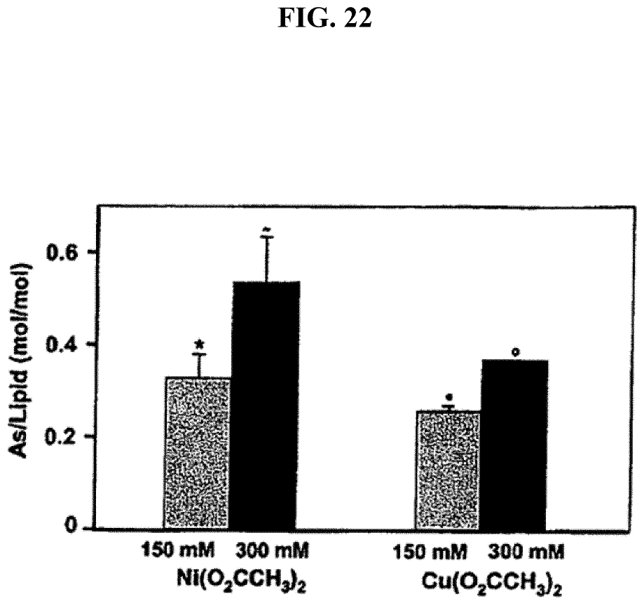

30 mg of dried lipid film of DPPC/DOPG/Chol (65/5/30, wt %) was hydrated in 0.9 mL of 150 mM Cu(O₂CCH₃)₂, pH 5.4 for 1.5 h at 50° C. This was subjected to 10 freeze-and-thaw cycles and then extruded 10 times through stacked polycarbonate filters of 0.4 um and 0.1 um pore size at 60° C. After removal of extraliposomal Cu(O₂CCH₃)₂ with Sephadex G-50 using a buffer of 150 mM NaCl, 20 mM HEPES, pH 5.1, 100 uL of 150 mM arsenic trioxide was added to these copper-encapsulated liposomes (2.0 mL) at a lipid concentration of 5 mM and the mixture was adjusted to pH 6.0. This was incubated at 50° C. with frequent vortexing. At various time points, 200 uL aliquots were passed through Sephadex G-50 with the same buffer at pH 3.8 to remove unencapsulated arsenic and copper species. The extent of encapsulated drug at each time point was determined as As/Lipid molar ratio as described in Example 8. The loading was complete after 1 h with a half-life of 8 min. The final As/Lipid molar ratio is 0.24. A similar experiment was carried out using 300 mM Cu(O₂CCH₃)₂ as intraliposomal medium, which gave the final As/Lipid molar ratio of 0.37 (FIG. 22).

### Example 12

### Arsenic Loading Using Cobalt Ion Gradients

30 mg of dried lipid film of DPPC/DOPG/Chol (65/5/30, wt %) was hydrated in 0.9 mL of 300 mM Co(O₂CCH₃)₂, pH 7.2 for 1.5 h at 50° C. This was subjected to 10 freeze-and-thaw cycles and then extruded 10 times through stacked polycarbonate filters of 0.4 um and 0.1 um pore size at 60° C. After removal of the extraliposomal Co(O₂CCH₃)₂ with Sephadex G-50 using a buffer of 300 mM NaCl, 20 mM HEPES, pH 6.9, 100 uL of 150 mM arsenic trioxide was added to these cobalt-encapsulated liposomes (1.8 mL) and the mixture was adjusted to pH 7.3. This was incubated at 50° C. with frequent vortexing. At various time points, 200 uL aliquots were passed through Sephadex G-50 with the same buffer at pH 5.4 to remove encapsulated arsenic and cobalt species. The extent of encapsulated drug at each time point was determined as As/Lipid molar ratio as described in Example 8. FIG. 23(B) shows the kinetics of arsenic loading into liposomes using 300 mM Co(O₂CCH₃)₂ as intraliposomal medium. The loading was completed after 1 h with the half-life of 8 min and the final As/Lipid molar ratio is 0.6.

### Example 13

### Arsenic Loading Using Zinc Ion Gradients

60 mg of dried lipid film of DPPC/DOPG/Chol (65/5/30, wt %) was hydrated in 1.6 mL of 300 mM zinc acetate, pH 6.1, for 1.5 h at 50° C. This was subjected to 10 freeze-and-thaw cycles and then extruded 10 times through stacked polycarbonate filters of 0.1 um pore size at 60° C. After removal of the extraliposomal zinc acetate with Sephadex G-50 using a buffer of 300 mM sucrose, 20 mM HEPES, pH 5.9, 107 uL of 400 mM NaAsO₂ was added to these zinc-encapsulated liposomes (2.9 mL) and the mixture was adjusted to pH 6.4. This was incubated at 50° C. with frequent vortexing. 0.2 mL of aliquot at a time of 1 h, and 0.5 mL at 2 h and 4 h were withdrawn and passed through Sephadex G-50 with the same buffer at pH 4.0 to remove unencapsulated arsenic and zinc species. The loading equilibrium was achieved after 1 h. The extent of encapsulated drug was determined as As/Lipid molar ratios of 0.23. A similar experiment was carried out with the addition of an amount of arsenic trioxide into the zinc acetate encapsulated liposomes, which gave the final As/Lipid molar ratios of 0.21.

### Example 14

### Stability of Liposome Components

The sample of As—Ni-encapsulated liposomes with a 0.5 As-to-lipid molar ratio and 2.6 mM lipids in the outer buffer of 300 mM sucrose, 20 mM HEPES, pH 7.2 was kept at 4° C. At various time points, 150 uL aliquots were passed through Sephadex G-50 with the same buffer at pH 4.0 to remove the extraliposomal arsenic. The drug release % at each time point was determined as described in Example 8. FIG. 20A shows there is little leakage of arsenic (<5%) after six months of storage at 4° C. at pH 7.2.

The sample of As—Co-encapsulated liposomes with a 0.5 Co-to-lipid molar ratio and 0.9 mM lipids in the outer buffer of 300 mM NaCl, 20 mM HEPES, pH 7.2 was kept at 4° C. At various time points, 330 uL aliquots were passed through Sephadex G-50 with the same buffer at pH 4.0 to remove the extraliposomal arsenic. The drug release % at each time point was determined as described in Example 8. FIG. 20B shows there was little leakage of arsenic (<5%) after six months of storage at 4° C. at pH 7.2.

### Example 15

### Arsenic Release Triggered by temperature and Intracellular pH Gradients

The samples of As—Ni-encapsulated- or Co—As-encapsulated-liposomes with a 0.6 As-to-lipid ratio and 1.0-1.7 mM lipids were kept at 37° C. in a buffer of 300 mM NaCl, 20 mM HEPES at pH 7.2, pH 5.0 (+30 mM MES) or pH 4.0 (+40 mM acetic acid). At various time points, 200-390 □L aliquots were passed through Sephadex G-50 with the same buffer at pH 4.0 to remove the extraliposomal arsenic. The drug release % at each time point was determined as described in Example 8 (FIG. 20).

FIG. 20A shows that for Lip(Ni-As) liposomes at pH 7.2, there was 15% release after 24 h incubation at 37° C., compared with little release at 4° C. When the pH was decreased to 5.0, 55% arsenic was released after 24 h at 37° C.; when the pH was further decreased to 4.0, the release was 24× faster than that at pH 5.0, with 50% release after 1 h and over 95% release after 13 h. FIG. 20B shows that for Lip(Co—As) liposomes at pH 7.2, there was 14% release after 24 h incubation at 37° C., compared with little release at 4° C.; when pH was decreased to 5.2, 50% arsenic was released after 24 h at 37° C.; when pH was further decreased to 4.0, the release was 40× faster than that at pH 5.2, with 50% release after 0.6 h and over 90% release after 1.1 h.

### Example 16

### Arsenic Loading into Liposomes with Fluid Lipids

60 mg of dried lipid film of DOPC/DOPG/Chol (65/5/30, wt %) was hydrated in 1.6 mL of 300 mM Ni(O₂CCH₃)₂, pH 6.9 for 1 h at 37° C. This was subjected to 10 freeze-and-thaw cycles and then extruded 10 times through stacked polycarbonate filters of 0.4 um and 0.08 um pore size at 40° C. After removal of the extraliposomal Ni(O₂CCH₃)2 with Sephadex G-50 using a buffer of 300 mM NaCl, 20 mM HEPES, pH 6.9, 210 uL of 150 mM arsenic trioxide was added to these nickel-encapsulated liposomes (5 mL) and the mixture was adjusted to pH 7.2. This was incubated at 37° C. with frequent vortexing. A 0.4 mL of aliquot at a time of 1 h, 3.8 mL at 2.5 h and 0.5 mL at 4 h were withdrawn and passed through Sephadex G-50 with the same buffer at pH 4.0 to remove unencapsulated arsenic and nickel species. The extent of encapsulated drug at each time point was determined as As/Lipid molar ratio as described in Example 8 with an As-to-Lipid ratio of 0.86 (1 h), 1.0 (2.5 h), and 1.0 (4 h). The results indicate the loading was almost complete after 1 h at 37° C.

Stability and release assay. The samples of As—Ni-encapsulated liposomes of DOPC/DOPG/Chol (65/5/30, wt %) with a 1.0 As-to-lipid molar ratio and 1.0 mM lipids in the outer buffer of 300 mM NaCl, 20 mM HEPES, pH 7.2 were kept in at 4° C. and 37° C. At various time points, 300-330 uL aliquots were passed through Sephadex G-50 with the same buffer at pH 4.0 to remove the extraliposomal arsenic. The drug release % at each time point was determined and plotted against time (FIG. 21). There is 16% release of arsenic from liposome after 100 h of storage at 4° C. and pH 7.2. The release was ten times faster when stored at 37° C. where 16% arsenic was released after only 9 h.

Preparation of immunoliposomal arsenic PEGlated and Maleimided Liposome(Ni—As). 35.4 mg of a dried lipid film of DPPC/DPPE-PEG2000/DSPE-PEG2000-Mal/Chol (66.4/2.6/1/30, mol%) was hydrated in 1.1 mL of 300 mM Ni(O₂CCH₃)₂, pH 6.9 for 1.5 h at 50° C. This was subjected to 10 freeze-and-thaw cycles and then extruded 10 times through stacked polycarbonate filters of 0.4 um and 0.08 um pore size at 60° C. After removal of extraliposomal Ni(O₂CCH₃)₂ with Sephadex G-50 using a buffer of 300 mM NaCl, 20 mM HEPES, pH 6.9, 130 uL of 150 mM arsenic trioxide was added to the liposome dispersion (2.4 mL) and the pH of mixture was adjusted to 7.3. This was incubated at 50° C. for 2.5 h with frequent vortexing. The mixture was adjusted to pH 4.0, and allowed to passed through Sephadex G-50 with the same buffer at pH 4.0 to remove unencapsulated arsenic and nickel species. The pH values of the excluded fractions were adjusted back to 7.2. The extent of encapsulated drug was determined as the As-to-Lipid molar ratio of 0.33, as described in Example 8.

### Rituxan-Liposome(Ni—As).

To the PEGlated and Maleimided Liposomal(Ni—As) complex (2.7 mL), thiolated Rituxan (freshly prepared, see Example 8) was added at a molar ratio of 1:223 (Rituxan/Lipid). This was stirred overnight at room temperature in an O₂-free environment. The mixture was then passed through Sepharose CL-4B using a buffer of 300 mM NaCl, 20 mM HEPES, pH 7.1 to separate the unconjugated Rituxan. The density of Rituxan coupled to liposomes was determined as 30.1 ug Rituxan/umol Lipid, as described in Example 8. This is conversed to 19 Rituxan molecues per liposome, based on the assumption that there are approximately 7.7×10¹² liposomes at 100 nm scale per □mol of lipids (Hansen, C. B., et al. Biochim. Biophys. Acta (1995) 1239: 133-144). The As-to-Lipid molar ratio is 0.24.

### Example 17

The aggregation of arsenic drugs inside liposomes can be reversed under specific conditions. Thus, the active drug has the potential to be released from liposome-arsenic conjugates once they are delivered to tumor cells. The following example demonstrates that targeted liposomal arsenic drugs are as effective as the parent drug for killing tumor cells but with lower toxicity towards healthy cells, through lipid coating and antibody delivery.

### Cytotoxicity Assays

The in vitro cytotoxicity of free As₂O₃, free Rituxan, Liposome(Ni—As), and Rituxan-Liposome(Ni—As) on human lymphoma cell line SU-DHL-4 was determined using a MTS (3-(4,5-dimethylthiazol-2-yl)-5-(3-carboxymethoxyphenyl)-2-(4-sulfophenyl)-2H-tetrazolium) assay as described previously (Lopes de Menezes, D. E. et al. Biochim. Biophys. Acta (2000) 1466: 205-220). Cells (25,000 cells/mL) were treated with various drugs and plated in 96-well plates. After incubation for 72 h at 37° C., the MTS solution (20 uL/well) was added to each well and the plates were further incubated for 4 h at 37° C. before the absorbance readings at 490 nm. The IC₅₀ values (the drug concentration required for 50% inhibition of cell growth) were determined based on simple fit curves of cell growth % against drug concentration (arsenic level): As₂O₃, 1.45 uM; Liposome(Ni—As), 1.92 uM; Rituxan-Liposome(Ni—As), 1.52 uM. This indicates that after a three-day exposure at 37° C., the encapsulated arsenic had similar cell killing effects as that of free As₂O₃, through releasing from Liposome(Ni-As), or from Rituxan-Liposome(Ni—As). A similar amount of free Rituxan to that of Rituxan-Liposome(Ni—As) was used to treat cells for comparison. It was found that there was no significant influence of free Rituxan (<3000 ng/mL) on cell growth.

In a parallel experiment, SU-DHL-4 cells (100,000 cells/mL) were treated with various drugs and plated in 96-well plates. After incubation at 37° C. for 20 min, cells were washed twice using 200 uL/well of PBS and refilled with 100 uL/well of fresh medium and incubated for an additional 71.6 h. The MTS solution (20 uL/well) was added to each well and the plates were further incubated for 4 h at 37° C. before taking the absorbance readings at 490 nm. The inhibited growth of cells in the presence of various drugs are displayed in FIG. 24, showing there was no significant effect from Liposome(Ni—As) and free Rituxan when compared with that of the free As₂O₃ and the Rituxan-Liposome(Ni—As). The Rituxan conjugation improved the inhibition effect of Liposome(Ni—As). This indicates that within the first 20 min exposure to free As₂O₃ at 37° C., the cells might already accumulate a significant amount of arsenic since the H₃AsO₃ has high permeability through the membrane. When sheltered by the liposome bilayer, the possibility of arsenic reaching cells is greatly reduced, indicating that lipid coating could prevent the killing of healthy cells. Through conjugating to the mAb of Rituxan, liposome(Ni—As) could be delivered to cells by Rituxan binding to the CD20 antigen on the cell surface. This was followed by the release of arsenic from liposomes, leading to inhibition of cell growth.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A composition comprising a liposomal nanoparticle, wherein said liposomal nanoparticle encapsulates therapeutically effective amounts of a platinum-containing compound and an arsenic-containing compound.

2. The composition of claim 1, wherein said arsenic-containing compound is selected from arsenic trioxide, arsenite, arsenious acid, arsonous acid, arsine, thioarsenious acid, arsenate, arsenic acid, arsenic acid, arsenic acid, methylarsinic acid, and dimthylarsinic acid.

3. The composition of claim 1, wherein said platinum-containing compound is selected from Cisplatin (cisPt), Monoaqua-cisPt, Aqua-cisPt, Carboplatin, Oxaliplatin, and platinum coordinating compounds.

4. The composition of claim 1, wherein said liposomal nanoparticle is stable under physiological conditions.

5. The composition of claim 1, wherein said liposomal nanopartcle further comprises a targeting moiety.

6. The composition of claim 5, wherein said targeting moiety comprises a targeting ligand.

7. The composition of claim 6, wherein said targeting ligand is selected from folic acid, retinoic acid, a peptide, an estrogen analog, transferrin, and granulocyte-macrophage colony stimulating factor.

8. The composition of claim 5, wherein said targeting moiety comprises an antibody.

9. The composition of claim 8, wherein said antibody is selected from Rituxan, HERCEPTIN, CAMPATH-1H, HM1.24, anti-HER2, Anti-CD38, HuM195, HP67.6, TRAIL mAb, transferin, anti-uPA, and prolactin.

10. A composition comprising particles having the molecular formula:
[(X₁X₂X₃X₄)Pt]ₙ[(Y₁Y₂Y₃Y₄)As]ₘ
or
[(X₁X₂X₃X₄)Pt]ₙ[(Y₁Y₂Y₃)As]ₘ
wherein X=O, OH, OH₂, N, NH₂, NH₃, S, SH, Cl, Br, F, P, Se, SeH, an amino carrier ligand, a leaving group, or an R group; wherein Y=O, OH, OH₂, N, NH₂, NH₃, S, SH, Cl, Br, F, P, Se, SeH, As, an amino carrier ligand, a leaving group, or an R group; wherein R comprises an alkyl group or an alkylidene group; wherein n is 10 or less; wherein m is 10 or less; wherein X is optionally bound to additional substituents; and wherein Y is optionally bound to additional substituents.

11. The composition of claim 10, wherein said particles comprise liposome-encapsulated nanoparticles.

12. The composition of claim 11, wherein said liposome-encapsulated nanoparticles are stable under physiological conditions.

13. The composition of claim 11, wherein said liposome-encapsulated nanoparticles further comprise a targeting moiety.

14. The composition of claim 13, wherein said targeting moiety comprises a targeting ligand.

15. The composition of claim 14, wherein said targeting ligand is selected from folic acid, retinoic acid, a peptide, an estrogen analog, transferrin, and granulocyte-macrophage colony stimulating factor.

16. The composition of claim 13, wherein said targeting moiety comprises an antibody.

17. The composition of claim 16, wherein said antibody is selected from Rituxan, Herceptin, Campath-1H, HM1.24, HER2, Anti-CD38, HuM195, HP67.6, TRAIL mAb, transferin, anti-uPA, and prolactin.

18. A method for making a pharmaceutical preparation comprising:
a) providing:
i) a lipid composition;
ii) a platinum-containing compound; and
iii) an arsenic-containing compound;
b) combining said lipid composition and said platinum-containing compound under conditions such that said lipid compositions forms liposomes, wherein said liposomes encapsulate said platinum-containing compound; and
c) combining said liposomes and said arsenic-containing compound under conditions such that said arsenic-containing compound is co-encapsulated with said platinum containing compound within said liposomes.

19. The method of claim 18, further comprising the step between steps (b) and (c) of purifying said liposomes away from unencapsulated platinum-containing compound.

20. The method of claim 18, further comprising the step following step (c) of purifying said liposomes away from unencapsulated arsenic-containing compound.
